(19) United States
(12) Patent Application Publication
(10) Pub. No.: US 2018/0344805 A1
(43) Pub. Date: Dec. 6, 2018

(54) GLYCOLIPID COMPOUNDS AND THEIR USES IN THE TREATMENT OF TUMOURS

(71) Applicants: Agalimmune Limited (GB/GB), London (GB); Kode Biotech Limited (NZ/NZ), Auckland (NZ)

(72) Inventors: Graham GRIFFITHS, London (GB); Stephen SHAW, London (GB); Nicolai Vladimirovich BOVIN, Moscow (RU); Alexander Borisovich TUZIKOV, Moscow (RU); Elena Yurievna KORCHAGINA, Moscow (RU); Stephen HENRY, Auckland Ellerslie (NZ)

(21) Appl. No.: 15/771,692

(22) PCT Filed: Nov. 15, 2015
(86) PCT No.: PCT/RU2015/000766

§ 371 Date: Apr. 27, 2018

(51) Int. Cl. A61K 38/14; C07K 9/00; C07K 14/00; C07H 15/04; A61K 31/7028; A61K 38/16; A61K 45/06; A61P 35/00

(52) U.S. Cl. CPC A61K 38/14; C07K 9/00; C07K 14/001; A61P 35/00; A61K 31/7028; A61K 38/16; A61K 45/06; C07H 15/04

(57) ABSTRACT

The invention relates to novel glycolipid compounds and pharmaceutical compositions comprising said glycolipids and to processes for preparing said glycolipids. The invention also relates to said glycolipids for use in treating tumours and methods of treating tumours using said glycolipids.

### FIELD OF THE INVENTION

The invention relates to novel glycolipid compounds and pharmaceutical compositions comprising said glycolipids and to processes for preparing said glycolipids. The invention also relates to said glycolipids for use in treating tumours and methods of treating tumours using said glycolipids.

### BACKGROUND OF THE INVENTION

The major cause of death in cancer patients with solid tumours is the recurrence of the cancer after surgery as multiple metastases are non-resectable and/or refractory to any therapy. The majority of these patients are considered to have a terminal cancer disease. As no treatment is available for them, many of these patients die within weeks or a few months after detection of metastatic tumour lesions.

Tumours develop in cancer patients because the immune system fails to detect tumour cells as cells that ought to be destroyed. Tumour cells express autologous tumour antigens in a large proportion of cancer patients. These autologous tumour antigens may elicit a protective anti-tumour immune response. Tumour cells, or tumour cell membranes, have to be internalized by antigen presenting cells in order to induce the development of an anti-tumour immune response. However, the immune system in cancer patients displays “ignorance” toward the tumour antigens that is associated with early development of the tumour in a “stealthy” way, so it is “invisible” to antigen presenting cells (Pardoll D M. Clin. Immunol. 2000; 95:S44-49; and Dunn G P et al. Nat Immunol 2002; 3: 991-8).

In addition, the tumour microenvironment and local cytokine milieu are often suppressive toward immune function and can actively induce immune cell anergy and death (Malmberg K J. Cancer Immunol. Immunother. 2004; 53: 879-92; Lugade A A et al. J. Immunol. 2005; 174: 7516-23). Effective treatment of such metastatic tumour lesions requires two components:
1. Destruction of the lesions that are large enough to be detected visually or by imaging technology, and
2. Induction of a protective anti-tumour immune response against tumour antigens.

Such an immune response results in immune-mediated detection, regression, and/or destruction of micrometastases which cannot be detected visually and are not detectable by imaging.

Induction of a protective anti-tumour immune response requires uptake of the tumour cells or cell membranes by antigen presenting cells and their transportation to the draining lymph nodes, where the antigen presenting cells process the tumour antigen molecules. The majority of these tumour antigens are specific to the individual patient. The immunogenic tumour antigen peptides are presented by antigen presenting cells in association with class I or class II MHC molecules for the activation of tumour specific CDS⁺ and CD4⁺ T cells, respectively. Only after these T cells are activated by the processed and presented tumour antigen peptides, can these lymphocytes proliferate, leave the lymph nodes, circulate in the body, seek and destroy metastatic tumour cells expressing tumour antigens. In addition, though only after they are activated, helper T cells can provide help to B cells for producing antibodies against the tumour antigens. However, since the tumour cells naturally evolve to be “invisible” to antigen presenting cells, the developing tumour metastases are usually ignored by the immune system to the extent that metastasizing tumour cells can proliferate even within lymph nodes. Therefore, eliciting an effective anti-tumour immune response requires effective targeting of tumour cells to antigen presenting cells.

What is needed are compositions and methods to introduce compounds into a tumour, such as by non-surgical or surgical methods, under conditions such that the compound will insert into tumour cell membranes and a naturally occurring antibody will interact with the introduced compound. It is believed that such interaction will induce local inflammation for the regression and/or destruction of the tumour and the targeting of the tumour cells and/or tumour cell membranes to antigen presenting cells. This process will elicit a protective immune response in the host against tumour cells expressing the tumour antigens in micrometastases that cannot be detected visually or by imaging and therefore cannot be removed by resection.

US 2006/251661 describes methods of administering natural glycolipid compounds to tumour lesions that induce local expression of α-Gal epitopes within the tumour which interact with the natural anti-Gal antibody.

There is therefore a need to provide alternative glycolipid compounds which are capable of being delivered directly into a tumour in order to activate an immune response against the tumour.

### SUMMARY OF THE INVENTION

According to a first aspect of the invention, there is provided a glycolipid compound selected from a compound of formula (I), (II) and (III) or a pharmaceutically acceptable salt thereof:

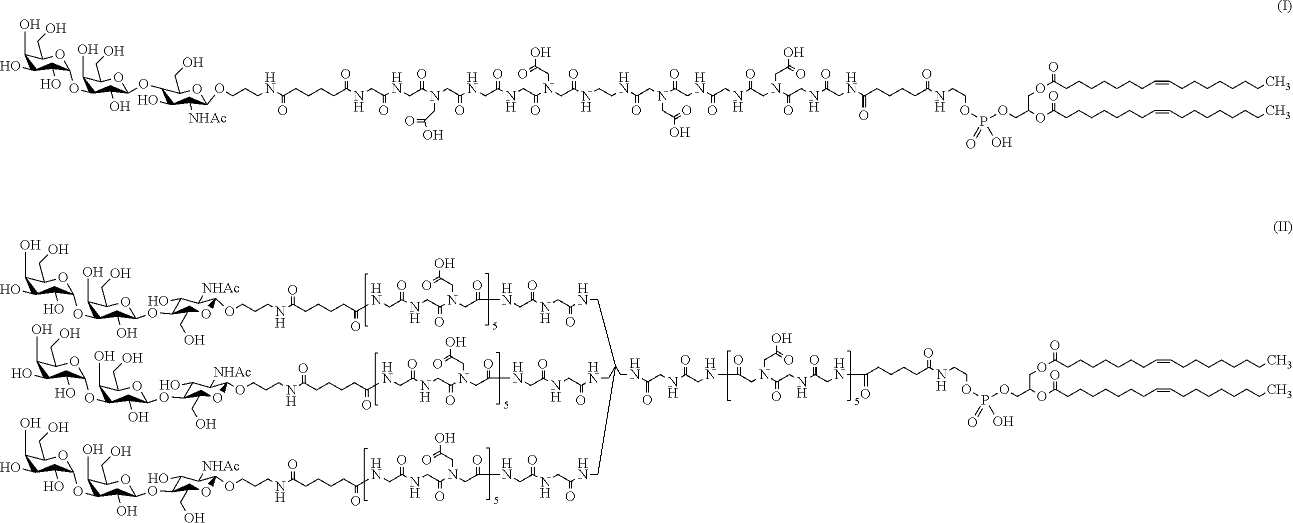

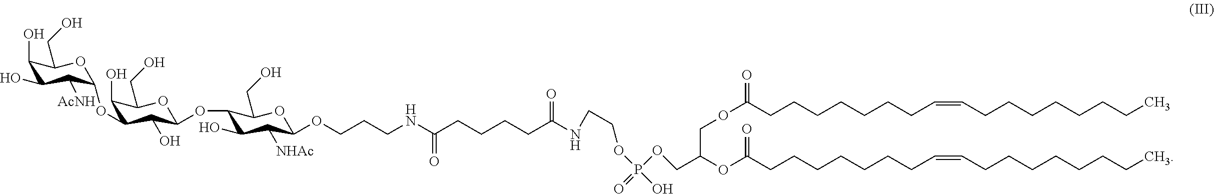

According to a further aspect of the invention, there is provided a pharmaceutical composition comprising a glycolipid compound selected from a compound of formula (I), (II) and (Ill) or a pharmaceutically acceptable salt thereof as defined herein.

According to a further aspect of the invention, there is provided a glycolipid compound selected from a compound of formula (I), (II) and (Ill) or a pharmaceutically acceptable salt thereof as defined herein or a pharmaceutical composition as defined herein for use in the treatment of a tumour.

According to a further aspect of the invention there is provided a pharmaceutical composition comprising a glycolipid compound selected from a compound of formula (I), (II) and (Ill) or a pharmaceutically acceptable salt thereof as defined herein in combination with one or more additional therapeutic agents.

According to a further aspect of the invention, there is provided a method of treating a tumour in a subject, comprising:
a) providing:
   i) a subject comprising at least one tumour that comprises a plurality of cancer cells having a cell surface; and
   ii) the glycolipid compound selected from a compound of formula (I), (II) and (Ill) or a pharmaceutically acceptable salt thereof or the pharmaceutical composition as defined herein; and
b) introducing said glycolipid or composition into the tumour.

### BRIEF DESCRIPTION OF THE FIGURES

FIG. 1: Data obtained from the Anti-Gal Recruitment Assay for the compound of formula (I) as prepared herein in Example 1 (Galili-CMG2-DOPE).

FIG. 2: Data obtained from the Anti-Gal Recruitment Assay for the compound of formula (II) as prepared herein in Example 2 (Galili-T17 DOPE).

FIG. 3: Data obtained from the Complement Dependent Cytotoxicity Assay for the compound of formula (I) as prepared herein in Example 1 (Galili-CMG2-DOPE).

FIG. 4: Data obtained from the Complement Dependent Cytotoxicity Assay for the compound of formula (II) as prepared herein in Example 2 (Galili-T17 DOPE).

FIG. 5: Data obtained from the Complement Dependent Cytotoxicity Assay for the compound of formula (III) as prepared herein in Example 3 (GalNAc-Gal-GlcNAc-Ad-DOPE).

### DETAILED DESCRIPTION OF THE INVENTION

According to a first aspect of the invention, there is provided a glycolipid compound selected from a compound of formula (I), (II) and (III) or a pharmaceutically acceptable salt thereof as defined hereinbefore.

The invention described herein provides glycolipids (i.e. the compounds of formula (I), (II) and (III)) which are capable of being inserted into the cell membrane of tumour cells within a treated tumour. It is believed that the presence of the glycolipids of the invention in the tumour lesion results in the destruction or regression of the tumour by the immune mediated inflammatory process that is induced by the interaction between the natural anti-Gal antibodies present in the subject and the α-Gal epitope of the compounds of formula (I) and (II) (prepared as described herein as Examples 1 and 2, respectively). Moreover, this treatment converts the treated tumour into a vaccine that elicits a systemic protective anti-tumour immune response that prevents the development of distant metastases by immune destruction of metastatic tumour cells.

In addition to antibodies to α-Gal, human serum also contains antibodies to other carbohydrates. Blood group A type 2 linear trisaccharide (GalNAcα1-3-Gal-β1-4GlcNAc, the GalNAc epitope) is one such glycan that can be recognised by natural antibodies in human serum (von Gunten, S. et al. (2009) J. Allergy Clin. Immunol. 123, 1268-76.e15; and Bovin (2013) Biochemistry (Moscow) 78(7), 786-797). These antibodies may also have utility in inducing immune killing of tumour cells labelled with glycolipids containing the GalNAc epitope. The glycolipid compound of formula (III) (prepared as described herein as Example 3) is a glycolipid containing the GalNAc epitope that was synthesised to assess whether antibodies present in human serum could selectively recognise cells labelled with this glycolipid and stimulate complement mediated lysis of the labelled cells.

The invention described herein comprises a therapy treatment modality that includes, but is not limited to, intratumoural delivery of a specific glycolipid, referred to the compounds of formula (I), (II) and III), that carries the α-Gal or GalNAc epitope and therefore may be referred to as an “α-Gal glycolipid” or “GalNAc glycolipid”. The α-Gal or GalNAc glycolipid inserts into the outer leaflet of the cell membrane of tumour cells within the treated lesion. The presence of α-Gal or GalNAc glycolipids in the tumour lesion achieves two goals:
1. Immune mediated destruction of tumour lesions by the inflammatory process that is induced within the tumour lesion by the interaction between the natural anti-Gal or anti-GalNAc antibody and the α-Gal or GalNAc epitopes of α-Gal or GalNAc glycolipids inserted in tumour cell membranes; and
2. Effective uptake by antigen presenting cells of tumour cells and tumour cell membranes with inserted α-Gal or GalNAc glycolipids and thus, expressing α-Gal or GalNAc epitopes that bind in situ anti-Gal or anti-GalNAc antibodies, thereby converting the treated tumour lesion into an autologous tumour vaccine.

Although it is not necessary to understand the mechanism of an invention, it is believed that this uptake results in an effective immune response against tumour antigens present on or within the tumour cells expressing α-Gal or GalNAc epitopes. It is further believed that this immune response may result in immune mediated destruction of metastatic tumour cells that do not express α-Gal or GalNAc epitopes, but express the tumour antigen.

The invention contemplates administering by injection, or any other means, compounds into tumours that induce expression of α-Gal or GalNAc epitope on cells within the treated tumour. Such administration of α-Gal or GalNAc glycolipids achieves the following objectives:
1. The binding of the natural anti-Gal or anti-GalNAc antibody to α-Gal or GalNAc epitopes of α-Gal or GalNAc glycolipids may result in local complement activation, thereby generating chemotactic factors including, but not limited to, C5a and C3a. These chemotactic factors induce an extensive migration of antigen presenting cells such as, but not limited to, dendritic cells and macrophages into the tumour tissue.
2. The lipid tails of α-Gal or GalNAc glycolipids will spontaneously insert into the tumour cell membranes within the treated lesion, resulting in expression of α-Gal or GalNAc epitopes on tumour cells. Anti-Gal or anti-GalNAc binding to these epitopes is believed to induce regression and/or destruction of tumours comprising tumour cells.
3. Opsonization of the tumour cell membranes by anti-Gal or anti-GalNAc targets them for effective uptake by antigen presenting cells that migrate into the tumour. The migration of these antigen presenting cells is directed by the chemotactic complement cleavage peptides that are generated following anti-Gal or anti-GalNAc binding to α-Gal or GalNAc glycolipids within the treated tumour.

Without being bound by any particular mechanism, it is believed that the Fc portion of the tumour cell membrane-bound anti-Gal or anti-GalNAc IgG molecules binds to Fc-gamma receptors (FcγR) on antigen presenting cells and induces uptake of the tumour cells by the antigen presenting cells. A similar induction for uptake may occur as a result of the interaction between the C3b component of complement deposits on anti-Gal or anti-GalNAc binding tumour cells and C3b receptors on antigen presenting cells. This anti-Gal or anti-GalNAc mediated targeting of tumour membranes to antigen presenting cells enables effective transport of autologous tumour antigens to draining lymph nodes, and processing and presentation of immunogenic tumour antigen peptides by antigen presenting cells within the lymph nodes.

Thus, intratumoural injection of α-Gal or GalNAc glycolipids converts a treated tumour lesion into an in situ autologous tumour vaccine that provides tumour antigens to the immune system, thereby eliciting a protective anti-tumour immune response. This immune response is capable of inducing tumour regression comprising the destruction of individual tumour cells or of small aggregates of tumour cells (i.e. for example, micrometastases). These micrometastases are usually undetectable either visually or by imaging and not accessible by conventional surgical or radiotherapy techniques (i.e. they are nonresectable because of their small size). Therefore, the present method has the added advantage that it is able to treat micrometastases which are usually undetectable either visually or by imaging and not accessible by conventional surgical and radiotherapy techniques.

### Definitions

References herein to the term “compound of formula (I)” refer to a specific example of α-Gal glycolipid which consists of a functional (F), spacer (S) and lipid (L) component and can be used to insert into cell membranes so that the cell will display the functional (F) component on its surface. The functional (F) component of the compound of formula (I) is a trisaccharide group of: Gal-α1-3-Gal-β1-4GlcNAc (i.e. the α-Gal epitope). The spacer (S) component consists of two CMG groups and the lipid (L) component is DOPE. References to a compound of formula (I) herein also include “Galili-CMG2-DOPE” and “CMG” which may be used interchangeably. The structure of the compound of formula (I) is as shown hereinbefore. The compound of formula (I) may be prepared according to the detailed synthetic procedure described herein for Example 1.

References herein to the term “compound of formula (II)” refer to a specific example of α-Gal glycolipid which consists of a functional (F), spacer (S) and lipid (L) component and can be used to insert into cell membranes so that the cell will display the functional (F) component on its surface. The functional (F) component of the compound of formula (II) is a trisaccharide group of: Gal-α1-3-Gal-β1-4GlcNAc (i.e. the α-Gal epitope). The spacer (S) component consists of a T17 group and the lipid (L) component is DOPE. References to a compound of formula (II) herein also include “Galili-T17 DOPE” and “T17” which may be used interchangeably. The structure of the compound of formula (II) is as shown hereinbefore. The compound of formula (II) may be prepared according to the detailed synthetic procedure described herein for Example 2. The trimeric compound of formula (II) is believed to contain impurities of the dimeric compound of formula (II)^{a}:

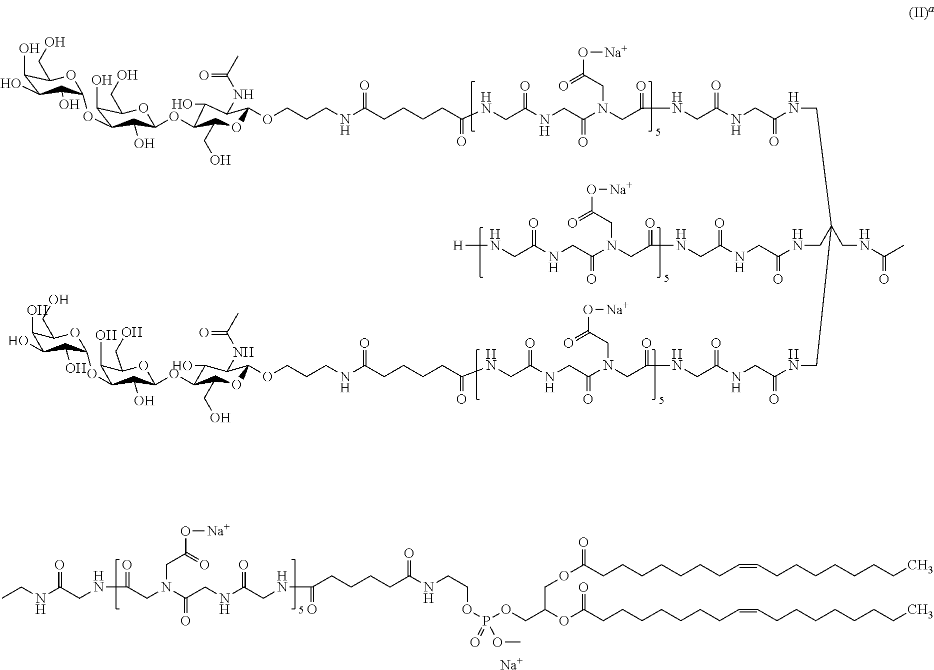

Therefore, references herein to the terms “compound of formula (II)”, “Galili-T17 DOPE” and “T17” refer to a mixture of compounds of formula (II) and (II)^{a}.

References herein to the term “compound of formula (III)” refer to a specific example of GalNAc glycolipid which consists of a functional (F), spacer (S) and lipid (L) component and can be used to insert into cell membranes so that the cell will display the functional (F) component on its surface. The functional (F) component of the compound of formula (I) is a trisaccharide group of: GalNAcα1-3-Gal-β1-4GlcNAc (i.e. the GalNAc epitope). The spacer (S) component comprises a O(CH₂)₃NH group and the lipid (L) component is DOPE. References to a compound of formula (III) herein also include “GalNAc-Gal-GlcNAc-Ad-DOPE” and “GalNAc” which may be used interchangeably. The structure of the compound of formula (III) is as shown hereinbefore. The compound of formula (III) may be prepared according to the detailed synthetic procedure described herein for Example 3.

In one embodiment, the glycolipid compound is selected from a compound of formula (I). In an alternative embodiment, the glycolipid compound is selected from a compound of formula (II). In an alternative embodiment, the glycolipid compound is selected from a compound of formula (I) and (II). In an alternative embodiment, the glycolipid compound is selected from a compound of formula (III).

References herein to the term “DOPE” refer to a phosphatidylethanolamine (PE) having the chemical name 1,2-dioleoyl-sn-glycero-3-phosphoethanolamine.

Compounds of formula (I), (II) and (III) can exist in the form of salts, for example acid addition salts or, in certain cases salts of organic and inorganic bases such as carboxylate, sulfonate and phosphate salts. All such salts are within the scope of this invention, and references to compounds of formula (I), (II) and (III) include the salt forms of the compounds.

The salts of the present invention can be synthesized from the parent compound that contains a basic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the base forms of these compounds with the appropriate base or acid in water or in an organic solvent, or in a mixture of the two; generally, nonaqueous media such as ether, ethyl acetate, ethanol, isopropanol, or acetonitrile are used.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, methanesulfonic (mesylate), ethanesulfonic, naphthalenesulfonic, valeric, acetic, propanoic, butanoic, malonic, glucuronic and lactobionic acids. One particular salt is the hydrochloride salt. Another particular salt is the hydrogensulfate salt, also known as a hemisulfate salt. In a further embodiment, the salt is selected from sodium and potassium or comprises an amine-counter-ion.

Where the compounds of formula (I), (II) and (III) contain an amine function, these may form quaternary ammonium salts, for example by reaction with an alkylating agent according to methods well known to the skilled person. Such quaternary ammonium compounds are within the scope of formula (I).

The compounds of the invention may contain a single or multiple counter-ions depending upon the pKa of the acid from which the salt is formed. For example, Example 1 contains 4 acid groups and Example 2 contains 20 acid groups, therefore, each of these compounds is well suited to containing multiple counter-ions.

The salt forms of the compounds of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

The term “α-Gal epitope”, as used herein, refers to any molecule, or part of a molecule, with a terminal structure comprising Galα1-3Galβ1-4GlcNAc-R, Galα1-3Galβ1-3GlcNAc-R, or any carbohydrate chain with a terminal Galα1-3Gal at the non-reducing end. The α-Galactosyl (also referred to as “alpha-Gal” or “α-Gal”) epitope, i.e., galactosyl-alpha-1,3-Galactosyl-beta-1,4-N-acetylglucosamine is described in Galili, U. and Avila, J. L., Alpha-Gal and Anti-Gal, Subcellular Biochemistry, Vol. 32, 1999. Xenotransplantation studies have determined that humans mount an immune response to the α-Galactosyl epitope, which itself is not normally found in humans, but is found in other animals and many microorganisms.

The term “GalNAc epitope” as used herein, refers to any molecule, or part of a molecule, with a terminal structure comprising GalNAcα1-3-Gal-β1-4GlcNAc or any carbohydrate chain with a terminal GalNAcα1-3-Gal at the non-reducing end.

The term “glycolipids”, as used herein, refers to any molecule with at least one carbohydrate chain linked to a ceramide, a fatty acid chain, or any other lipid. Alternatively, a glycolipid maybe referred to as a glycosphingolipid.

The term “anti-Gal” as used herein, refers to naturally occurring antibodies which bind the α-Gal epitope.

The term “anti-GalNAc” as used herein, refers to naturally occurring antibodies which bind the GalNAc epitope.

The term “α-1,3-Galactosyltransferase” as used herein, refers to any enzyme capable of synthesizing α-Gal epitopes.

The term “anti-Gal binding epitope”, as used herein, refers to any molecule or part of a molecule that is capable of binding, in vivo or in vitro, the natural anti-Gal antibody.

The term “anti-GalNAc binding epitope”, as used herein, refers to any molecule or part of a molecule that is capable of binding, in vivo or in vitro, the natural anti-GalNAc antibody.

The term “nonresectable”, as used herein, refers to any part of an organ or bodily structure that cannot be surgically removed. For example, a “nonresectable tumour” may be a tumour physically unreachable by conventional surgical techniques, a tumour where its removal does not improve the overall cancer disease or wellbeing of the patient, or a tumour where its removal may be detrimental to a vital organ.

The term “membrane-bound”, as used herein, refers to any molecule that is stably attached to, or embedded within, a phospholipid bilayer. Such attaching or embedding may involve forces including, but not limited to, ionic bonds, covalent bonds, hydrophobic forces, or Van der Waals forces etc. For example, a protein comprising a hydrophobic amino acid region may insert itself into a phospholipid bilayer membrane, or a molecule that contains a lipid tail can insert itself into the phospholipid bilayer of cells and become embedded. The lipid component of the α-Gal or GalNAc containing glycolipids of the invention is used to insert into the cell membranes of the tumour to create a tumour displaying the α-Gal or GalNAc epitope on its cell surface.

The term “subset”, as used herein, refers to a specialized group lower in number than the whole group. For example, a patient may present with a plurality of nonresectable solid tumours. Of this plurality, a subset may be accessible by non-surgical techniques whereas another subset may not be accessible by non-surgical techniques.

The term “accessible”, as used herein, refers to any ability to treat a solid tumour by non-surgical techniques. Such techniques may include, but are not limited to, injection into the skin or injection via endoscopy, bronchoscopy, cystoscopy, colonoscopy, laparoscopy, catheterization, or topical application by a lotion, ointment or powder. For example, an ovarian solid tumour may be accessible by laparoscopy. In another, example, a colon solid tumour may be accessible by colonoscopy.

The term “introducing”, as used herein, refers to any method of transferring a compound into a tissue and subsequently into cells within said tissue. Such methods of introduction may include, but are not limited to, viral vectors, retroviral vectors, adenoviral vectors, biobalistics, lipofection, and many commercially available DNA vectors known in the art. Alternatively, a compound may be placed adjacent to a cell such that the compound is incorporated into the cell by physiological mechanisms (i.e., for example, hydrophobic interactions or active transport). One method of introduction comprises injection, wherein a compound is placed directly into the intercellular space within the injected tissue. Such an injection may be possible when an organ part, growth (i.e., for example, a solid tumour), or bodily cavity is “accessible”.

The term “into”, as used herein, refers to the successful penetration of a molecule through or within a cell membrane. For example, a viral vector may be introduced into a solid tumour cell under conditions such that the tumour cell is transfected. In another example, a glycolipid may be introduced into a tumour cell under conditions such that the glycolipid becomes inserted into the cell's phospholipid bilayer membrane.

The terms “regression”, “is at least partially diminished in size” or “reduced”, as used herein, refer to a diminution of a bodily growth, such as, for example, a solid tumour. Such a diminution may be determined by a reduction in measured parameters such as, but not limited to, diameter, mass (i.e., weight), or volume. The diminution by no means indicates that the size is completely reduced, only that a measured parameter is quantitatively less than a previous determination.

The term “destruction”, as used herein, refers to the complete cellular breakdown of a bodily growth, such as, for example, a solid tumour. Such destruction may involve intracellular apoptosis, T cell mediated killing of cells, complement mediated cytolysis, and/or macrophage phagocytosis such that the bodily growth is completely digested and eliminated from the body. The term “destruction of a tumour” refers to the reduction of a tumour to such a degree that it is no longer detectable by diagnostic means.

The term “treating”, “treatment” and “treat” all used herein are intended to refer to a procedure which results in at least partially diminishing in size or reduction in size of a bodily growth, such as, for example, a solid tumour.

The term “fewer than all”, as used herein, refers to a subset of a group. In the context of one embodiment of the present invention, treatment of fewer than all of the tumours in a patient is contemplated. In other words, in one embodiment, it is not necessary to treat every tumour by introduction of the α-Gal or GalNAc epitope (e.g. by introduction of the α-Gal or GalNAc containing glycolipids of the invention); rather, introduction to a subset results in an immune response to all tumours (including those not directly treated). In this manner, one can achieve a collective diminution of a plurality of bodily growths, such as, for example, solid tumour metastases. Such a diminution may be determined by a reduction in measured parameters such as, but not limited to, number. The diminution by no means indicates that the parameter is reduced to zero, only that a measured parameter is quantitatively less than a previous determination.

The term “growth”, as used herein, refers to any tissue or organ that comprises a cellular mass considered to represent an abnormal proliferation. Such growths may be cancerous, non-cancerous, malignant, or non-malignant. If a growth comprises cancer, it may be a tumour.

The term “tumour” as used herein, refers to an abnormal mass of tissue which results from an abnormal growth or division of cells. Such tumours may be solid (i.e. a mass of cells in particular organ, tissue or gland, such as on the peritoneum, liver, pancreas, lung, urinary bladder, prostate, uterus, cervix, vagina, breast, skin, brain, lymph node, head and neck, stomach, intestine, colon or ovaries) or non-solid (i.e. liquid tumours which develop in the blood, such as leukaemia).

The term “subject”, as used herein, refers to any organism that is capable of developing a tumour. Such organisms include, but are not limited to, mammals, humans, non-primate mammals, prosimians and New World monkeys etc.

The term “molecule”, as used herein, refers to the smallest particle of a composition that retains all the properties of the composition and is composed of one or more atoms. These one or more atoms are arranged such that the molecule may interact (i.e., ionically, covalently, non-covalently etc.) with other molecules to form attachments and/or associations. For example, a molecule may have one or more atoms arranged to provide a capability for an interaction with an anti-Gal or anti-GalNAc antibody.

### Synthetic Procedures

As discussed hereinbefore, the detailed synthetic procedure for compounds of formula (I), (II) and (III) is described herein in Examples 1, 2 and 3, respectively.

Thus, according to a further aspect of the invention there is provided a process for preparing a compound of formula (I) as herein defined which comprises reacting a compound of formula (21) as described in Example 1, Scheme VI with a compound of formula (20) as described in Example 1, Scheme VI. Such a process typically comprises the use of a suitable base, such as trimethylamine and subjected to suitable reaction conditions, such as stirring for 24 h at room temperature.

According to a further aspect of the invention there is provided a process for preparing a compound of formula (II) as herein defined which comprises reacting a compound of formula (28) as described in Example 2, Scheme VII with a compound of formula (29) as described in Example 2, Scheme VII. Such a process typically comprises the use of a suitable base, such as trimethylamine and subjected to suitable reaction conditions, such as stirring for 24 h at room temperature.

According to a further aspect of the invention there is provided a process for preparing a compound of formula (III) as herein defined which comprises reacting a compound of formula (5) as described in Example 3, Scheme III with a compound of formula (8) as described in Example 3, Scheme III. Such a process typically comprises the use of a suitable base, such as trimethylamine and subjected to suitable reaction conditions, such as stirring for 2 h at room temperature.

### Natural Anti-Gal Antibody, α-Gal Epitope, and Xenograft Rejection

Anti-Gal is believed to be a natural antibody that may be present in all humans, constituting 0.1-2% of serum immunoglobulins (Bovin N. V., Biochemistry (Moscow), 2013; 78(7):786-797, Galili et al. J. Exp. Med. 1984; 160: 1519-31, and Hamadeh R M et al. Clin. Diagnos. Lab. Immunol. 1995; 2:125-31). Studies have presented data indicating that anti-Gal antibodies might interact specifically with α-Gal epitopes on cell surface or free glycolipids and glycoproteins. (Galili U et al. J. Exp. Med. 1985, 162: 573-82, and Galili U. Springer Semin Immunopathol. 1993; 15: 155-171). It is further reported that the anti-Gal antibody may be produced throughout life as a result of antigenic stimulation by bacteria of the gastrointestinal flora (Galili U et al. Infect. Immun. 1988; 56: 1730-37).

The α-Gal epitope can be abundantly bio-synthesized on glycolipids and glycoproteins by the glycosylation enzyme α1,3galactosyltransferase within the Golgi apparatus of cells of non-primate mammals, prosimians and in New World monkeys (Galili U et al. Biol. Chem. 1988; 263; 17755-62). In contrast, humans, apes, and Old World monkeys lack α-Gal epitopes, but produce the natural anti-Gal antibody in very large amounts (Galili U et al. Proc. Natl. Acad. Sci. USA 1987, 84: 1369-73). Based on the sequence of the α1,3galactosyltransferase pseudogene in monkeys and apes, it was estimated that the α1,3galactosyltransferase gene was inactivated in ancestral Old World primates approximately 20 million years ago (Galili U, Swanson K. Proc. Natl. Acad. Sci. USA 1991; 88: 7401-04). It was suggested that this evolutionary event was associated with the appearance of an infectious microbial agent, endemic to the Old World (i.e. currently Europe, Asia and Africa), which was detrimental to primates and which expressed α-Gal epitopes. Primates could produce anti-Gal as a protective antibody against such putative detrimental agent, only after they evolved under a selective pressure for the inactivation of the α1,3galactosyltransferase gene and thus, loss of immune tolerance to the α-Gal epitope (Galili U, Andrews P. J. Human Evolution 29:433-42, 1995).

The strong protective activity of the natural anti-Gal antibody has been evolutionarily conserved in humans and monkeys. This can be inferred from xenotransplantation studies with pig organs expressing α-Gal epitopes. Since cells of various mammals, including pigs, express α-Gal epitopes, organs from pigs transplanted in humans, or in Old World monkeys, are rejected because of the in vivo binding of the anti-Gal antibody to these epitopes on pig cells (Galili, U. Immunol. Today 1993, 14: 480-82). Transplantation of pig tissues into humans or into Old World monkeys results in avid anti-Gal binding to α-Gal epitopes on an in vivo graft and the subsequent induction of the xenograft rejection. Vascularized xenografts (e.g. pig heart) undergo rapid rejection (called hyperacute rejection) in monkeys within 30-60 minutes mostly as a result of anti-Gal antibody molecules binding to α-Gal epitopes on pig endothelial cells, activation of complement, lysis of the endothelial cells, and collapse of the vascular bed (Collins B H et al. J. Immunol. 1995; 154: 5500-10). In addition, much of the destruction of xenograft cells in extravascular areas is mediated by anti-Gal IgG binding to α-Gal epitopes on various cells. This binding results in antibody dependent cell mediated cytolysis (ADCC), following the binding of the Fc portion of anti-Gal IgG to cell bound Fcγ receptors on granulocytes, macrophages, and NK cells.

The anti-Gal mediated destruction of xenografts could be monitored with pig cartilage (an avascular xenograft tissue) transplanted into rhesus monkeys (i.e. monkeys that naturally produce anti-Gal antibodies). Studies indicate that the binding of anti-Gal to α-Gal epitopes in the pig tissue results in induction of an extensive inflammatory reaction that leads to gradual destruction of the tissue within 2 months (Stone K R et al. Transplantation 1998, 65: 1577-83). Binding of anti-Gal to α-Gal epitopes on the cartilage cellular and extracellular matrix glycoproteins further opsonizes them (i.e., forms immune complexes with them) and thus, targets them to antigen presenting cells by the binding of the Fc portion of the immuno-complexed anti-Gal to Fcγ receptors on antigen presenting cells. The antigen presenting cells, in turn, transport these pig glycoproteins to draining lymph nodes where they activate the many T cells specific to the multiple pig xenopeptides. These activated T cells subsequently migrate into the cartilage xenograft implant and comprise approximately 80% of the infiltrating mononuclear cells. That this inflammatory response is primarily mediated by anti-Gal interaction with α-Gal epitopes can be inferred from monitoring the immune response to the pig cartilage xenograft from which the α-Gal epitopes were removed by an enzymatic treatment (for example, using recombinant α-Galactosidase). α-Galactosidase destroys the α-Gal epitopes on the cartilage glycoproteins by cleaving (hydrolyzing) the terminal α-Galactosyl unit. In the absence of α-Gal epitopes on the pig cartilage glycoproteins, there is no anti-Gal binding to the xenograft, and thus, no effective antigen presenting cell mediated transport of the xenoglycoproteins occurs. This is indicated by a lack of significant T cell infiltration in a xenograft.

The present invention contemplates exploiting the immunologic potential of the natural anti-Gal antibody, demonstrated in pig cartilage xenograft rejection, for the regression and/or destruction of tumour lesions, treated to display α-Gal epitopes and for targeting the tumour cell membranes to antigen presenting cells by anti-Gal antibody. It is believed that such treatment will convert the tumour lesions into in situ autologous tumour vaccines that elicit a systemic protective immune response against the metastatic tumour cells by similar mechanisms as those observed in rejection of pig cartilage in monkeys. It is further believed that the anti-Gal IgG molecules binding to tumour cells expressing α-Gal epitopes will target tumour cell membranes to antigen presenting cells for eliciting a protective anti-tumour immune response against the autologous tumour antigens expressed on the tumour cells in the treated lesion and also expressed on metastatic tumour cells.

### Pharmaceutical Compositions

According to a further aspect of the invention, there is provided a pharmaceutical composition comprising a glycolipid compound selected from a compound of formula (I), (II) and (III) or a pharmaceutically acceptable salt thereof as defined herein.

According to a further aspect of the invention, there is provided a glycolipid compound selected from a compound of formula (I), (II) and (III) or a pharmaceutically acceptable salt thereof as defined herein or a pharmaceutical composition as defined herein for use in the treatment of a tumour.

In one embodiment, the tumour is a solid tumour, myeloma, or a lymphoma. In a further embodiment, the tumour is a solid tumour. In an alternative embodiment, the tumour is a non-solid tumour.

In one embodiment, the tumour is a tumour originating from an organ selected from peritoneum, liver, pancreas, lung, urinary bladder, prostate, uterus, cervix, vagina, bone marrow, breast, skin, brain, lymph node, head and neck, stomach, intestine, colon, kidney, testis, and ovaries.

In one embodiment, the tumour comprises a primary tumour and/or a metastasis. In a further embodiment, the tumour comprises a primary tumour. In an alternative embodiment, the tumour comprises a secondary tumour.

In one embodiment, the tumour comprises melanoma, sarcoma, glioma, or carcinoma cells. In a further embodiment, the tumour comprises melanoma or carcinoma cells, or a metastasis.

The composition may be prepared as an aqueous glycolipid preparation comprising the glycolipid compound of formula (I), (II) or (III), wherein said preparation comprises glycolipid micelles.

In one embodiment, the composition additionally comprises one or more pharmaceutically acceptable carrier(s), diluent(s) and/or excipient(s). The carrier, diluent and/or excipient must be “pharmaceutically acceptable” in the sense of being compatible with the other ingredients of the composition and not deleterious to the recipient thereof. The person skilled in the art will appreciate aspects of pharmaceutical formulation which are exemplified for instance in Remington: The Science and Practice of Pharmacy; Pharmaceutical Press; 22^{nd} Edition; Allen, Loyd V. Ed. 2012, London, UK.

The composition of the invention may be prepared by combining the glycolipid compound of formula (I), (II) or (III) with standard pharmaceutical carriers or diluents according to conventional procedures well known in the art. These procedures may involve mixing, granulating and compressing or dissolving the ingredients as appropriate to the desired preparation.

In one embodiment, the pharmaceutical composition may also contain deoxycholate, or other mild detergents that may increase penetration of the glycolipids into cell membranes.

The pharmaceutical compositions of the invention may be formulated for administration by any route, and include those in a form adapted for oral, topical or parenteral administration to mammals including humans.

Therefore, in one embodiment, the composition is for administration by injection. In an alternative embodiment, the composition is a topical application, such as a topical ointment, topical lotion or topical solution.

In one embodiment, the composition is administered in one dose or multiple doses, such as multiple doses. In a further embodiment, the multiple doses are administered simultaneously (i.e. on one occasion). In a further alternative embodiment, the multiple doses are administered sequentially (i.e. on two or more separate occasions, such as during separate treatments).

When administration is sequential (i.e. on separate occasions), the composition may be administered when suitable time has elapsed between administrations, for example, 3 days, 5 days, a week, two weeks, a month, 2 months, 3 months, 6 months, or 12 months.

For parenteral administration, fluid unit dosage forms are prepared utilising the composition and a sterile vehicle, such as water. In preparing solutions the composition can be dissolved in water for injection and filter-sterilised before filling into a suitable vial or ampoule and sealing.

The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives and emollients in ointments and creams.

The formulations may also contain compatible conventional carriers, such as cream or ointment bases and ethanol or oleyl alcohol for lotions.

### Combinations

It will be appreciated that the compound of the invention can be administered as the sole therapeutic agent or it can be administered in combination therapy with one of more other compounds (or therapies) for treatment of a tumour.

Thus, according to a further aspect of the invention there is provided a pharmaceutical composition comprising a glycolipid compound selected from a compound of formula (I), (II) and (III) or a pharmaceutically acceptable salt thereof as defined herein in combination with one or more additional therapeutic agents.

For the treatment of a tumour, the compound of the invention may be advantageously employed in combination with one or more other medicinal agents, more particularly, with one or more anti-cancer agents or adjuvants (supporting agents in the therapy) in cancer therapy.

Examples of other therapeutic agents or treatments that may be administered together (whether concurrently or at different time intervals) with the compounds of the invention include but are not limited to:
Topoisomerase I inhibitors;
Antimetabolites;
Tubulin targeting agents;
DNA binder and topoisomerase II inhibitors;
Alkylating Agents;
Monoclonal Antibodies;
Anti-Hormones;
Signal Transduction Inhibitors;
Proteasome Inhibitors;
DNA methyl transferases;
Cytokines and retinoids;
Chromatin targeted therapies;
Radiotherapy; and
Other therapeutic or prophylactic agents.

Particular examples of anti-cancer agents or adjuvants (or salts thereof), include but are not limited to any of the agents selected from groups (i)-(xlvi), and optionally group (xlvii), below:
(i) Platinum compounds, for example cisplatin (optionally combined with amifostine), carboplatin or oxaliplatin;
(ii) Taxane compounds, for example paclitaxel, paclitaxel protein bound particles (Abraxane™), docetaxel, cabazitaxel or larotaxel;
(iii) Topoisomerase I inhibitors, for example camptothecin compounds, for example camptothecin, irinotecan(CPT11), SN-38, or topotecan;
(iv) Topoisomerase II inhibitors, for example anti-tumour epipodophyllotoxins or podophyllotoxin derivatives for example etoposide, or teniposide;
(v) Vinca alkaloids, for example vinblastine, vincristine, liposomal vincristine (Onco-TCS), vinorelbine, vindesine, vinflunine or vinvesir;
(vi) Nucleoside derivatives, for example 5-fluorouracil (5-FU, optionally in combination with leucovorin), gemcitabine, capecitabine, tegafur, UFT, S1, cladribine, cytarabine (Ara-C, cytosine arabinoside), fludarabine, clofarabine, or nelarabine;
(vii) Antimetabolites, for example clofarabine, aminopterin, or methotrexate, azacitidine, cytarabine, floxuridine, pentostatin, thioguanine, thiopurine, 6-mercaptopurine, or hydroxyurea (hydroxycarbamide);
(viii) Alkylating agents, such as nitrogen mustards or nitrosourea, for example cyclophosphamide, chlorambucil, carmustine (BCNU), bendamustine, thiotepa, melphalan, treosulfan, lomustine (CCNU), altretamine, busulfan, dacarbazine, estramustine, fotemustine, ifosfamide (optionally in combination with mesna), pipobroman, procarbazine, streptozocin, temozolomide, uracil, mechlorethamine, methylcyclohexylchloroethylnitrosurea, or nimustine (ACNU);
(ix) Anthracyclines, anthracenediones and related drugs, for example daunorubicin, doxorubicin (optionally in combination with dexrazoxane), liposomal formulations of doxorubicin (eg. Caelyx™, Myocet™, Doxil™), idarubicin, mitoxantrone, epirubicin, amsacrine, or valrubicin;
(x) Epothilones, for example ixabepilone, patupilone, BMS-310705, KOS-862 and ZK-EPO, epothilone A, epothilone B, desoxyepothilone B (also known as epothilone D or KOS-862), aza-epothilone B (also known as BMS-247550), aulimalide, isolaulimalide, or luetherobin;
(xi) DNA methyl transferase inhibitors, for example temozolomide, azacytidine or decitabine;
(xii) Antifolates, for example methotrexate, pemetrexed disodium, or raltitrexed;
(xiii) Cytotoxic antibiotics, for example antinomycin D, bleomycin, mitomycin C, dactinomycin, carminomycin, daunomycin, levamisole, plicamycin, or mithramycin;
(xiv) Tubulin-binding agents, for example combrestatin, colchicines or nocodazole;
(xv) Signal Transduction inhibitors such as Kinase inhibitors (e.g. EGFR (epithelial growth factor receptor) inhibitors, VEGFR (vascular endothelial growth factor receptor) inhibitors, PDGFR (platelet-derived growth factor receptor) inhibitors, MTKI (multi target kinase inhibitors), Raf inhibitors, mTOR inhibitors for example imatinib mesylate, erlotinib, gefitinib, dasatinib, lapatinib, dovotinib, axitinib, nilotinib, vandetanib, vatalinib, pazopanib, sorafenib, sunitinib, temsirolimus, everolimus (RAD 001), or vemurafenib (PLX4032/RG7204);
(xvi) Aurora kinase inhibitors for example AT9283, barasertib (AZD1152), TAK-901, MK0457 (VX680), cenisertib (R-763), danusertib (PHA-739358), alisertib (MLN-8237), or MP-470;
(xvii) CDK inhibitors for example AT7519, roscovitine, seliciclib, alvocidib (flavopiridol), dinaciclib (SCH-727965), 7-hydroxy-staurosporine (UCN-01), JNJ-7706621, BMS-387032 (a.k.a. SNS-032), PHA533533, PD332991, ZK-304709, or AZD-5438;
(xviii) PKA/B inhibitors and PKB (akt) pathway inhibitors for example AT13148, AZ-5363, Semaphore, SF1126 and MTOR inhibitors such as rapamycin analogues, AP23841 and AP23573, calmodulin inhibitors (forkhead translocation inhibitors), API-2/TCN (triciribine), RX-0201, enzastaurin HCl (LY317615), NL-71-101, SR-13668, PX-316, or KRX-0401 (perifosine/NSC 639966);
(xix) Hsp90 inhibitors for example AT13387, herbimycin, geldanamycin (GA), 17-allylamino-17-desmethoxygeldanamycin (17-AAG) e.g. NSC-330507, Kos-953 and CNF-1010, 17-dimethylaminoethylamino-17-demethoxygeldanamycin hydrochloride (17-DMAG) e.g. NSC-707545 and Kos-1022, NVP-AUY922 (VER-52296), NVP-BEP800, CNF-2024 (BIIB-021 an oral purine), ganetespib (STA-9090), SNX-5422 (SC-102112) or IPI-504;
(xx) Monoclonal Antibodies (unconjugated or conjugated to radioisotopes, toxins or other agents), antibody derivatives and related agents, such as anti-CD, anti-VEGFR, anti-HER2 or anti-EGFR antibodies, for example rituximab (CD20), ofatumumab (CD20), ibritumomab tiuxetan (CD20), GA101 (CD20), tositumomab (CD20), epratuzumab (CD22), lintuzumab (CD33), gemtuzumab ozogamicin (CD33), alemtuzumab (CD52), galiximab (CD80), trastuzumab (HER2 antibody), pertuzumab (HER2), trastuzumab-DM1 (HER2), ertumaxomab (HER2 and CD3), cetuximab (EGFR), panitumumab (EGFR), necitumumab (EGFR), nimotuzumab (EGFR), bevacizumab (VEGF), ipilimumab (CTLA4), catumaxumab (EpCAM and CD3), abagovomab (CA125), farletuzumab (folate receptor), elotuzumab (CS1), denosumab (RANK ligand), figitumumab (IGF1R), CP751,871 (IGF1R), mapatumumab (TRAIL receptor), metMAB (met), mitumomab (GD3 ganglioside), naptumomab estafenatox (5T4), or siltuximab (IL6);
(xxi) Estrogen receptor antagonists or selective estrogen receptor modulators (SERMs) or inhibitors of estrogen synthesis, for example tamoxifen, fulvestrant, toremifene, droloxifene, faslodex, or raloxifene;
(xxii) Aromatase inhibitors and related drugs, such as exemestane, anastrozole, letrazole, testolactone aminoglutethimide, mitotane or vorozole;
(xxiii) Antiandrogens (i.e. androgen receptor antagonists) and related agents for example bicalutamide, nilutamide, flutamide, cyproterone, or ketoconazole;
(xxiv) Hormones and analogues thereof such as medroxyprogesterone, diethylstilbestrol (a.k.a. diethylstilboestrol) or octreotide;
(xxv) Steroids for example dromostanolone propionate, megestrol acetate, nandrolone (decanoate, phenpropionate), fluoxymestrone or gossypol,
(xxvi) Steroidal cytochrome P450 17alpha-hydroxylase-17,20-lyase inhibitor (CYP17), e.g. abiraterone;
(xxvii) Gonadotropin releasing hormone agonists or antagonists (GnRAs) for example abarelix, goserelin acetate, histrelin acetate, leuprolide acetate, triptorelin, buserelin, or deslorelin;
(xxviii) Glucocorticoids, for example prednisone, prednisolone, dexamethasone;
(xxix) Differentiating agents, such as retinoids, rexinoids, vitamin D or retinoic acid and retinoic acid metabolism blocking agents (RAMBA) for example accutane, alitretinoin, bexarotene, or tretinoin;
(xxx) Farnesyltransferase inhibitors for example tipifarnib;
(xxxi) Chromatin targeted therapies such as histone deacetylase (HDAC) inhibitors for example sodium butyrate, suberoylanilide hydroxamide acid (SAHA), depsipeptide (FR 901228), dacinostat (NVP-LAQ824), R306465/JNJ-16241199, JNJ-26481585, trichostatin A, vorinostat, chlamydocin, A-173, JNJ-MGCD-0103, PXD-101, or apicidin;
(xxxii) Proteasome Inhibitors for example bortezomib, carfilzomib, CEP-18770, MLN-9708, or ONX-0912;
(xxxiii) Photodynamic drugs for example porfimer sodium or temoporfin;
(xxxiv) Marine organism-derived anticancer agents such as trabectidin;
(xxxv) Radiolabelled drugs for radioimmunotherapy for example with a beta particle-emitting isotope (e.g., Iodine-131, Yittrium-90) or an alpha particle-emitting isotope (e.g., Bismuth-213 or Actinium-225) for example ibritumomab or Iodine tositumomab;
(xxxvi) Telomerase inhibitors for example telomestatin;
(xxxvii) Matrix metalloproteinase inhibitors for example batimastat, marimastat, prinostat or metastat;
(xxxviii) Recombinant interferons (such as interferon-γ and interferon α) and interleukins (e.g. interleukin 2), for example aldesleukin, denileukin diftitox, interferon alfa 2a, interferon alfa 2b, or peginterferon alfa 2b;
(xxxix) Selective immunoresponse modulators for example thalidomide, or lenalidomide;
(xl) Therapeutic Vaccines such as sipuleucel-T (Provenge) or OncoVex;
(xli) Cytokine-activating agents include Picibanil, Romurtide, Sizofiran, Virulizin, or Thymosin;
(xlii) Arsenic trioxide;
(xliii) Inhibitors of G-protein coupled receptors (GPCR) for example atrasentan;
(xliv) Enzymes such as L-asparaginase, pegaspargase, rasburicase, or pegademase;
(xlv) DNA repair inhibitors such as PARP inhibitors for example, olaparib, velaparib, iniparib, INO-1001, AG-014699, or ONO-2231;
(xlvi) Agonists of Death receptor (e.g. TNF-related apoptosis inducing ligand (TRAIL) receptor), such as mapatumumab (formerly HGS-ETR1), conatumumab (formerly AMG 655), PRO95780, lexatumumab, dulanermin, CS-1008, apomab or recombinant TRAIL ligands such as recombinant Human TRAIL/Apo2 Ligand;
(xlvii) Prophylactic agents (adjuncts); i.e. agents that reduce or alleviate some of the side effects associated with chemotherapy agents, for example
   anti-emetic agents,
   agents that prevent or decrease the duration of chemotherapy-associated neutropenia and prevent complications that arise from reduced levels of platelets, red blood cells or white blood cells, for example interleukin-11 (e.g. oprelvekin), erythropoietin (EPO) and analogues thereof (e.g. darbepoetin alfa), colony-stimulating factor analogs such as granulocyte macrophage-colony stimulating factor (GM-CSF) (e.g. sargramostim), and granulocyte-colony stimulating factor (G-CSF) and analogues thereof (e.g. filgrastim, pegfilgrastim),
   agents that inhibit bone resorption such as denosumab or bisphosphonates e.g. zoledronate, zoledronic acid, pamidronate and ibandronate,
   agents that suppress inflammatory responses such as dexamethasone, prednisone, and prednisolone,
   agents used to reduce blood levels of growth hormone and IGF-I (and other hormones) in patients with acromegaly or other rare hormone-producing tumours, such as synthetic forms of the hormone somatostatin e.g. octreotide acetate,
   antidote to drugs that decrease levels of folic acid such as leucovorin, or folinic acid,
   agents for pain e.g. opiates such as morphine, diamorphine and fentanyl,
   non-steroidal anti-inflammatory drugs (NSAID) such as COX-2 inhibitors for example celecoxib, etoricoxib and lumiracoxib,
   agents for mucositis e.g. palifermin,
   agents for the treatment of side-effects including anorexia, cachexia, oedema or thromoembolic episodes, such as megestrol acetate.

In one particular embodiment, the pharmaceutical composition additionally comprises one or more systemic inhibitors of immune system down-regulation. Examples of suitable systemic inhibitors of immune system down-regulation are described in US 2012/263677 and include anti-CTLA-4, anti-PD-1 and anti-PD-L1 antibodies.

In a yet further embodiment, the one or more systemic inhibitors of immune system down-regulation are selected from anti-PD-1 antibodies.

In a further embodiment, the pharmaceutical composition additionally comprises one or more enhancers of immune system up-regulation. Examples of suitable enhancers of immune system up-regulation are described in US 2012/263677 and include suitable non-specific cytokines, such as interleukin-1, -2, or -6 (IL-1, IL-2 or IL-6) and aldesleukin; interferon-alpha or gamma (IFN-α and IFN-γ), interferon alfa-2b and pegylated interferon (including pegylated interferon alfa-2a and pegylated interferon alfa-2b); granulocyte macrophage colony stimulating factor (GM-CSF, molgramostim or sargramostim); dendritic cell vaccines and other allogeneic or autologous therapeutic cancer vaccines, including intralesional vaccines containing an oncolytic herpes virus encoding GM-CSF (OncoVex®) or a plasmid encoding human leukocyte antigen-B7 and beta-2 microglobulin agent designed to express allogeneic MHC class I antigens (Allovectin-7®); and antibodies against specific tumour antigens. In a yet further embodiment, the one or more enhancers of immune system up-regulation are selected from IL-2 and interferon-gamma.

Each of the compounds present in the combinations of the invention may be given in individually varying dose schedules and via different routes. For example, the glycolipid compounds of the invention are intended to be administered directly to the tumour whereas the systemic inhibitors of immune system down-regulation, such as anti-PD-1 antibodies, will typically be delivered systemically, i.e. by intravenous injection. As such, the posology of each of the two or more agents may differ: each may be administered at the same time or at different times. A person skilled in the art would know through his or her common general knowledge the dosing regimes and combination therapies to use. For example, the compound of the invention may be using in combination with one or more other agents which are administered according to their existing combination regimen.

### Methods of Treatment

According to a further aspect of the invention, there is provided a method of treating a tumour in a subject, comprising:
a) providing:
   i) a subject comprising at least one tumour that comprises a plurality of cancer cells having a cell surface; and
   ii) the glycolipid compound selected from a compound of formula (I), (II) and (III) or a pharmaceutically acceptable salt thereof or the pharmaceutical composition as defined herein; and
b) introducing said glycolipid or composition into the tumour.

In one embodiment, the glycolipid or pharmaceutical composition induces an immune response to the tumour thereby treating the tumour.

In one embodiment, the invention provides a method for inducing an immune response to a tumour in a subject, comprising:
a) administering to a subject comprising at least one tumour, an effective amount of a glycolipid compound selected from a compound of formula (I), (II) and (III) or a pharmaceutically acceptable salt thereof or the pharmaceutical composition as defined herein to induce an immune response to the at least one tumour.

In one embodiment, the invention provides a method for treating a tumour in a subject, comprising:
a) administering to a subject comprising at least one tumour, an effective amount of a glycolipid compound selected from a compound of formula (I), (II) and Op or a pharmaceutically acceptable salt thereof or the pharmaceutical composition as defined herein to induce an immune response to the at least one tumour,
wherein inducing an immune response to the tumour results in a reduction in the tumour thereby treating the tumour in the subject.

In one embodiment, the composition further comprises at least one systemic inhibitor of immune system down-regulation.

In one embodiment, the at least one systemic inhibitor of immune system down-regulation is selected from anti-CTLA-4, anti-PD-1 and anti-PD-L1 antibodies.

In one embodiment, the method is repeated 1-5 times until the tumour is reduced in size.

In one embodiment, the method is repeated 1-5 times until the tumour is undetectable.

In one embodiment, the glycolipid or pharmaceutical composition is injected into a primary tumour and induces an immune response that is effective in treating at least one secondary tumour that arose from the primary tumour.

In one embodiment, the glycolipid or pharmaceutical composition is injected into a primary tumour, and induces an immune response that is effective in reducing the size of at least one secondary tumour that arose from the primary tumour.

In one embodiment, the method further comprises surgical removal of the tumour after inducing an immune response to the tumour.

In one embodiment, the method further comprises surgical removal of the tumour after administration of the glycolipid or pharmaceutical composition.

In one embodiment, the surgical removal of the tumour occurs between about 1-21 days after administration of the glycolipid or pharmaceutical composition.

In one embodiment, the surgical removal of the tumour occurs between about 1-14 days after administration of the glycolipid or pharmaceutical composition.

In one embodiment, the surgical removal of the tumour occurs between about 1-7 days after administration of the glycolipid or pharmaceutical composition.

In one embodiment, the surgical removal of the tumour occurs between about 7-14 days after administration of the glycolipid or pharmaceutical composition.

In one embodiment, the surgical removal of the tumour occurs between about 14-21 days after administration of the glycolipid or pharmaceutical composition.

The method of the invention allows for the administration of the glycolipid compound of the invention in order to display an α-Gal or GalNAc epitope on the cell surface of the cancer cells.

In one embodiment, the method further comprises displaying a membrane-bound α-Gal or

GalNAc epitope on said tumour cell.

In one embodiment, the present invention contemplates a method of treating a subject, comprising:
a) providing:
   i) a subject having endogenous anti-Gal or anti-GalNAc antibody and a plurality of nonresectable tumours, wherein at least a subset of said tumours is accessible via a procedure selected from the group consisting of direct injection, injection by endoscopy, bronchoscopy, cystoscopy, colonoscopy, laparoscopy, and catheterization,
   ii) the glycolipid compound or pharmaceutical composition as defined herein; and
b) intratumourally injecting said glycolipid compound or composition using said procedure.

In one embodiment, the α-Gal or GalNAc epitope of the glycolipid compounds of the invention becomes opsonized. In one embodiment, the opsonized α-Gal or GalNAc epitope induces production of an autologous vaccine against said tumour by targeting tumour cells and cell membranes to antigen presenting cells.

In one embodiment, the subject is a human or a mouse. In one embodiment, the subject is a human. In an alternative embodiment, the subject is a mouse.

According to another aspect of the invention, there is provided a method of introducing the glycolipid compounds of the invention into a tumour in a mouse, comprising:
a) providing:
   i) a mouse, (1) lacking an α1,3galactosyltransferase gene, (2) having anti-Gal antibodies, and (3) comprising at least one tumour comprising a plurality of cancer cells having a cell surface; and
   ii) a glycolipid compound selected from a compound of formula (I) and (II) or a pharmaceutically acceptable salt thereof; and
b) introducing said glycolipid into at least one of said tumours to display an α-Gal epitope on the cell surface of the cancer cells.

### Anti-Gal Targeting of Autologous Tumour Vaccines to Antigen Presenting Cells

It has been shown that α-Gal epitopes can be inserted in vitro into a tumour cell membrane by incubation of tumour cells with α-Gal glycolipids. The co-incubation of tumour cells or tumour cell membranes with such α-Gal glycolipids results in their spontaneous in vitro insertion into the tumour cell membranes and the expression of α-Gal epitopes on these cell membranes. Tumour cells engineered to express α-Gal epitopes by various molecular biology methods with the α1,3galactosyltransferase gene were studied as autologous tumour vaccines. Following their intradermal injection, the natural anti-Gal IgG antibody binds in situ at the vaccination site, to the α-Gal epitopes on the vaccinating tumour cell membrane and target the vaccine to antigen presenting cells. Although it is not necessary to understand the mechanism of an invention, it is believed that the binding of the Fc portion of the complexed anti-Gal to Fcγ receptors on antigen presenting cells induces effective uptake of the opsonized vaccinating tumour cell membranes into antigen presenting cells. Thus, the uncharacterized tumour antigens of the autologous tumour are also internalized into the antigen presenting cells. After transport of vaccinating autologous tumour membranes to the draining lymph nodes, the antigen presenting cells process and present the tumour antigen peptides for activation of tumour specific cytotoxic and helper T cells (i.e., CD8⁺ and CD4⁺ T cells, respectively).

A proof of principle for the efficacy of tumour vaccines expressing α-Gal epitopes was achieved in studies in a mouse experimental model immunized with melanoma cells expressing α-Gal epitopes and challenged with the same melanoma cells which, however, lack α-Gal epitopes (LaTemple D C et al. Cancer Res. 1999, 59: 3417-23, and Deny L et al. Cancer Gene Therapy 2005; 12: 528-39). The mice used in those studies were knockout mice for the α1,3galactosyltransferase gene (i.e., these mice lack the α-Gal epitope and can produce the anti-Gal antibody). Mice immunized with melanoma cells engineered to express α-Gal epitopes displayed an effective immune protection against challenge with the same tumour cells, which however lack α-Gal epitopes. In contrast, mice immunized with tumour cells lacking α-Gal epitopes, did not display a protective immune response against challenge with the live tumour cells lacking α-Gal epitopes.

### α-Gal Glycolipids in Tumour Therapy

The present invention contemplates the treatment of patients with solid tumour masses. Particular embodiments of the present invention contemplate novel immunotherapy treatments of cancer patients that aim to immunize the individual patient against his or her own tumour lesions by conversion of the patient's own tumour into an autologous tumour vaccine (see U.S. Pat. No. 5,879,675, herein incorporated by reference). For example, the '675 patent teaches an in vitro processing of tumour cells and/or cell membranes. Upon injection of these cells into a patient the vaccine is targeted by anti-Gal antibody to APCs and elicits a protective immune response against an autologous tumour antigen. Unlike the present invention, however, the '675 patent does not teach: i) an in vivo intratumoural treatment for the induction of inflammation, regression and/or destruction of the tumour by the natural anti-Gal antibody; or
ii) the display of α-Gal epitopes on tumour cells in vivo following an intratumoural injection of α-Gal glycolipids within cancer patients.

In one embodiment of the present invention α-Gal glycolipids may be delivered into a tumour lesion comprising tumour cells by a non-surgical intratumoural injection (i.e., for example, by endoscopy, catheterization, or the like), or by any other method for in vivo introduction into tumours of the α-Gal glycolipids, or anti-Gal binding epitopes on various molecules.

Post-surgery recurrence of chemotherapy refractory metastases, is believed to be the most common cause of death in patients with solid tumours. High incidence of such relapsing metastases (80%) have been reported in patients with pancreatic and ovarian carcinomas and to a somewhat lesser extent in other solid tumours such as melanoma and colorectal, lung and breast carcinoma. Many of these relapsing patients are considered to have terminal disease, as no treatment is available for them, and they die within weeks or months after detection of the metastases.

In one embodiment, the present invention contemplates a therapeutic method for regression and/or destruction of tumour metastases by exploiting the fact that all humans, naturally produce the anti-Gal antibody as approximately 1% of their immunoglobulins. The immunological potential of the anti-Gal antibody can be harnessed to regress and/or destroy any tumour lesions and converting them into an in situ autologous tumour vaccine by intratumoural injection of glycolipids carrying the α-Gal epitope (i.e. the glycolipid compounds of formula (I) or (II)).

Therefore, the invention described herein may induce regression and/or destruction of the treated tumour lesions. Thus, in one embodiment, the treated tumour undergoes regression. In an alternative embodiment, the treated tumour is destroyed.

In a further embodiment, the tumour (i.e. which is displaying the α-Gal epitope) undergoes regression, wherein said tumour is selected from a melanoma or an organ metastasis, such as liver metastasis. In a further alternative embodiment, the tumour (i.e. which is displaying the α-Gal epitope) is destroyed, wherein said tumour is selected from a melanoma or an organ metastasis, such as liver metastasis.

In one embodiment, the introducing step causes regression of a second tumour in the subject as a result of the conversion of the treated tumour into an autologous tumour vaccine. In a further embodiment, said second tumour is selected from a melanoma or a liver metastasis.

In one embodiment, the introducing step causes destruction of a second tumour in the subject. In a further embodiment, said second tumour is selected from a melanoma or a liver metastasis.

Many α-Gal glycolipids will spontaneously insert into the tumour cell membranes, since the hydrophobic (i.e. lipophilic) lipid tail of the α-Gal glycolipids is in a more stable energetic form when embedded in the outer leaflet of the lipid bilayer of the cell membrane as compared to a water-surrounded micellular core. Spontaneous insertion (incorporation) of other types of glycolipids called gangliosides into cell membranes has been previously demonstrated (Kanda S et al. J Biochem. (Tokyo). 1982; 91: 1707-18, and Spiegel S et al. J. Cell Biol. 1985; 100: 721-26). The insertion of α-Gal glycolipids into the tumour cell membranes is expected to result in the de novo display of α-Gal epitopes on the cell membrane surface. α-Gal epitope expression may facilitate an anti-Gal antibody mediated regression and/or destruction of the tumour cells by such mechanisms which include, but are not limited to, complement mediated cytolysis (CDC) and antibody dependent cell mediated cytolysis (ADCC) and may also lead to tumour necrosis. An anti-Gal opsonized tumour cell membrane will then be effectively targeted by antigen presenting cells, thereby converting the treated tumour lesions into autologous tumour vaccines. This autologous vaccine will then stimulate the immune system to react against tumour antigens resulting in the further regression and/or destruction of tumour cells expressing these antigens within other tumour lesions and/or micrometastases of the treated patient.

In one embodiment, the subject was treated previously to surgically remove the tumour.

In an alternative embodiment, the subject was not treated previously to surgically remove the tumour, i.e., the method described herein may be performed as neo-adjuvant therapy several weeks prior to resection of the primary tumour. In one embodiment, an intratumoural injection of the glycolipids of the invention decreases the size of the tumour and converts the treated tumour into an autologous tumour vaccine. Although such a tumour will be eventually resected, it is believed that prior to its resection the treated tumour will elicit an immune response against micrometastases that display the same tumour antigens.

Mechanisms of Anti-Gal Antibody Tumour Regression and/or Destruction

Although it is not necessary to understand the mechanism of an invention, it is believed that tumour lesion regression and/or destruction by the injected α-Gal glycolipids may comprise a biochemical and physiological basis.

In one embodiment, the method further comprises inducing an intratumoural inflammation.

An intratumoural injection may result in a local rupture of tumour associated capillaries thereby providing natural anti-Gal IgM and anti-Gal IgG antibody molecules access to the tumour interior. Anti-Gal antibodies would then be able to interact with the α-Gal epitopes on α-Gal glycolipid micelles, or individual α-Gal glycolipids molecules, thereby inducing local activation of complement and generation of the complement cleavage chemotactic factors C5a and C3a. Moreover, C3b gets covalently deposited onto target cells. Complement activation then initiates a local inflammatory process facilitating intratumoural granulocytes, monocytes, macrophages and dendritic cell migration directed by the de novo produced C5a and C3a chemotactic factors within the treated tumour lesions. The inflammatory process may be further amplified as a result of the insertion of α-Gal glycolipids into cell membranes causing an anti-Gal activation of endothelial cells (Palmetshofer A et al. Transplantation. 1998; 65: 844-53; Palmetshofer A et al. Transplantation. 1998; 65: 971-8). Endothelial cell activation and overall tumour cell damage may result in local production of additional pro-inflammatory cytokines and chemokines. These locally secreted cytokines and chemokines induce additional migration of macrophages, dendritic cells, and subsequent migration of lymphocytes into the lesion injected with α-Gal glycolipids. This cellular migration is mediated by receptors to pro-inflammatory cytokines and chemokines on antigen presenting cells and on lymphocytes (Cravens P D and Lipsky P E Immunol. Cell Biol. 2002; 80: 497-505). This initial induction of an inflammatory response enables the immune system to overcome its general lack of ability to detect the “stealthy nature” of developing tumour lesions. This inflammation also enables the immune system to overcome the immunosuppressive microenvironment within solid tumour lesions that is induced by the local cytokine milieu, and which normally prevent lymphocytes from penetrating into the tumour (Malmberg K J. Cancer Immunol. Immunother. 2004; 53: 879-92; Lugade A A et al. J. Immunol. 2005; 174:7516-23).

Destruction of the tumour cells occurs by anti-Gal binding to α-Gal glycolipids inserted into cell membranes. α-Gal glycolipids injected into a tumour may spontaneously insert into the outer leaflet of the phospholipid bilayer of tumour cell membranes. The subsequent binding of anti-Gal IgM and/or anti-Gal IgG to the α-Gal epitopes on the inserted α-Gal glycolipid induces the regression and/or destruction of the treated tumour via complement dependent cytolysis (CDC). The binding of anti-Gal IgG molecules to these α-Gal epitopes also facilitates antibody dependent cell cytolysis (ADCC) of the tumour cells.

In one embodiment, the tumour undergoes regression and/or destruction via complement dependent cytolysis (CDC).

In one embodiment, the tumour undergoes regression and/or destruction via antibody dependent cell cytolysis (ADCC).

In complement dependent cytolysis, it is believed that anti-Gal IgG and/or IgM molecules binding to tumour cells expressing α-Gal epitopes (due to α-Gal glycolipid insertion) activate the complement system. Subsequently, the complement C5b-9 membrane attack complex is formed as a result of this complement activation, then “pokes” holes in the tumour cell membranes, resulting in tumour cell lysis. This complement dependent cytolysis is similarly found when pig endothelial cells are lysed, leading to hyperacute rejection of xenografts (Collins B H et al. J. Immunol. 1995; 154: 5500-10,). In ADCC the effector cells are granulocytes, macrophages, and NK cells. These cells are attracted to the lesion because of the anti-Gal induced inflammatory process. They bind via their Fcγ receptors (FcγR) to the Fc portion of anti-Gal IgG molecules which are bound to the α-Gal glycolipid inserted into the tumour cell membrane. Once attached to the tumour cells, these effector cells secrete their granzyme vesicles into the membrane contact areas generating holes in the tumour cell membrane, thus inducing the destruction of these tumour cells. The efficacy of anti-Gal IgG in inducing ADCC destruction of cells expressing α-Gal epitopes was demonstrated with xenograft pig cells binding anti-Gal via their α-Gal epitopes (Galili, U. Immunol. Today 1993, 14: 480-82). A similar anti-Gal mediated ADCC process occurs when tumour cells bind anti-Gal via α-Gal epitopes expressed on their cell surface membrane (Tanemura M et al. J. Clin. Invest. 2000; 105: 301-10).

The uptake of tumour cell membranes by antigen presenting cells may result in an induction of a protective immune response against autologous tumour antigens in order to regress and/or destroy chemotherapy refractive micrometastases. Anti-Gal IgG antibody bound to α-Gal epitopes on membrane inserted α-Gal glycolipids or C3b deposited on the target cells via anti-Gal dependent complement activation stimulates antigen presenting cells to internalize cell membranes expressing the tumour antigens (i.e., for example, tumour associated antigens, TAAs). The internalized tumour antigens can then be transported by the antigen presenting cells from the treated tumour lesion to the draining lymph nodes. These tumour antigens may then be further processed by the antigen presenting cells and presented as immunogenic tumour peptides that activate tumour specific T cells. This process results in the induction of a systemic protective anti-tumour immune response (i.e., for example, an autologous tumour vaccine). Therefore, tumour lesions injected with α-Gal glycolipids ultimately are converted into in situ autologous tumour vaccines that elicit an immune response against micrometastases expressing the tumour antigens as those in the treated tumour lesions.

As a clinical treatment modality, glycolipids can be administered into cancer lesions by various methods including, but not limited to, an intradermal injection (i.e., for example, into a melanoma tumour); an endoscopic injection (i.e., for example, into colorectal intestinal metastases); a laparoscopic injection (i.e., for example, into abdominal ovarian, colon, gastric, liver, or pancreatic carcinoma metastases (e.g. on the peritoneum or in the liver)); a transcutaneous imaging guided needle injection (i.e., for example, into lung tumours); bronchoscopic injection (i.e., for example, into lung tumours); colonoscopic injection; or a cystoscopic injection (i.e., for example, into urinary bladder carcinomas).

Therefore, in one embodiment, the introducing comprises a procedure including, but not limited to, injection, imaging guided injection, endoscopy, bronchoscopy, cystoscopy, colonoscopy, laparoscopy and catheterization.

In one embodiment, the introducing comprises non-surgical intratumoural injection. For example, the introducing comprises a procedure selected from: intradermal injection, transcutaneous imaging guided injection, endoscopic injection, bronchoscopic injection, cytoscopic injection, colonoscopic injection and laproscopic injection.

In one embodiment, the glycolipid of the invention is injected in a pharmaceutically acceptable solution (i.e. a sterile solution) selected from the group including, but not limited to, phosphate buffered saline (PBS), saline, other aqueous solutions or other excipients Generally Recognized As Safe (GRAS). In one embodiment, the solution of glycolipids may also contain deoxycholate, or other mild detergents that may increase penetration of the glycolipids into cell membranes.

In one embodiment, the present invention contemplates an intratumoural injection of the glycolipids of the invention into primary tumours as a neo-adjuvant therapy provided before tumour resection surgery. In one embodiment, a rapid inflammatory response induced by the pre-surgical injection by a glycolipid results in decreasing the tumour lesion size, as well as converting it into an in situ autologous tumour vaccine. Although it is not necessary to understand the mechanism of an invention, it is believed that the immune response to the treated tumour may ultimately help to induce the immune destruction of micrometastases that are not detectable at the time of surgical resection of primary tumours. It is further believed that pre-surgical administration may help in preventing recurrence of the disease due to immunological destruction of micrometastases resistant to conventional adjuvant therapy (i.e., for example, chemotherapy and radiation) and which express tumour antigens as does the primary tumour. Such neo-adjuvant therapy may be administered to any solid tumour or lymphoma that can be injected directly, or by guided imaging, or any other known method.

According to a further aspect of the invention, there is provided a kit comprising the pharmaceutical composition as defined herein, and optionally instructions to use said kit in accordance with the method as defined herein.

In one embodiment, the kit additionally comprises a delivery device, such as an intratumoural delivery device.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure.

The following examples are intended only as illustrative examples of embodiments of the invention. They are not to be considered as limiting the present invention.

### Materials and Methods

Acetone, benzene, chloroform, ethylacetate, methanol, o-xylene, toluene, 2-propanol and o-xylene were from Chimmed (Russian Federation). Acetonitrile was from Cryochrom (Russian Federation). DMSO, DMF, CF₃COOH, Et₃N, N,N′-dicyclohexylcarbodiimide and N-hydroxysuccinimide were from Merck (Germany). N-methylmorpholin (NMM), 2-maleimidopropionic acid and disuccimidilcarbonate were supplied by Fluka. Iminodiacetic acid dimethyl ester hydrochloride was from Reakhim (Russian Federation). Tetraamine (H₂N—CH₂)₄C×2H₂SO₄ was synthesized as described by Litherland and Mann (1938) The amino-derivatives of pentaerythritol Part I. Preparation Journal of the Chemical Society, 1588-95. Dowex 50λ4-400 and Sephadex LH-20 were from Amersham Biosciences AB (Sweden). Silica gel 60 was from Merck (Germany). Thin-layer chromatography was performed using silica gel 60 F₂₅₄ aluminium sheets (Merck, 1.05554) with detection by charring after 7% H₃PO₄ soaking or ninhydrin.

¹H NMR spectra were recorded at 30° C. with a Bruker WM 500 MHz instrument or Bruker DRX-500 spectrometer using the signal of the solvent's residual protons as reference ([D₆]DMSO, 2.500 ppm; [D₂]H₂O, 4.750 ppm; CD₃OD).

### Example 1: Preparation of the Compound of Formula (I) “Galili-CMG2-DOPE”

### Preparation of 3-trifluoroacetamidopropyl-3,4-di-O-acetyl-2,6-di-O-benzyl-α-D-galactopyranosyl-(1→3)-2,4-di-O-acetyl-6-O-benzyl-β-D-galactopyranosyl-(1→4)-2-acetamido-3-O-acetyl-6-O-benzyl-2-deoxy-β-D-glucopyranoside (3) (Scheme I)

The glycosyl acceptor (3-trifluoroacetamidopropyl)-2-acetamido-3-O-acetyl-6-O-benzyl-2-deoxy-4-O-(2,4-di-O-acetyl-6-O-benzyl-β-D-galactopyranosyl)-β-D-glucopyranoside (2) was prepared according to the method disclosed in the publication of Pazynina et al (2008). A mixture of the glycosyl acceptor 2 (500 mg, 0.59 mmol), thiogalactopyranoside 1 (576 mg, 1.18 mmol), NIS (267 mg, 1.18 mmol), anhydrous CH₂Cl₂ (25 ml) and molecular sieves 4 Å (500 mg) was stirred at −45° C. for 30 min under an atmosphere of Ar. A solution of TfOH (21 μl, 0.236 mmol) in anhydrous CH₂Cl₂ (0.5 ml) was then added. The reaction mixture was stirred for 2 h at −45° C. and the temperature was then increased to −20° C. over 4 h. The mixture was kept at −20° C. overnight. Then extra amounts of thiogalactopyranoside 1 (144 mg, 0.295 mmol), NIS (66 mg, 0.295 mmol) and TfOH (5 μl, 0.06 mmol) were added and the stirring maintained at −20° C. for 2 h before being allowed to slowly warm up to r.t. (1 h). A saturated aqueous solution of Na₂S₂O₃ was then added and the mixture filtered. The filtrate was diluted with CHCl₃ (300 ml), washed with H₂O (2×100 ml), dried by filtration through cotton wool, and concentrated. Gel filtration on LH-20 (CHCl₃-MeOH) afforded the product 3 (600 mg, 80%), as a white foam.

¹H NMR (700 MHz, CDCl₃, characteristic signals), δ, ppm: 1.78-1.82 (m, 4H, CHCHC, OC(O)CH₃), 1.84-1.90 (m, 1H, CHCHC), 1.91, 1.94, 1.97, 1.98, 2.06 (5 s, 5×3H, 4 OC(O)CH₃, NH(O)CH₃), 3.23-3.30 (m, 1H, NCHH), 3.59-3.65 (m, 1H, NCHH), 4.05 (m, 1H, H-2^{I}), 4.33 (d, 1H, J_{1,2} 7.55, H-1^{I}), 4.40 (d, 1H, J 12.04, PhCHH), 4.42 (d, 1H, J_{1,2} 8.07, H-1^{II}), 4.45 (d, 1H, J 11.92, PhCHH), 4.48 (d, 1H, J 12.00, PhCHH), 4.50 (d, 1H, J 12.00, PhCHH), 4.52 (d, 1H, J 12.04, PhCHH), 4.54 (d, 1H, J 12.00, PhCHH), 4.57 (d, 1H, J 12.00, PhCHH), 4.64 (d, 1H, J 11.92, PhCHH), 4.99 (dd≈t, 1H, J 8.24, H-2^{II}), 5.08-5.13 (m, 2H, H-3^{I}, H-3^{III}), 5.23 (d, 1H, J_{1,2} 3.31, H-1^{III}), 5.46 (d, 1H, J_{3,4} 2.25, H-4^{II}), 5.54 (d, 1H, J_{3,4} 3.11, H-4^{III}), 7.20-7.40 (m, 20H, ArH); 7.49-7.54 (m, 1H, NHC(O)CF₃). R_{f} 0.4 (PhCH₃-AcOEt, 1:2).

### Preparation of 3-aminopropyl-α-D-galactopyranosyl-(1→3)-β-D-galactopyranosyl-(1→4)-2-acetamido-2-deoxy-β-D-glucopyranoside (5) (Scheme I)

The product 3 (252 mg, 0.198 mmol) was deacetylated according to Zemplen (8h, 40° C.), neutralized with AcOH and concentrated. The TLC (CH₃Cl-MeOH, 10:1) analysis of the obtained product showed two spots: the main spot with R_{f} 0.45,

and another one on the start line (ninhydrin positive spot) that was an indication of partial loss of trifluoroacetyl. Therefore, the product was N-trifluoroacetylated by treatment with CF₃COOMe (0.1 ml) and Et₃N (0.01 ml) in MeOH (10 ml) for 1 h, concentrated and subjected to column chromatography on silica gel (CHCl₃-MeOH, 15:1) to afford the product 4 as a white foam (163 mg, 77%), R_{f} 0.45 (CH₃Cl-MeOH, 10:1). The product 4 was subjected to hydrogenolysis (200 mg Pd/C, 10 ml MeOH, 2 h), filtered, N-defluoroacetylated (5% Et₃N/H₂O, 3 h) and concentrated. Cation-exchange chromatography on Dowex 50λ4-400 (H⁺) (elution with 5% aqueous ammonia) gave the product 5 (90 mg, 98%) as a white foam.

¹H NMR (D₂O, characteristic signals), δ, ppm: 1.94-1.98 (m, 2H, CCH₂C), 2.07 (s, 3H, NHC(O)CH₃), 3.11 (m, J 6.92, 2H, NCH₂), 4.54 and 4.56 (2d, 2H, J_{1,2} 8.06, J_{1,2} 7.87, H-1^{I} and H-1^{II}), 5.16 (d, 1H, J_{1,2} 3.87, H-1^{III}). R_{f} 0.3 (EtOH-BuOH-Py-H₂O-AcOH; 100:10:10:10:3).

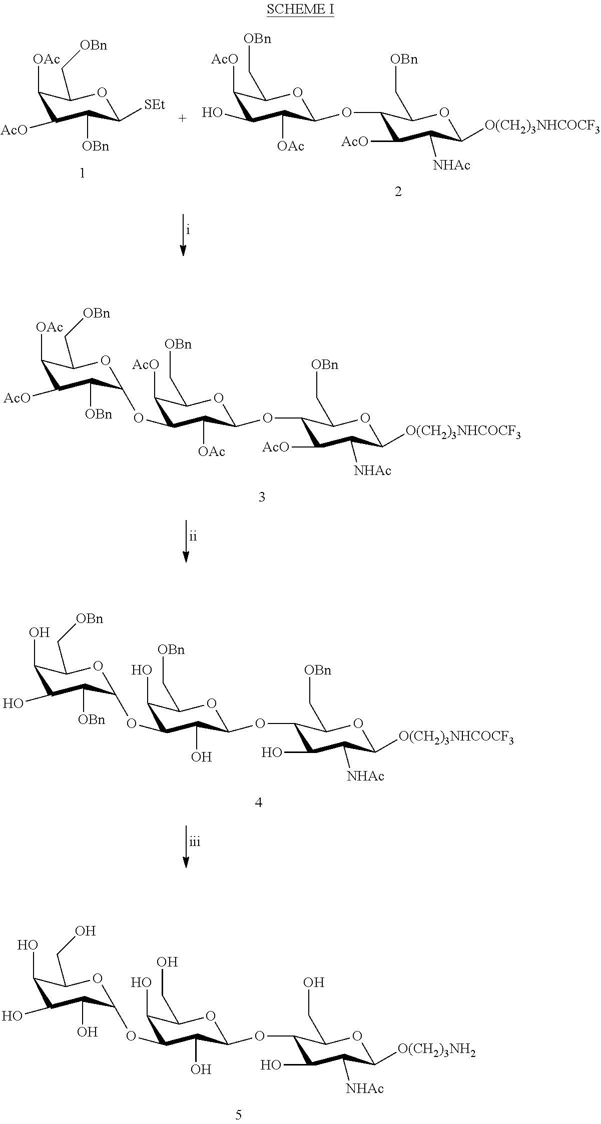

### Preparation of {[2-(2-tert-butoxycarbonylamino-acetylamino)-acetyl]-methoxycarbonylmethyl-amino}-acetic acid methyl ester (8) (Scheme II)

N-Methylmorpholine (11.0 ml, 0.1 mol) was added to a stirred suspension of Boc-glycyl-glycine (23.2 g, 0.1 mol) in 150 ml methylene chloride, the solution was cooled to −15° C. and isobutyl chloroformate (13.64 g, 0.1 mol) was added for 10 min. Then 1-hydroxybenzotriazole and the solution of (methoxycarbonylmethylamino)-acetic acid methyl ester (7) (16.1 g, 0.1 mol) in 50 ml DMF were added to the reaction mixture at the same temperature. The resulting mixture was stirred for 30 min at 0° C. then for 2 h at ambient temperature and evaporated to dryness. The residue was dissolved in 200 ml of methylene chloride and washed with 100 ml 0.5 M HCl and 200 ml 2% aq. NaHCO₃. Solvents were evaporated in vacuum and the residue was purified with column chromatography on silica gel (3% MeOH in CHCl₃) to give pure target compound (34.08 g, 91%) as a colourless glass. TLC: R_{f}=0.40 (5% MeOH in CHCl₃), R_{f}=0.49 (7:1 (v/v) chloroform/methanol).

¹H NMR (500 MHz, [D₆]DMSO, 30° C.) δ, ppm: 7.826 (t, J=5.1 Hz, 1H; NHCO), 6.979 (t, J=5.9 Hz, 1H; NHCOO), 4.348 and 4.095 (s, 2H; NCH₂COO), 3.969 (d, J=5.1 Hz, 2H; COCH₂NH), 3.689 and 3.621 (s, 3H; OCH₃), 3.559 (d, J=5.9 Hz, 2H; COCH₂NHCOO), 1.380 (s, 9H; C(CH₃)₃). R_{f} 0.49 (7:1 (v/v) chloroform/methanol).

### Preparation of {[2-(2-tert-butoxycarbonylamino-acetylamino)-acetyl]-methoxycarbonylmethyl-amino}-acetic acid (9) (Scheme II)

0.2 M aqueous NaOH (325 ml) was added to a stirred solution of {[2-(2-tert-butoxycarbonylamino-acetylamino)-acetyl]-methoxycarbonylmethyl-amino}-acetic acid methyl ester (8) (24.42 g, 65.12 mmol) in methanol (325 ml), reaction mixture was kept for 15 min at ambient temperature, acidified with acetic acid (5 ml) and evaporated to dryness. Column chromatography of the residue on silica gel (methanol-ethyl acetate 1:1) gave the target compound as Na-salt (20.44 g) which was dissolved in methanol/water/pyridine mixture (20:10:1, 350 ml) and passed through ion-exchange column (Dowex 50×4-400, pyridine form, 300 ml) to remove Na cations. Column was washed with the same mixture, eluate evaporated and dried in vacuum to give pure target compound (20.15 g, 86%) as a white solid. TLC: Rf=0.47 (iPrOH/ethyl acetate/water 4:3:1).

¹H NMR (500 MHz, [D₆]DMSO, 30° C.), mixture of cis- and trans-conformers of N-carboxymethylglycine unit c.3:1. Major conformer; δ, ppm: 7.717 (t, J=5 Hz, 1H; NHCO), 7.024 (t, J=5.9 Hz, 1H; NHCOO), 4.051 (s, 2H; NCH₂COOCH₃), 3.928 (d, J=5 Hz, 2H; COCH₂NH), 3.786 (s, 2H; NCH₂COOH), 3.616 (s, 3H; OCH₃), 3.563 (d, J=5.9 Hz, 2H; COCH₂NHCOO), 1.381 (s, 9H; C(CH₃)₃) ppm; minor conformer, δ=7.766 (t, J=5 Hz, 1H; NHCO), 7.015 (t, J=5.9 Hz, 1H; NHCOO), 4.288 (s, 2H; NCH₂COOCH₃), 3.928 (d, J=5 Hz, 2H; COCH₂NH), 3.858 (s, 2H; NCH₂COOH), 3.676 (s, 3H; OCH₃), 3.563 (d, J=5.9 Hz, 2H; COCH₂NHCOO), 1.381 (s, 9H; C(CH₃)₃). R_{f} 0.47 (4:3:1 (v/v/v) i-PrOH/ethyl acetate/water).

### Preparation of {[2-(2-tert-butoxycarbonylamino-acetylamino)-acetyl]-methoxycarbonylmethyl-amino}-acetic acid N-oxysuccinimide ester (Boc-Gly₂(MCM)GlyOSu)(10) (Scheme II)

N,N′-Dicyclohexylcarbodiimide (14.03 g, 68.10 mmol) was added to an ice-cooled stirred solution of {[2-(2-tert-butoxycarbonylamino-acetylamino)-acetyl]-methoxycarbonylmethyl-amino}-acetic acid (26.40 g, 73.13 mmol) and N-hydroxysuccinimide (8.70 g, 75.65 mmol) in DMF (210 ml). The mixture was stirred for 30 min at 0° C. then for 2 h at ambient temperature.

Precipitated N,N′-dicyclohexylurea was filtered off, washed with DMF (80 ml). The filtrate and washings were concentrated and the residue was stirred with Et₂O (500 ml) for 1 h. Ether extract was decanted and the residue was concentrated to give target compound as a white foam (32.57 g, 97%). TLC: R_{f}=0.71 (acetone/acetic acid 40:1).

¹H NMR (500 MHz, DMSO[D₆], 30° C.), mixture of cis- and trans-conformers of N-carboxymethylglycine unit c. 3:2.

Major conformer; δ, ppm: 7.896 (t, J=5.1 Hz, 1H; NHCO), 6.972 (t, J=5.9 Hz, 1H; NHCOO), 4.533 (s, 2H; NCH₂COON), 4.399 (s, 2H; NCH₂COOCH₃), 3.997 (d, J=5.1 Hz, 2H; COCH₂NH), 3.695 (s, 3H; OCH₃), 3.566 (d, J=5.9 Hz, 2H; COCH₂NHCOO), 1.380 (s, 9H; C(CH₃)₃).

Minor conformer; δ, ppm: 7.882 (t, J=5.1 Hz, 1H; NHCO), 6.963 (t, J=5.9 Hz, 1H; NHCOO), 4.924 (s, 2H; NCH₂COON), 4.133 (s, 2H; NCH₂COOCH₃), 4.034 (d, J=5.1 Hz, 2H; COCH₂NH), 3.632 (s, 3H; OCH₃), 3.572 (d, J=5.9 Hz, 2H; COCH₂NHCOO), 1.380 (s, 9H; C(CH₃)₃).

R_{f} 0.71 (40:1 (v/v) acetone/acetic acid).

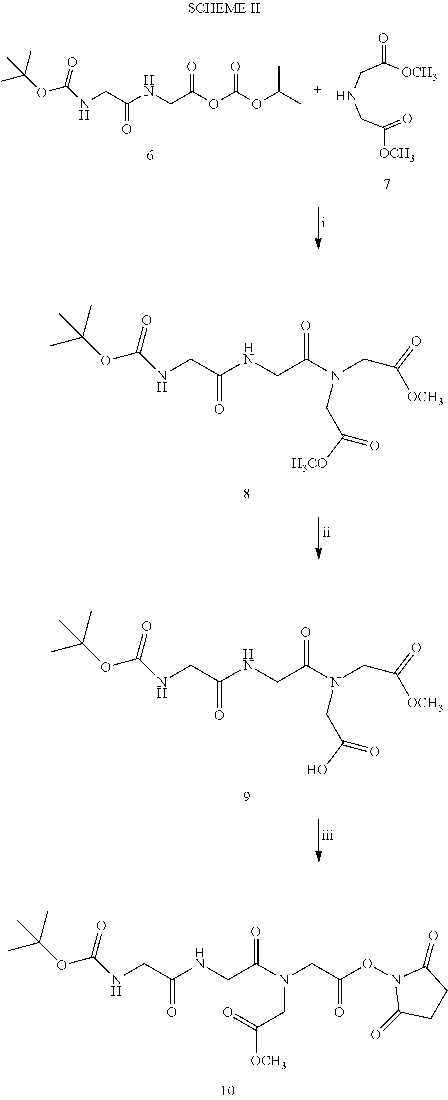

### Preparation of CMG(2) Diamine (16) (Schemes III and IV)

A solution of ethylenediamine (11) (808 mg, 13.47 mmol) and Et₃N (1.87 ml, 13.5 mmol) in DMSO (5 ml) was added to a stirred solution of Boc-Gly₂-(MCM)Gly-OSu (10) (15.42 g, 33.68 mmol) in DMSO (50 ml). The reaction mixture was stirred for 30 min at ambient temperature and acidified with acetic acid (1.2 ml), then fractionated with Sephadex LH-20 column (column volume 1200 ml, eluent-MeOH/water 2:1+0.2% AcOH). Fractions containing compound Boc₂MCMG (12) were combined, solvents evaporated and the residue was concentrated in vacuum. The product was additionally purified by silica gel column chromatography using 2-propanol/ethyl acetate/water (2:6:1) as eluent. Fractions containing pure Boc₂MCMG (12) were combined, solvents evaporated and a residue was dried in vacuum to give target Boc₂MCMG (12) as colourless foam (8.41 g, 84%). TLC: R_{f}=0.48 (ⁱPrOH/ethyl acetate/water 2:3:1).

¹H NMR (500 MHz, [D₆]DMSO, 30° C.), mixture of conformers ˜3:2: 8.166, 8.125, 7.917 and 7.895 (m, total 2H; 2 CONHCH₂), 7.793 (m, 2H; NHCH₂CH₂NH), 7.001 (br. t, 2H; 2 NHCOO), 4.277-3.893 (total 12H; 2 CH₂COO, 4 NCH₂CO), 3.690 and 3.635 (s, total 6H; 2 COOCH₃), 3.567 (d, J=5.8 Hz, 4H; 2CH₂NHCOO), 3.131 (m, 4H; NHCH₂CH₂NH), 1.379 (s, 18H; 2C(CH₃)₃) ppm.

MS, m/z: 769 [M+Na], 785 [M+K].

Trifluoroacetic acid (25 ml) was added to a stirred solution of Boc₂MCMG (12) (4.88 g, 6.535 mmol) in methylene chloride (25 ml) and the solution was kept for 1 h at ambient temperature. Then a reaction mixture was concentrated and the residue was evaporated three times with anhydrous MeOH (50 ml), then a residue was extracted three times with Et₂O (100 ml) to remove traces of trifluoroacetic acid. The resulted precipitate (as a white solid) was dried to give 5.06 g (˜100%) of MCMG (13) as bis-trifluoroacetic salt. TLC: R_{f}=0.23 (ethanol/water/pyridine/acetic acid 5:1:1:1).

¹H NMR (500 MHz, D₂O, 30° C.), mixture of conformers ˜5:4: 4.400-4.098 (total 12H; 2 CH₂COO, 4 NCH₂CO), 3.917 (s, 4H; 2 COCH₂NH₂), 3.829 and 3.781 (s, total 6H; 2 COOCH₃), 3.394 (m, 4H; NHCH₂CH₂NH) ppm.

MS, m/z: 547 [M+H], 569 [M+Na], 585 [M+K].

A solution of Boc-Gly₂-(MCM)Gly-OSu (10) (7.79 g, 16.994 mmol) in DMSO (17 ml) and Et₃N (2.83 ml, 20.4 mmol) was added to the stirred solution of MCMG (13) (5.06 g, 6.796 mmol) in DMSO (13 ml). The reaction mixture after stirring for 2 h at ambient temperature was acidified with acetic acid (4.0 ml) and fractionated with Sephadex LH-20 column chromatography (column volume 1200 ml, eluent-MeOH/water 2:1+0.2% AcOH). Fractions containing pure Boc₂MCMG (14) were combined, solvents evaporated and the residue was dried in vacuum to give target Boc₂MCMG (14) as colourless foam (8.14 g, 97%). TLC: R_{f}=0.25 (ⁱPrOH/ethyl acetate/water 2:3:1).

¹H NMR (500 MHz, [D₆]DMSO, 30° C.), mixture of conformers: 8.393-7.887 (total 6H; 6 CONHCH₂), 7.775 (m, 2H; NHCH₂CH₂NH), 6.996 (br. t, 2H; 2 NHCOO), 4.299-3.730 (total 28H; 4 CH₂COO, 10 NCH₂CO), 3.691 and 3.633 (s, total 12H; 4 COOCH₃), 3.564 (d, J=5.8 Hz, 4H; 2CH₂NHCOO), 3.129 (m, 4H; NHCH₂CH₂NH), 1.380 (s, 18H; 2C(CH₃)₃) ppm.

MS, m/z: 1256 [M+Na], 1271 [M+K].

Boc₂MCMG (14) (606 mg, 0.491 mmol) was dissolved in CF₃COOH (2 ml) and the solution was kept for 30 min at r.t. Trifluoroacetic acid was evaporated in vacuum and the residue was extracted three times with Et₂O (trituration with 25 ml of Et₂O followed by filtration) to remove residual CF₃COOH and the obtained white powder was dried in vacuum. The powder was dissolved in 4 mL of water and then was freeze-dried. Yield of MCMG (15) (TFA salt) was estimated as quantitative (actual weight was larger than theoretical by ˜10% due to stability of hydrates). TLC: R_{f}=0.21 (ethanol/water/pyridine/acetic acid 5:1:1:1).

¹H NMR (500 MHz, [D₂]H₂O, 30° C.), mixture of conformers: 4.430-4.014 (total 28H; 4 CH₂COO, 10 NCH₂CO), 3.911 (s, 4H; 2 COCH₂NH₂), 3.823 and 3.772 (s, total 12H; 4 COOCH₃), 3.386 (m, 4H; NHCH₂CH₂NH) ppm.

MS, m/z: 1034 [M+H], 1056 [M+Na].

To the solution of MCMG (15) (˜0.49 mmol) in water (20 mL) Et₃N (0.5 mL) was added, and the solution was kept for 15 h at r.t. The reaction mixture was evaporated to dryness and the residue was desalted on Sephadex LH-20 column (two methods):

Method A. The residue was dissolved in water (3 ml) and the solution was desalted on Sephadex LH-20 column (column volume 250 mL, eluent-MeOH/water 1:1+0.05 M pyridine acetate). Fractions, containing CMG (16) contaminated with salts were combined separately, evaporated and the residue was desalted again. Combined fractions, containing pure CMG (16), were evaporated to ˜4 ml volume and freeze dried. Yield of CMG (16) (internal salt) was 431 mg (90%).

Method B. The residue was dissolved in water (3 ml) and the solution was desalted on Sephadex LH-20 column (column volume 250 mL, eluent-MeOH/water 1:1+1% conc. aq. NH₃). Fractions, containing pure CMG (16), were evaporated to ˜4 ml volume and freeze dried. The residue (ammonia salt of CMG (16)) was dissolved in ⁱPrOH/water 1:1 mixture (10 mL), Et₃N (0.2 mL) was added, and the solution was evaporated to dryness. This procedure was repeated twice; the residue was dissolved in 4 mL of water and freeze-dried. Yield of the di-Et₃N salt of CMG (16) was 549 mg (95%).

TLC: R_{f}=0.50 (ⁱPrOH/MeOH/acetonitrile/water 4:3:3:4+3% conc. aq. NH₃), or R_{f}=0.43 (ⁱPrOH/EtOH/MeOH/water 1:1:1:1, 0.75M NH₃).

¹H NMR of CMG (16) internal salt (500 MHz, [D₂]H₂O, 30° C.), mixture of conformers: 4.328-4.006 (total 28H; 4 CH₂COO, 10 NCH₂CO), 3.907 (s, 4H; 2 COCH₂NH₂), 3.381 (m, 4H; NHCH₂CH₂NH) ppm.

MS, m/z: 977 [M+H], 999 [M+Na], 1015 [M+K].

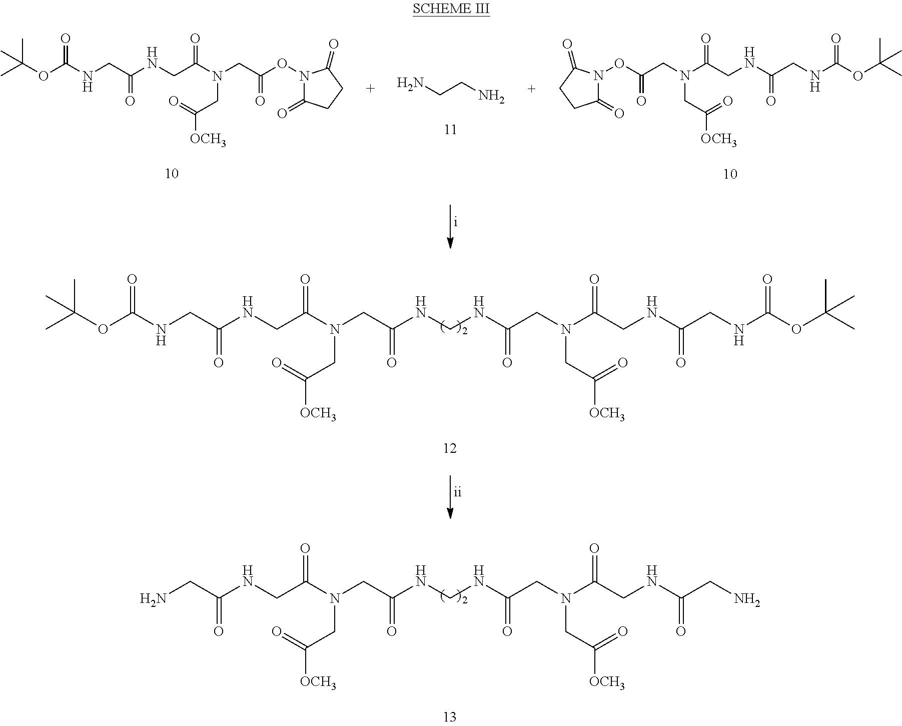

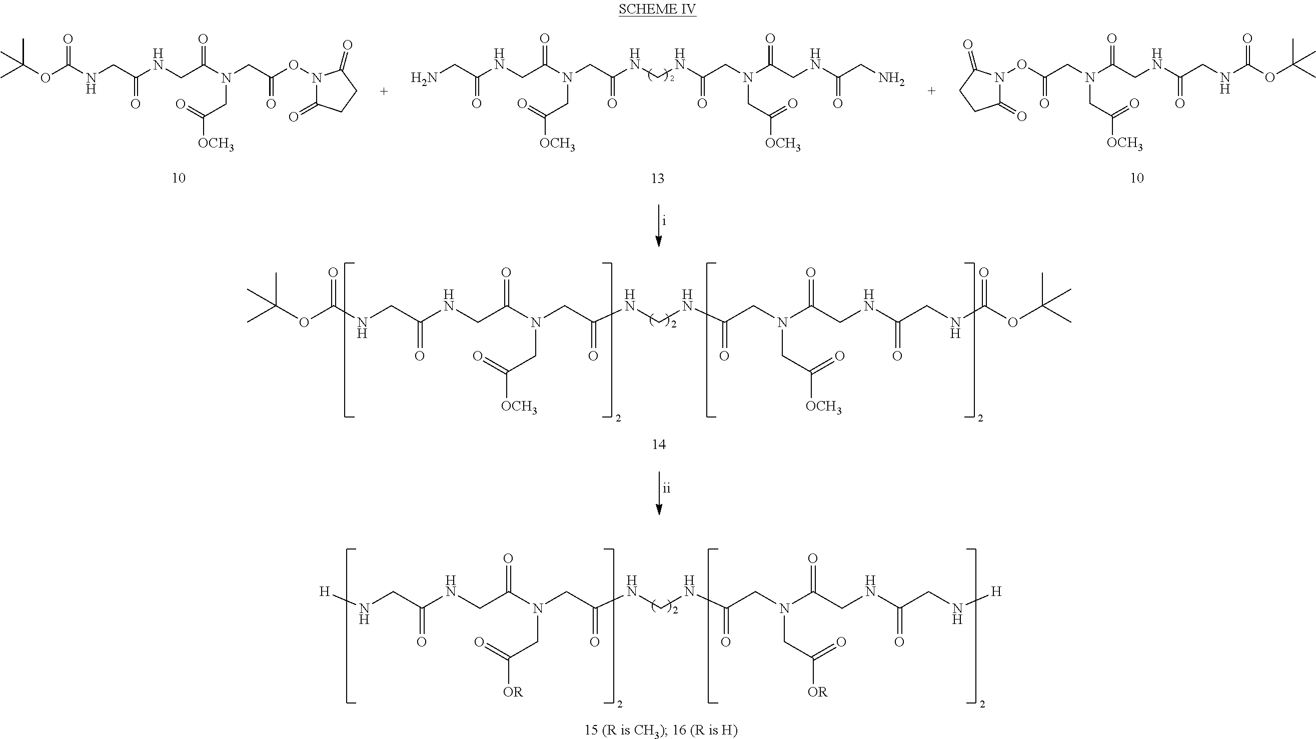

### Preparation of H₂N-CMG(16)-DOPE (20) (Scheme V)

To the intensively stirred solution of CMG (16) (425 mg, 0.435 mmol of internal salt) in i-PrOH/water mixture (i-PrOH/water 3:2, 10 mL) the 1 M aq. solution of NaHCO₃ (0.435 mL, 0.435 mmol) and then the solution of DOPE-Ad-OSu (16) (211 mg, 0.218 mmol) in dichloroethane (0.4 mL) were added. The reaction mixture was stirred for 2 h and then acidified with 0.2 mL of AcOH and evaporated to minimal volume at 35° C. The solid residue was dried in vacuum (solid foam) and then thoroughly extracted with CHCl₃/MeOH mixture (CHCl₃/MeOH 4:1, several times with 10 mL, TLC control). The extracted residue consisted of unreacted CMG(2) and salts (about 50% of CMG (16) was recovered by desalting of combined the residue and a fractions after chromatography on silica gel according to procedure described in the CMG (16) synthesis.). The combined CHCl₃/MeOH extracts (solution of CMG (16)-Ad-DOPE amine, DOPE-Ad-CMG (16)-Ad-DOPE, N-oxysuccinimide and some CMG (16)) were evaporated in vacuum and dried. The obtained mixture was separated on silica gel column (2.8×33 cm, ˜200 mL of silica gel in CHCl₃/MeOH 5:1). The mixture was placed on column in MeOH/CHCl₃/water mixture (MeOH/CHCl₃/water 6:3:1+0.5% of pyridine) and the components were eluted in a stepwise ternary gradient: MeOH/CHCl₃/water composition from 6:3:1 to 6:2:1 and then to 6:2:2 (all with 0.5% of pyridine). DOPE-Ad-CMG(16)-Ad-DOPE was eluted first (R_{f}=0.75, MeOH/CHCl₃/water 3:1:1), followed by desired DOPE-Ad-CMG(16) amine (R_{f}=0.63, MeOH/CHCl₃/water 3:1:1), last eluted was CMG (16) (R_{f}=0.31, MeOH/CHCl₃/water 3:1:1). Fractions, containing pure CMG(16)-Ad-DOPE amine (20) were combined and evaporated to dryness. To remove any low molecular weight impurities and solubilised silica gel the residue was dissolved in ⁱPrOH/water 1:2 mixture (2 mL), and was passed through Sephadex LH-20 column (column volume 130 mL, eluent-ⁱPrOH/water 1:2+0.25% of pyridine). Fractions containing pure CMG(16)-Ad-DOPE amine (20) were combined and evaporated (˜20% of 2-propanol was added to prevent foaming) to dryness, the residue was dissolved in water (˜4 mL) and freeze-dried. Yield of CMG(16)-Ad-DOPE amine (20) was 270 mg (68% on DOPE-Ad-OSu or 34% on CMG(16)).

¹H NMR (500 MHz, [D₂]H₂O/[D₄]CH₃OH 2:1, 30° C.): 5.505 (m, 4H; 2 CH₂CH═CHCH₂), 5.476 (m, 1H; OCH₂CHCH₂O), 4.626 (dd, J_{gem}=11.6 Hz, 1H; OCHCHCH₂O), 4.461-4.084 (total 37H; 4 CH₂COO, 11 NCH₂CO, OCHCHCH₂O, OCH₂CH₂N), 4.002 (s, 2H; COCH₂NH₂), 3.573 (m, 4H; NHCH₂CH₂NH), 2.536-2.463 (m, total 8H; 4 CH₂CO), 2.197 (m, 8H; 2CH₂CH═CHCH₂), 1.807 (m, 8H; 4CH₂CH₂CO), 1.480 (m, 40H; 20 CH₂), 1.063 (˜t, J≈6 Hz, 6H; 2 CH₃) ppm.

MS, m/z: 1831 [M+H].

### Preparation of Galili-CMG(2)-DOPE (22) (Scheme VI)

To a stirred solution of compound 21 (66 mg, 0.079 mmol) in dry DMSO (6 mL) were added 15 μl Et₃N and powdered H₂N-CMG(2)-DOPE (20) (95 mg, 0.0495 mmol) in 3 portions. The mixture was stirred for 24 h at room temperature and then subjected to column chromatography (Sephadex LH-20, i-PrOHH₂O, 1:2, 0.5 v % Py, 0.25 v % AcOH) to yield the crude compound 22 in a form of Py-salt; The compound was lyophilized from water two times, then dissolved again in 10 ml of water, aqueous solution of NaHCO₃ (50 mM) was added to pH 6.5 for obtaining the compound 22 in a form of Na-salt and the solution was subjected to lyophilization. The yield of compound 22 (Na-salt) was 114 mg (86% based on NH₂-CMG₂-DE), R_{f} 0.6 (i-PrOH-MeOH-MeCN-H₂O, 4:3:6:4). ¹H NMR (FIG. 4) (700 MHz, D₂O-CD₃OD, 1:1 (v/v), 40° C.; selected signals) δ, ppm: 1.05 (t, J 7.03 Hz, 6H; 2CH₃), 1.40-1.58 (m, 40H; 20CH₂), 1.73-1.87 (m, 12H; 2×-COCH₂CH₂CH₂CH₂CO and 2×-COCH₂CH₂—), 1.90-1.99 (m, 2H; OCH₂CH₂CH₂N), 2.15-2.25 (m, 11H; 2×-CH₂CH═CHCH₂—, NHC(O)CH₃), 2.39-2.59 (2m, total 12H, 2×-COCH₂CH₂CH₂CH₂CO— and 2×-COCH₂CH₂—) 4.63 (dd, 1H, J 2.51, J 12.20, C(O)OCHHCHOCH₂O—), 4.67 and 4.69 (2d×1H, J_{1,2} 7.81, J_{1,2} 7.95, H-1^{I}, H-1^{II}), 5.30 (d, 1H, J_{1,2}3.88, H-1^{III}), 5.42-5.46 (m, 1H, —OCH₂—CHO—CH₂O—), 5.49-5.59 (m, 4H, 2×-CH═CH—); MALDI TOF mass-spectrum, M/Z: 2567 (M+Na); 2583 (M+K); 2589 (MNa+Na); 2605 (MNa+K); 2611 (MNa₂+Na).

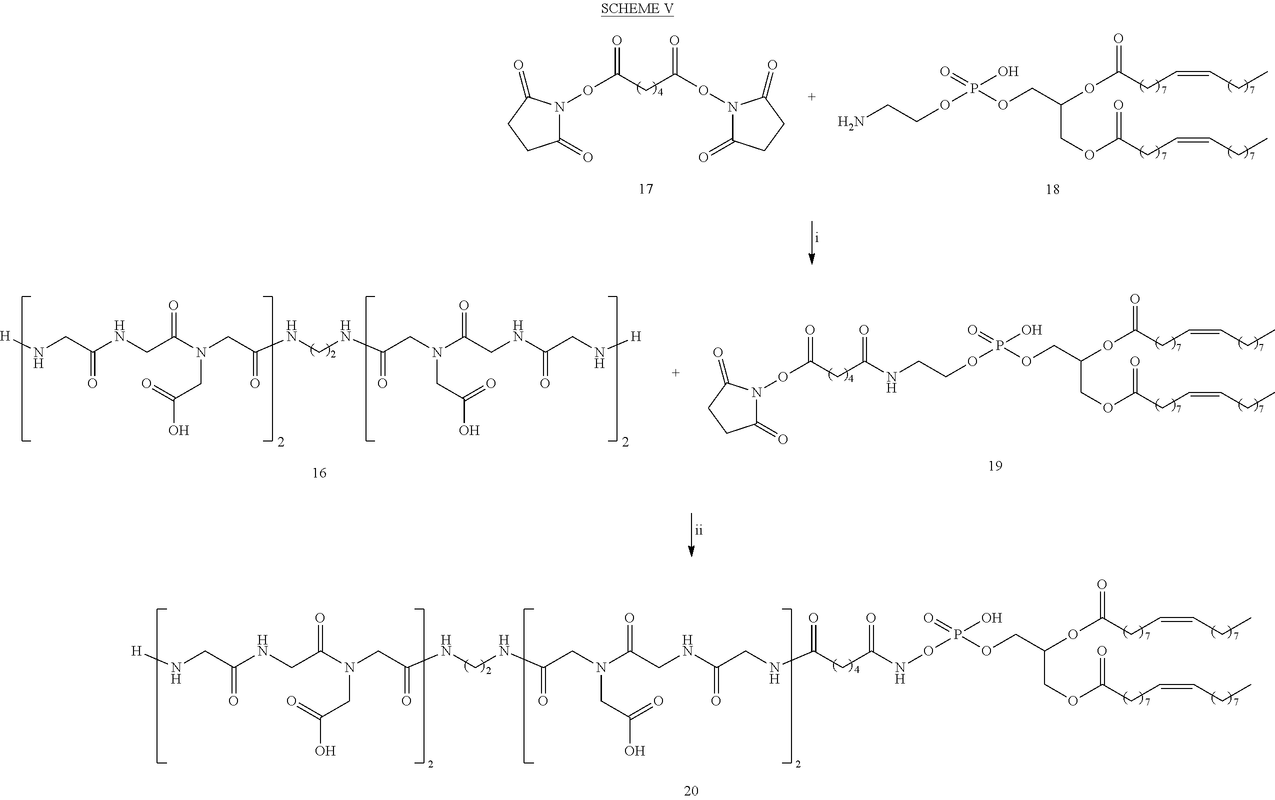

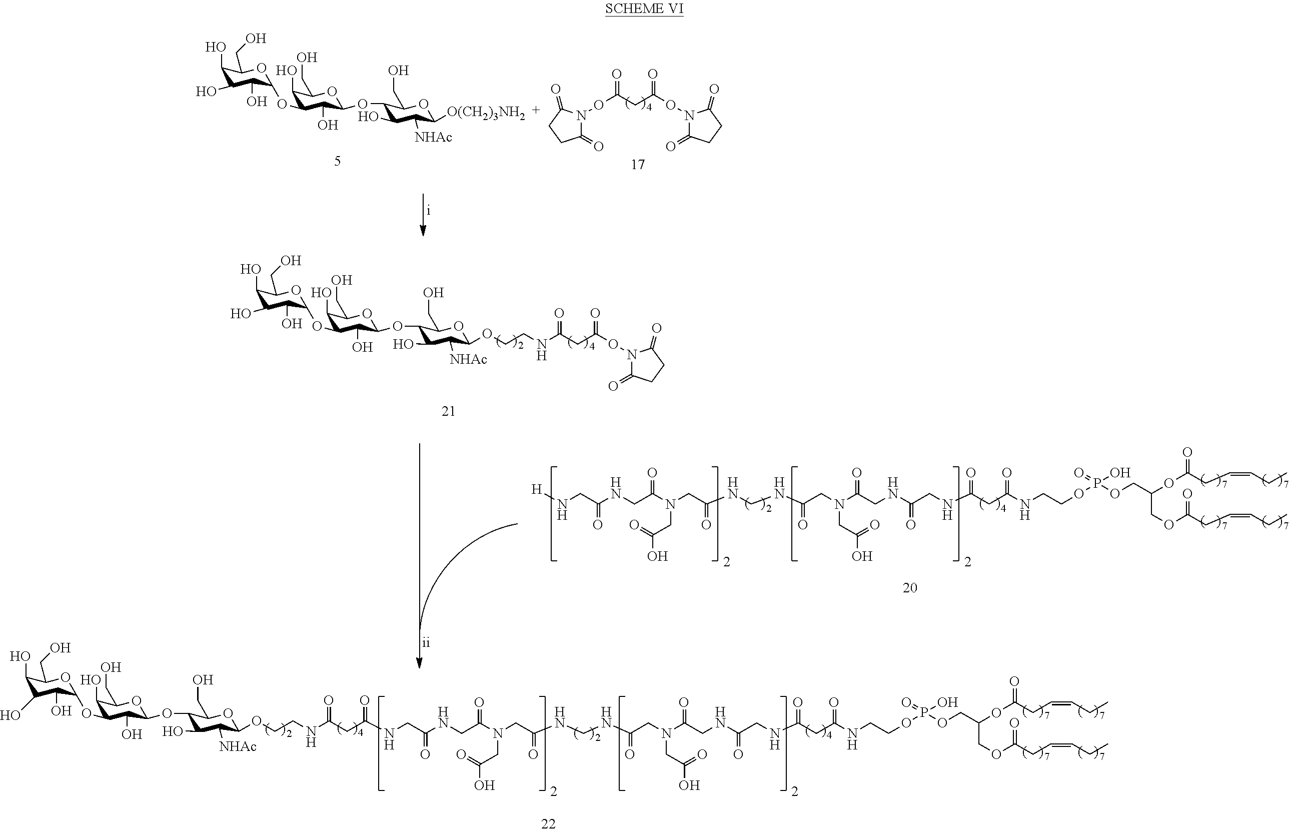

### Example 2: Preparation of the Compound of Formula (II) “Galili-T17 DOPE”

### Preparation of 3-trifluoroacetamidopropyl-3,4-di-O-acetyl-2,6-di-O-benzyl-α-D-galactopyranosyl-(1→3)-2,4-di-O-acetyl-6-O-benzyl-β-D-galactopyranosyl-(1→4)-2-acetamido-3-O-acetyl-6-O-benzyl-2-deoxy-β-D-glucopyranoside (3) (Scheme 1)

The glycosyl acceptor (3-trifluoroacetamidopropyl)-2-acetamido-3-O-acetyl-6-O-benzyl-2-deoxy-4-O-(2,4-di-O-acetyl-6-O-benzyl-β-D-galactopyranosyl)-β-D-glucopyranoside (2) was prepared according to the method disclosed in the publication of Pazynina et al (2008) Russian Journal of Bioorganic Chemistry 34(5), 625-631. A mixture of the glycosyl acceptor 2 (500 mg, 0.59 mmol), thiogalactopyranoside 1 (576 mg, 1.18 mmol), NIS (267 mg, 1.18 mmol), anhydrous CH₂Cl₂ (25 ml) and molecular sieves 4 Å (500 mg) was stirred at −45° C. for 30 min under an atmosphere of Ar. A solution of TfOH (21 μl, 0.236 mmol) in anhydrous CH₂Cl₂ (0.5 ml) was then added. The reaction mixture was stirred for 2 h at −45° C. and the temperature was then increased to −20° C. over 4 h. The mixture was kept at −20° C. overnight. Then extra amounts of thiogalactopyranoside 1 (144 mg, 0.295 mmol), NIS (66 mg, 0.295 mmol) and TfOH (5 μl, 0.06 mmol) were added and the stirring maintained at −20° C. for 2 h before being allowed to slowly warm up to r.t. (1 h). A saturated aqueous solution of Na₂S₂O₃ was then added and the mixture filtered. The filtrate was diluted with CHCl₃ (300 ml), washed with H₂O (2×100 ml), dried by filtration through cotton wool, and concentrated. Gel filtration on LH-20 (CHCl₃-MeOH) afforded the product 3 (600 mg, 80%), as a white foam.

¹H NMR (700 MHz, CDCl₃, characteristic signals), δ, ppm: 1.78-1.82 (m, 4H, CHCHC, OC(O)CH₃), 1.84-1.90 (m, 1H, CHCHC), 1.91, 1.94, 1.97, 1.98, 2.06 (5 s, 5×3H, 4OC(O)CH₃, NH(O)CH₃), 3.23-3.30 (m, 1H, NCHH), 3.59-3.65 (m, 1H, NCHH), 4.05 (m, 1H, H-2^{I}), 4.33 (d, 1H, J_{1,2} 7.55, H-1^{I}), 4.40 (d, 1H, J 12.04, PhCHH), 4.42 (d, 1H, J_{1,2} 8.07, H-1^{II}), 4.45 (d, 1H, J 11.92, PhCHH), 4.48 (d, 1H, J 12.00, PhCHH), 4.50 (d, 1H, J 12.00, PhCHH), 4.52 (d, 1H, J 12.04, PhCHH), 4.54 (d, 1H, J 12.00, PhCHH), 4.57 (d, 1H, J 12.00, PhCHH), 4.64 (d, 1H, J 11.92, PhCHH), 4.99 (dd≈t, 1H, J 8.24, H-2^{II}), 5.08-5.13 (m, 2H, H-3^{I}, H-3^{III}), 5.23 (d, 1H, J_{1,2} 3.31, H-1^{III}), 5.46 (d, 1H, J_{3,4} 2.25, H-4^{II}), 5.54 (d, 1H, J_{3,4} 3.11, H-4^{III}), 7.20-7.40 (m, 20H, ArH); 7.49-7.54 (m, 1H, NHC(O)CF₃). R_{f} 0.4 (PhCH₃-AcOEt, 1:2).

### Preparation of 3-aminopropyl-α-d-galactopyranosyl-(1→3)-β-d-galactopyranosyl-(1→4)-2-acetamido-2-deoxy-β-d-glucopyranoside (5) (Scheme I)

The product 3 (252 mg, 0.198 mmol) was deacetylated according to Zemplen (8h, 40° C.), neutralized with AcOH and concentrated. The TLC (CH₃C1-MeOH, 10:1) analysis of the obtained product showed two spots: the main spot with R_{f} 0.45, and another one on the start line (ninhydrin positive spot) that was an indication of partial loss of trifluoroacetyl. Therefore, the product was N-trifluoroacetylated by treatment with CF₃COOMe (0.1 ml) and Et₃N (0.01 ml) in MeOH (10 ml) for 1 h, concentrated and subjected to column chromatography on silica gel (CHCl₃-MeOH, 15:1) to afford the product 4 as a white foam (163 mg, 77%), R_{f} 0.45 (CH₃Cl-MeOH, 10:1). The product 4 was subjected to hydrogenolysis (200 mg Pd/C, 10 ml MeOH, 2 h), filtered, N-defluoroacetylated (5% Et₃N/H₂O, 3 h) and concentrated. Cation-exchange chromatography on Dowex 50×4-400 (H⁺) (elution with 5% aqueous ammonia) gave the product 5 (90 mg, 98%) as a white foam.

¹H NMR (D₂O, characteristic signals), δ, ppm: 1.94-1.98 (m, 2H, CCH₂C), 2.07 (s, 3H, NHC(O)CH₃), 3.11 (m, J 6.92, 2H, NCH₂), 4.54 and 4.56 (2d, 2H, J_{1,2} 8.06, J_{1,2} 7.87, H-1^{I} and H-1^{II}), 5.16 (d, 1H, J_{1,2} 3.87, H-1^{III}). R_{f} 0.3 (EtOH-BuOH-Py-H₂O-AcOH; 100:10:10:10:3).

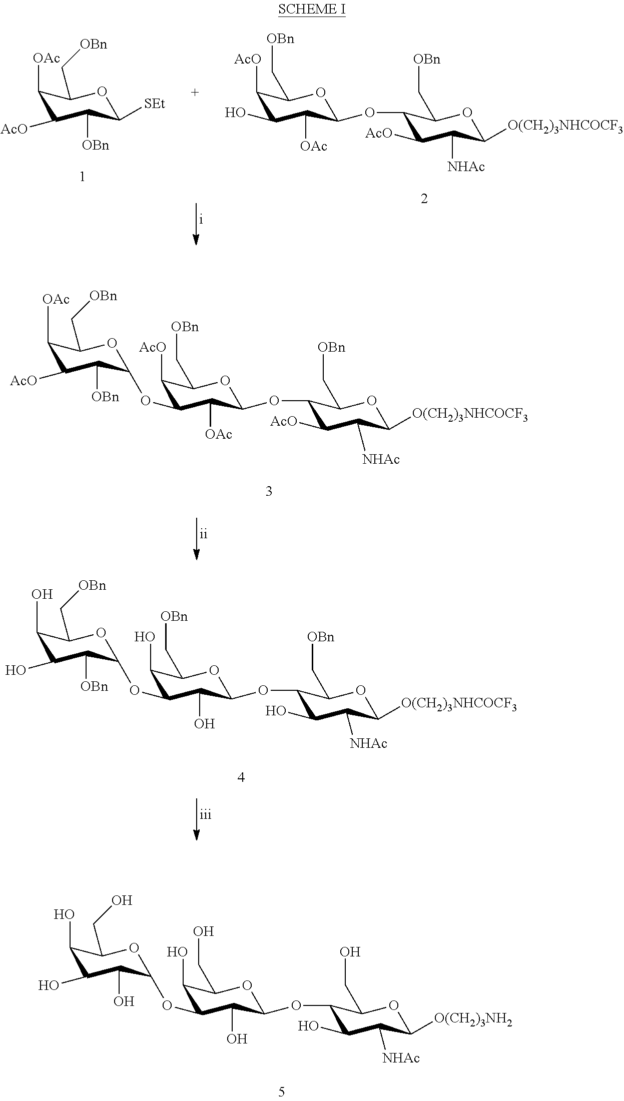

### Preparation of (CF₃COOH.H-Gly₂-NHCH₂)₄C (9) (Scheme II)

Tetraamine (H₂N—CH₂)₄C (7) was synthesized according the method disclosed in the publication of Litherland and Mann (1938) The amino-derivatives of pentaerythritol Part I. Preparation Journal of the Chemical Society, 1588-95. To a stirred solution of the tetraamine 7 (500 mg, 1.52 mmol) in a mixture of 1M aqueous NaHCO₃ (18.2 ml) and i-PrOH (9 ml), Boc-GlyGlyNos (6) (4012 mg, 12.18 mmol) was added (CO₂ evolution, foaming). The reaction mixture was stirred for 30 min, then 6 ml of 1M aqueous NaHCO₃ was added and the mixture stirred overnight. Precipitate of (Boc-Gly₂-HNCH₂)₄C (8) was filtered, washed thoroughly with methanol/water mixture (1:1, 20 ml) and dried in vacuum. Yield 1470 mg (98%), white solid. ¹H NMR (500 MHz, [D₆]DMSO, 30° C.) δ, ppm: 8.491 (t, J=5.6 Hz, 1H; NHCO), 7.784 (t, J=6.6 Hz, 1H; C—CH₂—NHCO), 6.858 (t, J=6 Hz, 1H; NHCOO), 3.696 (d, J=5.6 Hz, 2H; COCH₂NH), 3.675 (d, J=6 Hz, 2H; COCH₂NHCOO), 2.685 (d, J=6.6 Hz, 2H; C—CH₂NH), 1.375 (s, 9H; C(CH₃)₃.

The (Boc-Gly₂-HNCH₂)₄C (8) (1450 mg, 1.466 mmol) was dissolved in CF₃COOH (5 ml) and the solution was kept for 2 h at room temperature. Trifluoroacetic acid was removed under vacuum and the residue was three times extracted with (CH₃CH₂)₂O (slight agitation with 30 ml of (CH₃CH₂)₂O for 30 min., followed by decantation) to eliminate residual CF₃COOH. Solid residue was dried under vacuum, dissolved in a minimum volume of water and passed through a Sephadex LH-20 column and eluted with water. Fractions, containing product 9, were combined, evaporated to c. 5 ml and freeze dried. Yield 1424 mg (93%), white solid. TLC: R_{f} 0.5 (ethanol/conc. NH₃; 2:1 (v/v)).

¹H NMR (500 MHz, [D₂]H₂O, 30° C.) δ, ppm: 4.028 (s, 2H; COCH₂NH), 3.972 (s, 2H; COCH₂NH), 2.960 (s, 2H; C—CH₂NH).

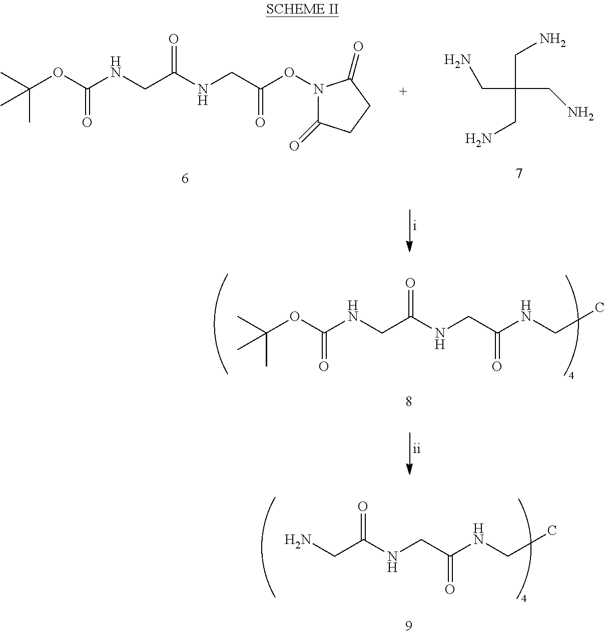

### Preparation of {[2-(2-tert-butoxycarbonylamino-acetylamino)-acetyl]-methoxycarbonylmethyl-amino}-acetic acid methyl ester (11) (Scheme III)

To a stirred solution of (methoxycarbonylmethyl-amino)-acetic acid methyl ester hydrochloride (10) (988 mg, 5 mmol) in DMF (15 ml) were added Boc-GlyGlyNos (6) (3293 mg, 10 mmol) and (CH₃CH₂)₃N (3475 μL, 25 mmol) were added. The mixture was stirred overnight at room temperature and then diluted with o-xylene (70 ml) and evaporated. Flash column chromatography on silica gel (packed in toluene, and eluted with ethyl acetate) resulted in a crude product. The crude product was dissolved in chloroform and washed sequentially with water, 0.5 M NaHCO₃ and saturated KCl. The chloroform extract was evaporated and the product purified on a silica gel column (packed in chloroform and eluted with 15:1 (v/v) chloroform/methanol). Evaporation of the fractions and drying under vacuum of the residue provided a colourless thick syrup of product 11. Yield 1785 mg, (95%). TLC: R_{f}=0.49 (7:1 (v/v) chloroform/methanol).

¹H NMR (500 MHz, [D₆]DMSO, 30° C.) δ, ppm: 7.826 (t, J=5.1 Hz, 1H; NHCO), 6.979 (t, J=5.9 Hz, 1H; NHCOO), 4.348 and 4.095 (s, 2H; NCH₂COO), 3.969 (d, J=5.1 Hz, 2H; COCH₂NH), 3.689 and 3.621 (s, 3H; OCH₃), 3.559 (d, J=5.9 Hz, 2H; COCH₂NHCOO), 1.380 (s, 9H; C(CH₃)₃).

### Preparation of {[2-(2-tert-butoxycarbonylamino-acetylamino)-acetyl]-methoxycarbonylmethyl-amino}-acetic acid (12) (Scheme III)

To a stirred solution of 11 (1760 mg, 4.69 mmol) in methanol (25 ml) 0.2 M aqueous NaOH (23.5 ml) was added and the solution kept for 5 min at room temperature. The solution was then acidified with acetic acid (0.6 ml) and evaporated to dryness. Column chromatography of the residue on silica gel (packed in ethyl acetate and eluted with 2:3:1 (v/v/v) i-PrOH/ethyl acetate/water) resulted in a recovered 11 (63 mg, 3.4%) and target compound 12 (1320 mg). The intermediate product was then dissolved in methanol/water/pyridine mixture (20:10:1, 30 ml) and passed through an ion exchange column (Dowex 50×4-400, pyridine form, 5 ml) to remove residual sodium cations. The column was then washed with the same solvent mixture, the eluent evaporated, the residue dissolved in chloroform/benzene mixture (1:1, 50 ml) and then evaporated and dried under vacuum. Yield of product 12 was 1250 mg (74%), white solid.

TLC: R_{f} 0.47 (4:3:1 (v/v/v) i-PrOH/ethyl acetate/water).

¹H NMR (500 MHz, [D₆]DMSO, 30° C.), mixture of cis- and trans-conformers of N-carboxymethylglycine unit c.3:1. Major conformer; δ, ppm: 7.717 (t, J=5 Hz, 1H; NHCO), 7.024 (t, J=5.9 Hz, 1H; NHCOO), 4.051 (s, 2H; NCH₂COOCH₃), 3.928 (d, J=5 Hz, 2H; COCH₂NH), 3.786 (s, 2H; NCH₂COOH), 3.616 (s, 3H; OCH₃), 3.563 (d, J=5.9 Hz, 2H; COCH₂NHCOO), 1.381 (s, 9H; C(CH₃)₃) ppm; minor conformer, δ=7.766 (t, J=5 Hz, 1H; NHCO), 7.015 (t, J=5.9 Hz, 1H; NHCOO), 4.288 (s, 2H; NCH₂COOCH₃), 3.928 (d, J=5 Hz, 2H; COCH₂NH), 3.858 (s, 2H; NCH₂COOH), 3.676 (s, 3H; OCH₃), 3.563 (d, J=5.9 Hz, 2H; COCH₂NHCOO), 1.381 (s, 9H; C(CH₃)₃).

### Preparation of {[2-(2-tert-Butoxycarbonylamino-acetylamino)-acetyl]-methoxycarbonylmethyl-amino}-acetic acid N-oxysuccinimide ester (Boc-Gly₂(MCMGly)Nos) (13) (Scheme III)

To an ice-cooled stirred solution of 12 (1200 mg, 3.32 mmol) and N-hydroxysuccinimide (420 mg, 3.65 mmol) in DMF (10 ml) was added N,N′-dicyclohexylcarbodiimide (754 mg, 3.65 mmol). The mixture was stirred at 0° C. for 30 min, then for 2 hours at room temperature. The precipitate of N,N′-dicyclohexylurea was filtered off, washed with DMF (5 ml), and filtrates evaporated to a minimal volume. The residue was then agitated with (CH₃CH₂)₂O (50 ml) for 1 hour and an ether extract removed by decantation. The residue was dried under vacuum providing the ester 13 (1400 mg, 92%) as a white foam. TLC: R_{f} 0.71 (40:1 (v/v) acetone/acetic acid).

¹H NMR (500 MHz, [D₆]DMSO, 30° C.), mixture of cis- and trans-conformers of N-carboxymethylglycine unit c. 3:2.

Major conformer; δ, ppm: 7.896 (t, J=5.1 Hz, 1H; NHCO), 6.972 (t, J=5.9 Hz, 1H; NHCOO), 4.533 (s, 2H; NCH₂COON), 4.399 (s, 2H; NCH₂COOCH₃), 3.997 (d, J=5.1 Hz, 2H; COCH₂NH), 3.695 (s, 3H; OCH₃), 3.566 (d, J=5.9 Hz, 2H; COCH₂NHCOO), 1.380 (s, 9H; C(CH₃)₃).

Minor conformer; δ, ppm: 7.882 (t, J=5.1 Hz, 1H; NHCO), 6.963 (t, J=5.9 Hz, 1H; NHCOO), 4.924 (s, 2H; NCH₂COON), 4.133 (s, 2H; NCH₂COOCH₃), 4.034 (d, J=5.1 Hz, 2H; COCH₂NH), 3.632 (s, 3H; OCH₃), 3.572 (d, J=5.9 Hz, 2H; COCH₂NHCOO), 1.380 (s, 9H; C(CH₃)₃).

The ester 11 (1380 mg) was dissolved in DMSO to provide a volume of 6 ml and used as a 0.5 M solution (stored at −18° C.).

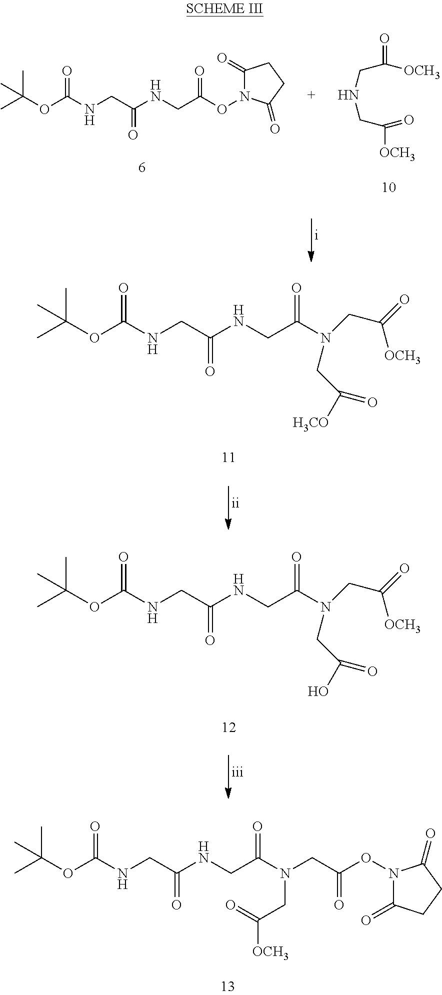 [SMILES: CC(C)(C)OC(=O)NCC(=O)NCC(=O)ON1C(=O)CCC1=O ; COC(=O)CNCC(=O)OC ; COC(=O)CN(CC(=O)OC)C(=O)CNC(=O)CNC(=O)OC(C)(C)C ; COC(=O)CN(CC(=O)O)C(=O)CNC(=O)CNC(=O)OC(C)(C)C ; COC(=O)CN(CC(=O)ON1C(=O)CCC1=O)C(=O)CNC(=O)CNC(=O)OC(C)(C)C]

### Preparation of {CF₃COOH.H-[Gly₂(MCMGly)]Gly₂-NHCH₂}₄C (15) (Scheme IV)

To a stirred solution of (CF₃COOH.H-Gly₂-HNCH₂)₄C (9) (277 mg, 0.265 mmol) in DMSO (2 ml) the ester 11 (1.591 mmol, 3.18 ml of 0.5 M solution in DMSO) and (CH₃CH₂)₃N (295 μL, 2.121 mmol) were added. The mixture was stirred overnight at room temperature, acidified with 150 μL AcOH and solvent removed under vacuum (freeze drying). The residue was extracted three times with (CH₃CH₂)₂O (slight agitation with 20 ml of (CH₃CH₂)₂O for 30 min followed by decantation). The solid residue was dissolved in a minimal volume of acetone and fractionated on silica gel column (packed in acetone and eluted with acetone, 20:2:1 (v/v/v) acetone/methanol/water and 15:2:1 (v/v/v) acetone/methanol/water). Selected fractions were evaporated and the residue was dried under vacuum. The yield of pure {Boc-[Gly₂(MCMGly)]Gly₂-NHCH₂}₄C (14) was 351 mg (68%), white solid. TLC: R_{f} 0.38 (15:2:1 (v/v/v) acetone/methanol/water).

¹H NMR (500 MHz, [D₆]DMSO, 30° C.), mixture of cis- and trans-conformers of N-carboxymethylglycine unit in chain c. 3:2.

Major conformer; δ, ppm: 8.593 (t, J=5 Hz, 1H; NHCO), 8.335 (t, J=5.4 Hz, 1H; NHCO), 7.821 (t, J=6.4 Hz, 1H; C—CH₂—NHCO), 7.786 (t, J=5.1 Hz, 1H; NHCO), 6.993 (t, J=6 Hz, 1H; NHCOO), 4.139 (s, 2H; NCH₂CO), 4.074 (s, 2H; NCH₂COO(CH₃)), 3.985 (d, J=5 Hz, 2H; COCH₂NH), 3.887 (d, J=5.4 Hz, 2H; COCH₂NH), 3.726 (d, J=5.1 Hz, 2H; COCH₂NH), 3.634 (s, 3H; OCH₃), 3.567 (d, J=6 Hz, 2H; COCH₂NHCOO), 2.686 (broad. d, J=6.4 Hz, 2H; C—CH₂NH), 1.379 (s, 9H; C(CH₃)₃).

Minor conformer; δ, ppm: 8.511 (t, J=5 Hz, 1H; NHCO), 8.158 (t, J=5.4 Hz, 1H; NHCO), 7.821 (t, J=6.4 Hz, 1H; C—CH₂—NHCO), 7.786 (t, J=5.1 Hz, 1H; NHCO), 6.993 (t, J=6 Hz, 1H; NHCOO), 4.292 (s, 2H; NCH₂CO), 3.998 (s, 2H; NCH₂COOCH₃), 3.954 (d, J=5 Hz, 2H; COCH₂NH), 3.826 (d, J=5.4 Hz, 2H; COCH₂NH), 3.715 (d, J=5.1 Hz, 2H; COCH₂NH), 3.692 (s, 3H; OCH₃), 3.567 (d, J=6 Hz, 2H; COCH₂NHCOO), 2.686 (broad. d, J=6.4 Hz, 2H; C—CH₂NH), 1.379 (s, 9H; C(CH₃)₃).

The {Boc-[Gly₂(MCMGly)]Gly₂-NHCH₂}₄C (14) (330 mg, 0.168 mmol) was dissolved in CF₃COOH (2 ml) and the solution was kept for 40 min at room temperature.

Trifluoroacetic acid was evaporated under vacuum, the residue extracted three times with (CH₃CH₂)₂O (slight agitation with 20 ml of (CH₃CH₂)₂O for 30 min followed by decantation) to eliminate residual CF₃COOH, and then dried under vacuum. The yield of {CF₃COOH.H-[Gly₂(MCMGly)]Gly₂-NHCH₂}₄C (15) was 337 mg (99%), white solid.

¹H NMR (500 MHz, [D₂]H₂O, 30° C.), mixture of cis- and trans-conformers of N-carboxymethylglycine unit in chain c. 11:10.

Major conformer; δ, ppm: 4.370 (s, 2H; NCH₂CO), 4.265 (s, 2H; NCH₂COOCH₃), 4.215 (s, 2H; COCH₂NH), 4.138 (s, 2H; COCH₂NH), 3.968 (s, 2H; COCH₂NH), 3.919 (s, 2H; COCH₂NH₂⁺), 3.775 (s, 3H; OCH₃), 2.914 (s, 2H; C—CH₂NH).

Minor conformer; δ, ppm: 4.431 (s, 2H; NCH₂CO), 4.241 (s, 2H; NCH₂COOCH₃), 4.239 (s, 2H; COCH₂NH), 4.074 (s, 2H; COCH₂NH), 3.960 (s, 2H; COCH₂NH), 3.919 (s, 2H; COCH₂NH₂⁺), 3.829 (s, 3H; OCH₃), 2.914 (s, 2H; C—CH₂NH).

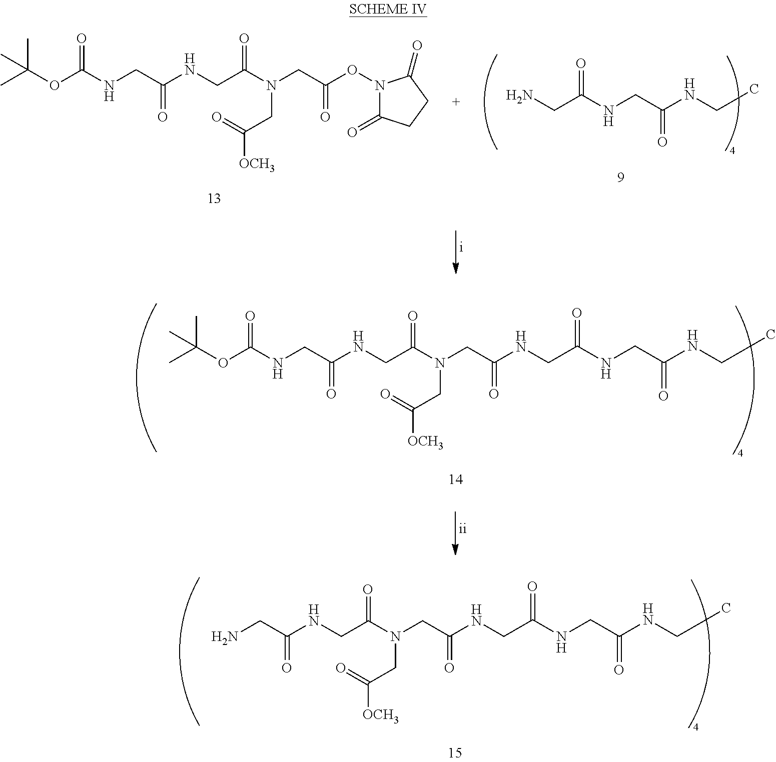

### Preparation of {CF₃COOH.H-[Gly₂(MCMGly)]₂Gly₂-NHCH₂}₄C (Scheme V)

To a stirred solution of (CF₃COOH.H-[Gly₂(MCMGly)]Gly₂-HNCH₂)₄C (15) (272 mg, 0.135 mmol) in DMSO (2 ml) the ester (13) (0.809 mmol, 1.62 ml of 0.5 M solution in DMSO) and (CH₃CH₂)₃N (112 μL, 0.809 mmol) were added. The mixture was stirred overnight at room temperature, acidified with 70 μL AcOH and solvent removed under vacuum (freeze drying). The residue was extracted three times with (CH₃CH₂)₂O (slight agitation with 15 ml of (CH₃CH₂)₂O for 30 min followed by decantation). Solid residue was dissolved in a minimal volume of 7:1 (v/v) acetone/methanol mixture and fractionated on a silica gel column (packed in acetone and eluted with 7:1 (v/v) acetone/methanol, 10:2:1 (v/v/v), 9:2:1 (v/v/v), 8:2:1 (v/v/v) acetone/methanol/water). Selected fractions were evaporated and the residue was dried in vacuum. The yield of pure {Boc-[Gly₂(MCMGly)]₂Gly₂-NHCH₂}₄C (16) was 279 mg (71%), white solid. TLC: R_{f} 0.42 (8:2:1 (v/v/v) acetone/methanol/water).

¹H NMR (500 MHz, [D₆]DMSO, 30° C.), mixture of conformers by two N-carboxymethyl-glycine units per chain, δ, ppm: 8.604, 8.519, 8.397, 8.388, 8.346, 8.211, 8.200, 8.167, 8.034, 8.024, 7.925, 7.912, 7.819 and 7.773 (t, 6H; 6NHCO), 6.992 (t, J=5.9 Hz, 1H; NHCOO), 4.302-3.723 (18H; 2 NCH₂CO, 2 NCH₂COOCH₃, 5 COCH₂NH), 3.692, 3.689 and 3.632 (s, 6H; 2 OCH₃), 3.566 (d, J=5.9 Hz, 2H; COCH₂NHCOO), 2.686 (broad. d, 2H; C—CH₂NH), 1.380 (s, 9H; C(CH₃)₃).

The {Boc-[Gly₂(MCMGly)]₂Gly₂-NHCH₂}₄C (16) (269 mg, 91.65 μmol) was dissolved in CF₃COOH (2 ml) and the solution was kept for 40 min at room temperature. Trifluoroacetic acid was evaporated under vacuum, the residue extracted three times with (CH₃CH₂)₂O (slight agitation with 15 ml of (CH₃CH₂)₂O for 30 min followed by decantation) to remove residual CF₃COOH, and then dried under vacuum. The yield of {CF₃COOH.H-[Gly₂(MCMGly)]₂Gly₂-NHCH₂}₄C was 270 mg (98%), white solid.

¹H NMR (500 MHz, [D₂]H₂O, 30° C.), mixture of conformers by two N-carboxymethyl-glycine units per chain, δ, ppm: 4.441-3.963 (singlets, 18H; 2 NCH₂CO, 2 NCH₂COOCH₃, 5 COCH₂NH), 3.920 (s, 2H; COCH₂NH₂⁺), 3.833, 3.824, 3.780 and 3.773 (s, 6H; 2 OCH₃), 2.918 (s, 2H; C—CH₂NH).

### Preparation of {CF₃COOH.H-[Gly₂(MCMGly)]₃Gly₂-NHCH₂}₄C (Scheme V)

To a stirred solution of (CF₃COOH.H-[Gly₂(MCMGly)]₂Gly₂-HNCH₂)₄C (175 mg, 58.5 μmol) in DMSO (2 ml) the ester 13 (0.351 mmol, 0.702 ml of 0.5 M solution in DMSO) and (CH₃CH₂)₃N (49 μL, 0.351 mmol) were added. The mixture was stirred overnight at room temperature, acidified with 30 μL AcOH and solvent removed under vacuum (freeze drying). The residue was dissolved in a minimal volume of a mixture of 1:1 (v/v) acetonitrile/water and fractionated on a Sephadex LH-20 column (eluted with 1:1 (v/v) acetonitrile/water). Selected fractions were evaporated and the residue was dried in vacuum. The yield of pure {Boc-[Gly₂(MCMGly)]₃Gly₂-NHCH₂}₄C was 279 mg (71%), white solid. TLC: R_{f} 0.42 (8:2:1 (v/v/v) acetone/methanol/water). Fractions containing {Boc-[Gly₂(MCMGly)]₃Gly₂-NHCH₂}₄C were combined, evaporated to c. 2 ml volume and freeze dried. The initial yield was 215 mg (94%). Additional purification on a silica gel column (packed in acetonitrile and eluted with 4:5:2 (v/v/v) i-PrOH/acetonitrile/water) resulted in 169 mg of {Boc-[Gly₂(MCMGly)]₃Gly₂-NHCH₂}₄C (yield 74%, white solid). TLC: R_{f} 0.45 (4:5:2 (v/v/v) i-PrOH/acetonitrile/water).

¹H NMR (500 MHz, [D₆]DMSO, 30° C.), mixture of conformers by three N-carboxymethyl-glycine units per chain, δ, ppm: 8.594-7.772 (triplets, together 8H; 8NHCO), 6.989 (t, J=5.6 Hz, 1H; NHCOO), 4.303-3.722 (26H; 3 NCH₂CO, 3 NCH₂COOCH₃, 7 COCH₂NH), 3.692 and 3.632 (s, 9H; 3 OCH₃), 3.565 (d, J=5.6 Hz, 2H; COCH₂NHCOO), 2.687 (broad. d, 2H; C—CH₂NH), 1.380 (s, 9H; C (CH₃)₃).

The {Boc-[Gly₂(MCMGly)]₃Gly₂-NHCH₂}₄C (146 mg, 37.36 μmol) was dissolved in CF₃COOH (1 ml) and the solution was kept for 40 min at room temperature. Trifluoroacetic acid was evaporated under vacuum, the residue extracted three times with (CH₃CH₂)₂O (slight agitation with 10 ml of (CH₃CH₂)₂O for 30 min followed by decantation) to remove residual CF₃COOH, and then dried under vacuum. The yield of {CF₃COOH.H-[Gly₂(MCMGly)]₃Gly₂-NHCH₂}₄C was 147 mg (99%), white solid.

¹H NMR (500 MHz, [D₂]H₂O, 30° C.), mixture of conformers by three N-carboxymethyl-glycine units per chain, δ, ppm: 4.446-3.964 (singlets, 26H; 3 NCH₂CO, 3 NCH₂COOCH₃, 7 COCH₂NH), 3.924 (s, 2H; COCH₂NH₂⁺), 3.836, 3.828, 3.824, 3.783, 3.778 and 3.773 (s, 9H; 3 OCH₃), 2.919 (s, 2H; C—CH₂NH).

### Preparation of {CF₃COOH.H-[Gly₂(MCMGly)]₄Gly₂-NHCH₂}₄C (Scheme V)

To a stirred solution of (CF₃COOH.H-Gly₂(MCMGly)]₃-HNCH₂)₄C (68 mg, 17.16 μmol) in DMSO (1 ml) the ester 13 (0.137 mmol, 0.275 ml of 0.5 M solution in DMSO) and (CH₃CH₂)₃N (14.3 μL, 0.103 mmol) were added. The mixture was stirred overnight at room temperature, acidified with 100 μL AcOH and solvent removed under vacuum (freeze drying). The residue was dissolved in a minimal volume of a mixture of 1:1 (v/v) acetonitrile/water (0.25% AcOH) and fractionated on a Sephadex LH-20 column (eluted with 1:1 (v/v) acetonitrile/water (0.25% AcOH)). Fractions containing {Boc-[Gly₂(MCMGly)]₄Gly₂-NHCH₂}₄C were combined, evaporated to c. 2 ml volume and freeze dried. The yield was 81 mg (96%), white solid. TLC: R_{f} 0.24 (4:5:2 (v/v/v) i-PrOH/acetonitrile/water).

¹H NMR (500 MHz, [D₆]DMSO, 30° C.), mixture of conformers by four N-carboxymethyl-glycine units per chain, δ, ppm: 8.590-7.773 (triplets, 10H; 10NHCO), 6.989 (t, J=5.6 Hz, 1H; NHCOO), 4.303-3.722 (34H; 4 NCH₂CO, 4 NCH₂COOCH₃, 9 COCH₂NH), 3.691 and 3.631 (5, 12H; 4 OCH₃), 3.565 (d, J=5.6 Hz, 2H; COCH₂NHCOO), 2.684 (broad. d, 2H; C—CH₂NH), 1.379 (s, 9H; C(CH₃)₃).

The {Boc-[Gly₂(MCMGly)]₄Gly₂-NHCH₂}₄C (74 mg, 15.16 μmol) was dissolved in CF₃COOH (1 ml) and the solution was kept for 40 min at room temperature. Trifluoroacetic acid was evaporated under vacuum, the residue extracted three times with (CH₃CH₂)₂O (slight agitation with 10 ml of (CH₃CH₂)₂O for 30 min followed by decantation) to remove residual CF₃COOH, and then dried under vacuum. The yield of {CF₃COOH.H-[Gly₂(MCMGly)]₄Gly₂-NHCH₂}₄C was 72 mg (96%), white solid.

¹H NMR (500 MHz, [D₂]H₂O, 30° C.), mixture of conformers by four N-carboxymethyl-glycine units per chain, δ, ppm: 4.446-3.964 (singlets, 34H; 4 NCH₂CO, 4 NCH₂COOCH₃, 9 COCH₂NH), 3.925 (s, 2H; COCH₂NH₂⁺), 3.836, 3.829, 3.827, 3.822, 3.783, 3.779, 3.777 and 3.772 (s, 12H; 4 OCH₃), 2.919 (s, 2H; C—CH₂NH).

### Preparation of {CF₃COOH.H-[Gly₂(MCMGly)]₅Gly₂-NHCH₂}₄C (23) (Scheme V)

To a stirred solution of (CF₃COOH.H-Gly₂(MCMGly)]₄-HNCH₂)₄C (16.8 mg, 3.403 μmol) in DMSO (1 ml) the ester 13 (27.2 μmol, 63 μl of 0.5 M solution in DMSO) and (CH₃CH₂)₃N (3 μl, 21.6 μmol) were added. The mixture was stirred overnight at room temperature, acidified with 100 μL AcOH and solvent removed under vacuum (freeze drying). The residue was dissolved in a minimal volume of a mixture of 1:1 (v/v) acetonitrile/water (0.25% AcOH) and fractionated on a Sephadex LH-20 column (eluted with 1:1 (v/v) acetonitrile/water (0.25% AcOH)). Fractions containing {Boc-[Gly₂(MCMGly)]₅Gly₂-NHCH₂}₄C (22) were combined, evaporated to c. 1 ml volume and freeze dried. The yield was 19 mg (95%), white solid. TLC: R_{f} 0.15 (4:3:2 (v/v/v) PrOH/acetonitrile/water).

¹H NMR (500 MHz, [D₆]DMSO, 30° C.), mixture of conformers by five N-carboxymethyl-glycine units per chain, δ, ppm: 8.595-7.772 (triplets, 12H; 12NHCO), 6.989 (t, J=5.6 Hz, 1H; NHCOO), 4.303-3.723 (42H; 5 NCH₂CO, 5 NCH₂COOCH₃, 11 COCH₂NH), 3.692 and 3.631 (s, 15H; 5 OCH₃), 3.565 (d, J=5.6 Hz, 2H; COCH₂NHCOO), 2.686 (broad. d, 2H; C—CH₂NH), 1.380 (s, 9H; C(CH₃)₃).

The {Boc-[Gly₂(MCMGly)]₅Gly₂-NHCH₂}₄C (22) (19 mg, 3.25 μmol) was dissolved in CF₃COOH (0.5 ml) and the solution was kept for 40 min at room temperature. Trifluoroacetic acid was evaporated under vacuum, the residue extracted three times with (CH₃CH₂)₂O (slight agitation with 5 ml of (CH₃CH₂)₂O for 30 min followed by decantation) to remove residual CF₃COOH, and then dried under vacuum. Yield of {CF₃COOH.H-[Gly₂(MCMGly)]₅Gly₂-NHCH₂}₄C (23) was 20 mg (99%), white solid.

¹H NMR (500 MHz, [D₂]H₂O, 30° C.), mixture of conformers by five N-carboxymethyl-glycine units per chain, δ, ppm: 4.446-3.965 (singlets, 42H; 5 NCH₂CO, 5 NCH̅₂COOCH₃, 11 COCH₂NH), 3.924 (s, 2H; COCH₂NH₂⁺), 3.835, 3.829, 3.827, 3.825, 3.823, 3.783, 3.779, 3.777 and 3.773 (s, 15H; 5 OCH₃), 2.919 (s, 2H; C—CH₂NH).

### Preparation of [CF₃COOH.H-(Gly₂CMGly)₅Gly₂-NHCH₂]₄C, Et₃N-salt (24) (Scheme V)

To a solution of product 23 (463 mg, 0.07835 mmol) in water (26 mL), Et₃N (523 μL, 3.761 mmol) was added and the solution kept for 18 h at r.t. After evaporation the residue was freeze-dried in vacuum. Yield of product 24 was 587 mg (98%), white solid. TLC: R_{f} 0.39 (1:2:1 (v/v/v) CHCl₃/MeOH/water).

¹H NMR (600 MHz, [D₂]H₂O, 30° C.) δ, ppm: 4.309-3.919 (176H; 20 NCH₂CO, 20 NCH₂COOH, 48 COCH₂NH), 3.226 (q, 120H, J=7.3 Hz; 60 NCH₂CH₃), 2.964 (broad. s, 8H; 4C—CH₂NH), 1.305 (t, 180H, J=7.3 Hz; 60 NCH₂CH₃).

MALDI TOF mass-spectrum, M/Z: 5174, M+H; 5196, M+Na.

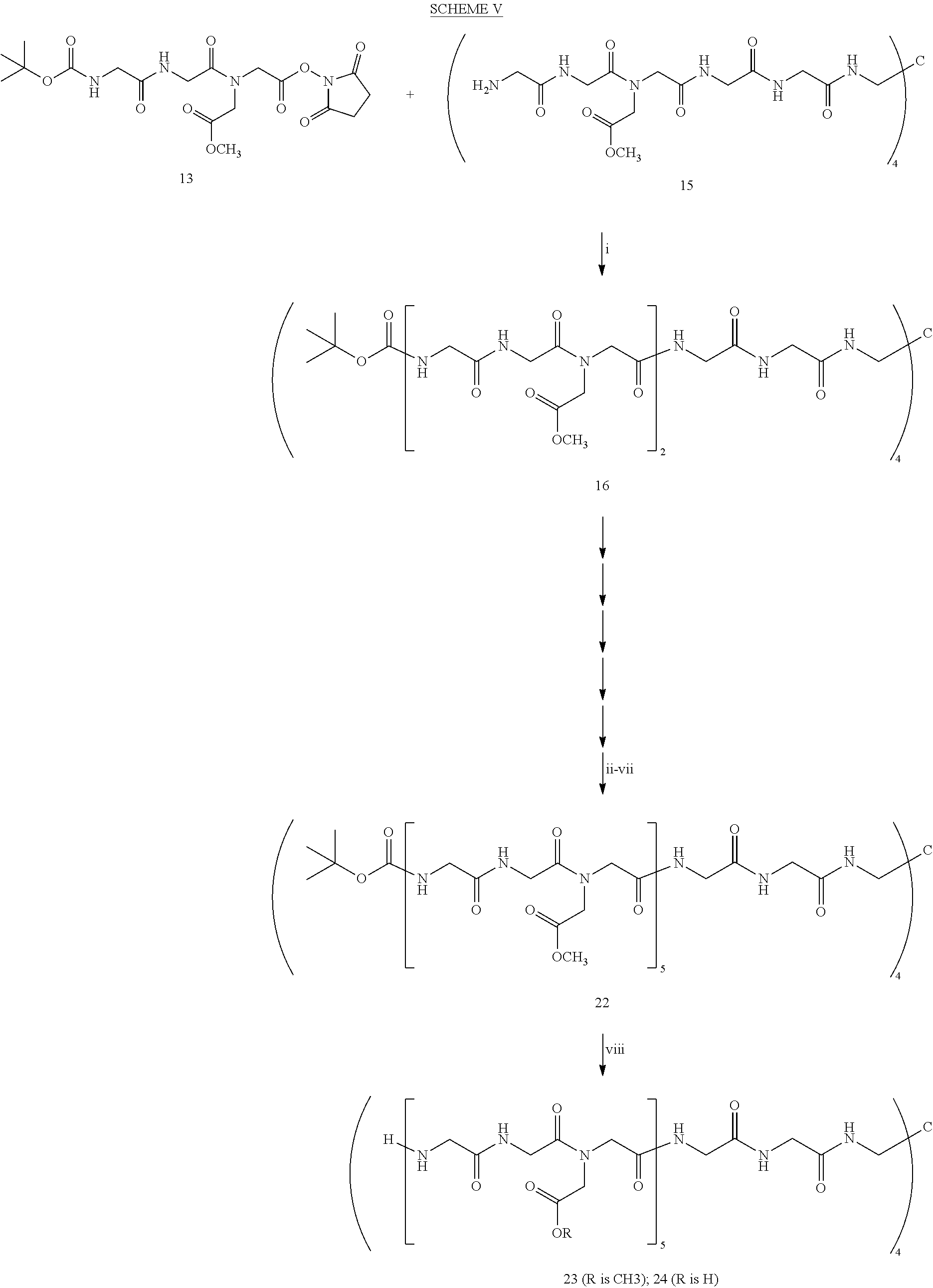

### Preparation of Activated 1,2-O-dioleoyl-sn-glycero-3-phosphatidylethanolamine (DE-Ad-OSu)(27) (Scheme VI)

To a solution of bis(N-hydroxysuccinimidyl) adipate (25) (70 mg, 205 μmol) in dry N,N-dimethylformamide (1.5 ml), 1,2-O-dioleoyl-sn-glycero-3-phosphatidylethanolamine (7) (40 μmol) in chloroform (1.5 ml) was added, followed by triethylamine (7 μl). The mixture was kept for 2 h at room temperature, then neutralized with acetic acid and partially concentrated under vacuum. Column chromatography (Sephadex LH-20, 1:1 chloroform-methanol, 0.2% acetic acid) of the residue yielded the product 27 (37 mg, 95%) as a colorless syrup.

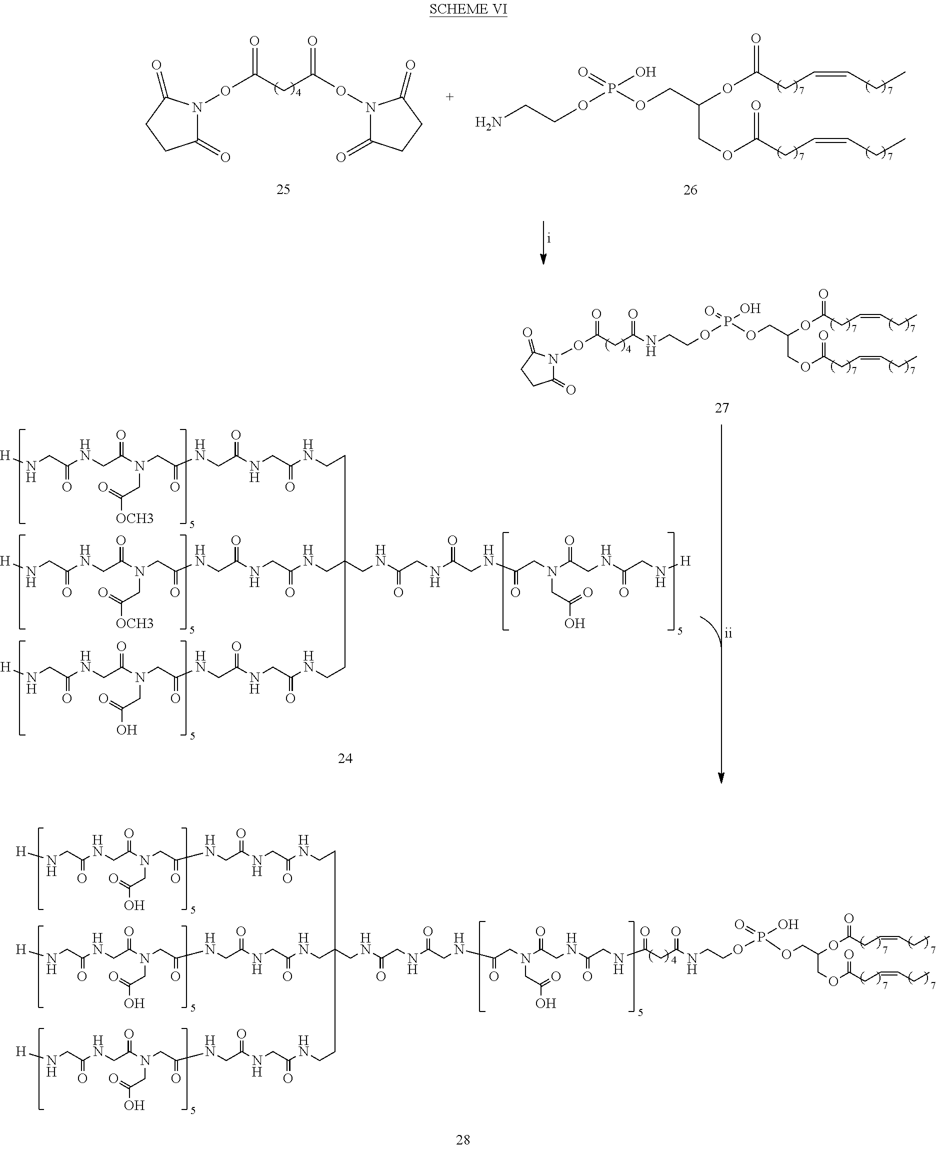

¹H NMR (CDCl₃/CD₃OD, 2:1) 5.5 (m, 4H, 2×(—CH═CH—), 5.39 (m, 1H, —OCH₂—CHO—CH₂O—), 4.58 (dd, 1H, J=3.67, J=11.98, —CCOOHCH—CHO—CH₂O—), 4.34 (dd, 1H, J=6.61, J=11.98, —CCOOHCH—CHO—CH₂O—), 4.26 (m, 2H, PO—CH₂—CH₂—NH₂), 4.18 (m, 2H, —CH₂—OP), 3.62 (m, 2H, PO—CH₂—CH₂—NH₂), 3.00 (s, 4H, ONSuc), 2.8 (m, 2H, —CH₂—CO (Ad), 2.50 (m, 4H, 2×(—CH₂—CO), 2.42 (m, 2H, —CH₂—CO (Ad), 2.17 (m, 8H, 2×(—CH₂—CH₂—CH₂—), 1.93 (m, 4H, COCH₂CH₂CH₂CH₂CO), 1.78 (m, 4H, 2×(COCH₂CH₂—), 1,43, 1.47 (2 bs, 40H, 20 CH₂), 1.04 (m, 6H, 2 CH₃). R_{f} 0.5 (chloroform-methanol-water, 6:3:0.5.

### Preparation of [H-(Gly₂CMGly)₅Gly₂-NHCH₂]₃[DE-CO(CH₂)₄CO-(Gly₂CMGly)₅Gly₂-NHCH₂]C, Na, Et₃N-Salt (28) (Scheme VI)

To a stirred solution of product 24 (522 mg, 0.06821 mmol) in water/2-propanol mixture (16 mL, 2:3) 1M NaHCO₃ (547 μL, 0.547 mmol) and a solution of DE-Ad-OSu (27) (66.1 mg, 0.06821 mmol) in dichloroethane (368 μL) were added, and the solution was stirred for 1.5 h at r.t. After acidification with AcOH (94 μL) the solution was evaporated and the residue was dried in vacuum. Dried mixture was dissolved in 3 mL of water/MeOH (15:1) and put on a C18 reverse phase column (˜45 mL of phase washed with 75% MeOH and then with water/MeOH 15:1). Substances were eluted sequentially with water/MeOH (15:1-50 mL; 9:1-50 mL; 7.5:2.5-50 mL; 1:1-50 mL; 2.5:7.5-100 mL). Unreacted 24 was eluted with water/MeOH 15:1 (Na salt by NMR data, 116 mg, 30.8% of recovery) and with water/MeOH 9:1 (Et₃N salt by NMR data, 63 mg, 13.6% of recovery). Target (H-CMG₅)₃C(CMG₅-Ad-DE) (28) was eluted with water/MeOH 1:1. Yield of pure freeze-dried product 28 was 135 mg (25.5% on (24)), white solid. TLC (1:2:1 (v/v/v) MeOH/ethyl acetate/water): 24 R_{f} 0.06; 28 R_{f} 0.17.

(H-CMG₅)₃C(CMG₅-Ad-DE) Na₁(Et₃N)₂₀ (28): ¹H NMR (700 MHz, [D₂]H₂O/[D₄]CH₃OH 2:1 (v/v), 30° C.) δ, ppm: 5.561 (m, 4H; 2 cis CH═CH of DE), 5.454 (m, 1H; OCH₂—CH(OCO)CH₂O of DE), 4.629 (dd, 1H, J=12.3 Hz/2 Hz; OCH₂—CH(OCO)CHOCO of DE), 4.462-4.057 (181H; 20 NCH₂CO, 20 NCH₂COOH, 48 COCH₂NH, OCH₂—CH(OCO)CHOCO of DE, OCH₂CH₂NH of DE), 3.597 (t, 2H, J=5 Hz; OCH₂CH₂NH of DE), 3.226 (q, 102H, J=7.3 Hz; 51 NCH₂CH₃), 3.099 (broad. s, 8H; 4C—CH₂NH), 2.557, 2.532, 2.522 and 2.456 (triplets, total 8H; 4 CO—CH₂CH₂), 2.203 (˜dd, 8H, J=12 Hz/5.8 Hz; 2CH₂—CH═CH—CH₂ of DE), 1.807 and 1.783 (multiplets, 8H; 4 CO—CH₂CH₂), 1.526 and 1.475 (overlapping m and t, total 193H; m, 20 CH₂ of DE; t, J=7.3 Hz, 51 NCH₂CH₃), 1.063 (t, 6H, J=7 Hz; 2 CH₃ of DE).

MALDI TOF mass-spectrum, M/Z: 6028, M+H; 6050, M+Na.

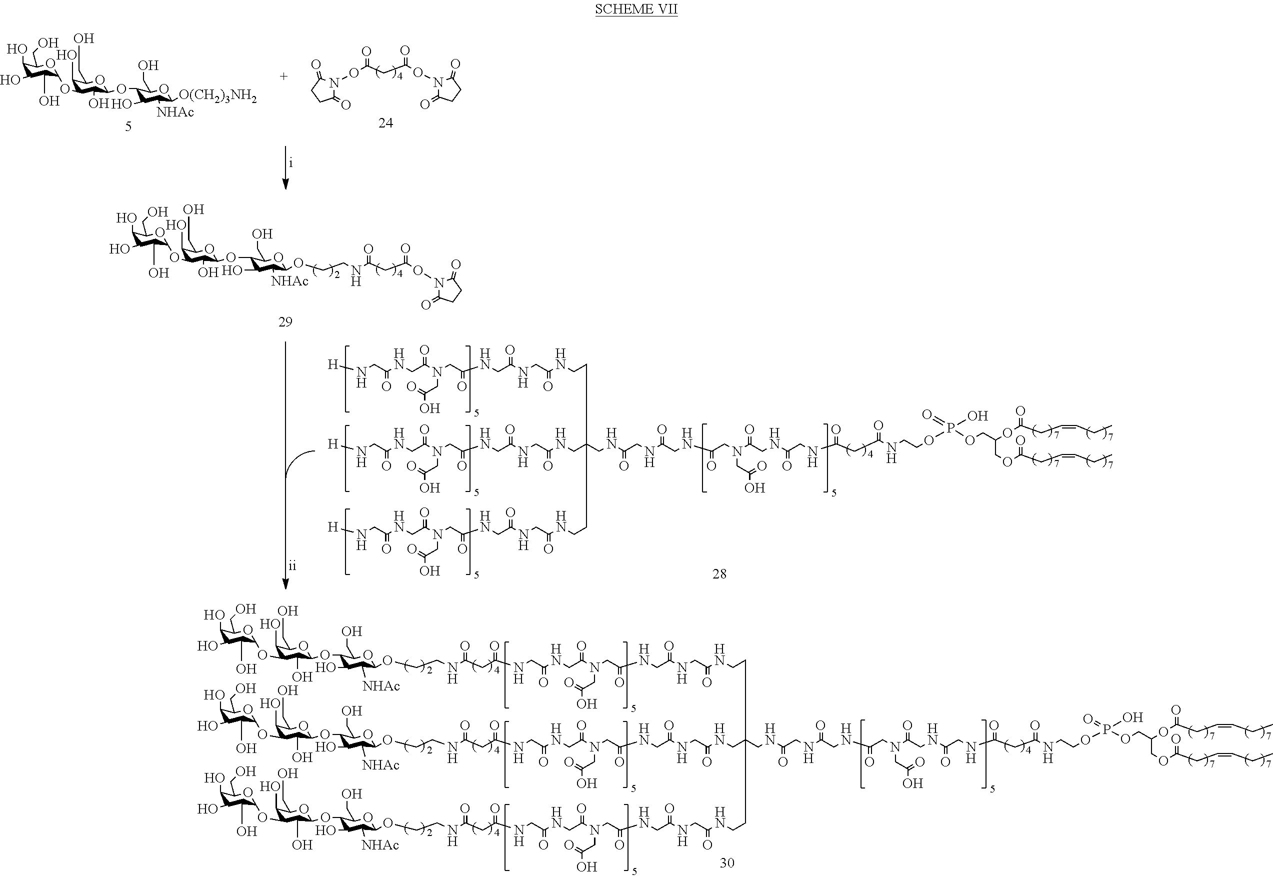

### Preparation of Galili-T-17-DE (30)(Scheme VII)

Compound 28 (4.3 mg, 5 μmol) and Et₃N (0.5 μl) in H₂O (0.75 ml) was added to a stirred solution of compound 29 (5 mg, 6 μmol) in dry DMSO (0.3 mL) in 3 portions during 1.5 h. The mixture was stirred for 24 h at room temperature and then subjected to column chromatography (Sephadex LH-20, MeOH—H₂O, 3:7) to yield the crude product 30. The product was lyophilized from water, the residue was dissolved in 3 ml of water, aqueous solution of NaHCO₃ (10 mM) was added to pH 6.5 and the solution was lyophilized to provide 3.7 mg of the compound 30 as Na-salt.

¹H NMR (700 MHz, D₂O/CD₃OD, 2:1 (v/v), selected chemical shifts) δ, ppm: 1.06 (t, J 7.03 Hz, CH₃ of DE), 1.28-1.61 (m, CH₂ of DE), 1.71-1.88 (m, —COCH₂CH₂CH₂CH₂CO and —COCH₂CH₂—), 1.90-1.99 (m, OCH₂CH₂CH₂N), 2.13-2.27 (m, —CH₂CH═CHCH₂—, NHC(O)CH₃), 2.35-2.58 (m, COCH₂CH₂CH₂CH₂CO— and —COCH₂CH₂—), 2.93-3.24 (broad. s, 8H; 4C—CH₂NH), 4.63 (dd, J 2.49, J 12.32, C(O)OCHHCHOCH₂O—), 4.67 and 4.70 (2d, J_{1,2} 7.81, J_{1,2} 7.95, H-1^{I}, H-1^{II}), 5.30 (d, J_{1,2} 3.92, H-1^{III}), 5.42-5.47 (m, —OCH₂—CHO—CH₂O—), 5.52-5.58 (m, 4H, 2×-CH═CH—). MALDI TOF mass-spectrum, M/Z: 8188 (M+Na); 8204 (M+K); 8226 (MNa+K).

### Example 3: Preparation of the Compound of Formula (III) “GalNAc-Gal-GlcNAc-Ad-DOPE”

### Preparation of 3-aminopropyl 2-acetamido-2-deoxy-α-D-galactopyranosyl-(1→3)-β-D-galactopyranosyl-(1→4)-2-acetamido-2-deoxy-β-D-glucopyranoside (5) (Scheme I)

The glycosyl chloride 3,4,6-tri-O-acetyl-2-azido-2-desoxy-β-D-galactopyranosylchloride (1) was prepared according to the method disclosed in the publication of Paulsen et al (1978) Darstellung selektiv blockierter 2-azido-2-desoxy-_{d}-gluco-und-_{d}-galactophyranosylhalogenide: Reaktivität und ¹³C-NMR-Spektren Carbohydrate Research, 64, 339-364. The glycosyl acceptor (3-trifluoroacetamidopropyl)-2-acetamido-3-O-acetyl-6-O-benzyl-2-deoxy-4-O-(2,4-di-O-acetyl-6-O-benzyl-β-D-galactopyranosyl)-β-D-glucopyranoside (2) was prepared according to the method disclosed in the publication of Pazynina et al (2008) Russian Journal of Bioorganic Chemistry 34(5), 625-631.

A solution of the glycosyl acceptor (420 mg, 0.5 mmol), silver triflate (257 mg, 1.0 mmol), tetramethylurea (120 μl, 1.0 mmol) and freshly calcinated molecular sieves 4 Å in dry dichloromethane (20 ml), were stirred at room temperature in darkness for 30 min. Another portion of sieves 4 Å was added, and a solution of glycosyl chloride (350 mg, 1.0 mmol) in dry dichloromethane (3 ml) was added. The mixture was stirred for 20 h at room temperature. The resin was filtered and washed with methanol (4×10 ml), then solvent was evaporated. Chromatography on silica gel (elution with 5-7% isopropanol in chloroform) yielded 407 mg (70%) of the product 3 as a mixture of anomers (α/β=3.0 as determined by ¹H-NMR spectroscopy).

A solution of the product 3 (407 mg, 0.352 mmol) in methanol (30 ml) was subjected to hydrogenolysis over 400 mg 10% Pd/C for 16 h. Then the resin was filtered off, washed with methanol (4×10 ml) and the product concentrated in vacuum. The dry residue was acetylated with 2:1 pyridine-acetic anhydride mixture (6 ml) at 20° C. for 16 h, the reagents being co-evaporated with toluene. Two chromatography steps on silica gel (elution with 10% isopropanol in ethyl acetate and with 5-10% methanol in chloroform) resulted in 160 mg (42%) of the product 4 and 39 mg (10%) of the product 413.

A solution of 2 M sodium methylate in methanol (200 μl) was added to a solution of the product 4 (160 mg, 0.149 mmol) in dry methanol (4 ml). The solution was evaporated after 1 h, 4 ml water added and the solution kept for 16 h before being chromatographed on a Dowex-H⁺ column (elution with 1 M ammonia). The eluate was evaporated, lyophilized to yield 87.2 mg (91%) of the 3-aminopropyltrisaccharide (5).

¹H NMR spectra were recorded on a Bruker BioSpin GmbH spectrometer at 303K. Chemical shifts (δ) for characteristic protons are provided in ppm with the use of HOD (4.750), CHCl₃ (δ 7.270) as reference. Coupling constants (J) are provide in Hz. The signals in ¹H NMR spectra were assigned using a technique of spin-spin decoupling (double resonance) and 2D-¹H, ¹H-COSY experiments.

The values of optical rotation were measured on a digital polarimeter Perkin Elmer 341 at 25° C. Mass spectra were registered on a MALDI-TOF Vision-2000 spectrometer using dihydroxybenzoic acid as a matrix.

4: ¹H-NMR (700 MHz, CDCl₃): 1.759-1.834 (m, 1H, CH sp); 1.853-1.927 (m, 1H, CH sp); 1.972, 1.986, 1.996, 2.046, 2.053, 2.087, 2.106, 2.115, 2.130, 2.224 (10s, 10×3H, COCH₃); 3.222-3.276 (m, 1H, NCH sp); 3.544-3.583 (m, 1H, OCH sp); 3.591-3.661 (m, 2H, NCH sp, H-5a); 3.764 (dd≈t, 1H, H-4a, J 8.8); 3.787 (dd, 1H, H-3b, J_{3,4} 3.7, J_{2,3} 9.9); 3.836 (br. t, 1H, H-5b, J 7.3); 3.882-3.920 (m, 1H, OCH sp); 3.950 (dd, 1H, H-6′c, J_{6′, 6″} 10.6, J_{5,6′} 5.2); 4.009 (ddd, 1H, H-2a, J_{1,2} 7.9, J_{2,3} 10.0, J_{2,NH} 9.0); 4.076-4.188 (m, 5H, H-6′a, H-6′b, H-6″b, H-5c, H-6″c); 4.415 (d, 1H, H-1a, J_{1,2} 7.9); 4.443 (d, 1H, H-1b, J_{1,2} 7.9); 4.529 (dd, 1H, H-6″a, J_{6′, 6″} 12.0, J_{5,6′} 2.5); 4.548 (ddd, 1H, H-2c, J_{1,2} 3.4, J_{2,3} 11.6, J_{2,NH} 9.4); 4.893 (dd, 1H, H-3c, J_{3,4} 3.1, J_{2,3} 11.6); 5.021 (d, 1H, H-1c, J_{1,2} 3.4); 5.039-5.075 (m, 2H, H-3a, H-2b); 5.339 (dd≈d, 1H, H-4b, J 2.9); 5.359 (dd, 1H, H-4c, J_{3,4} 2.7, J_{4,5} 0.9); 5.810 (d, 1H, NHAc a, J_{2,NH} 9.0); 6.184 (d, 1H, NHAc c, J_{2,NH} 9.4); 7.310-7.413 (m, 1H, NHCOCF₃ sp). R_{f} 0.31 (EtOAc-iPrOH, 10:1). MS, m/z calculated for [C₄₃H₆₀N₃F₃O₂₅]H⁺: 1076.35, found 1076.

4β: ¹H-NMR (700 MHz, CDCl₃): 1.766-1.832 (m, 1H, CH sp); 1.850-1.908 (m, 1H, CH sp); 1.923, 1.969, 1.982, 2.059, 2.071, 2.099 (2), 2.120, 2.136, 2.148 (10s, 10×3H, COCH₃); 3.230-3.289 (m, 1H, NCH sp); 3.521 (ddd, 1H, H-2c, J_{1,2} 8.2, J_{2,3} 11.2, J_{2,NH} 7.8); 3.548-3.591 (m, 1H, OCH sp); 3.591-3.648 (m, 2H, NCH sp, H-5a); 3.743 (dd≈t, 1H, H-4a, J 8.6); 3.795 (br. t, 1H, H-5b, J 6.5); 3.852 (dd, 1H, H-3b, J_{3,4} 3.6, J_{2,3} 9.9); 3.873-3.923 (m, 2H, H-5c, OCH sp); 4.002 (ddd, 1H, H-2a, J_{1,2} 8.0, J_{2,3} 9.5, J_{2,NH} 8.9); 4.039 (dd, 1H, H-6′b, J_{6′, 6″} 11.6, J_{5,6′} 6.9); 4.087-4.144 (m, 3H, H-6′a, H-6″b, H-6′c); 4.160 (dd, 1H, H-6″c, J_{6′, 6″} 11.2, J_{5,6″} 6.0); 4.409, 4.417 (2d≈t, 2×1H, H-1a, H-1 b, J 7.6); 4.519 (dd, 1H, H-6″a, J_{6′,6″} 11.8, J_{5,6′} 2.5); 4.992 (d, 1H, H-1c, J_{1,2} 8.2); 5.043 (dd, 1H, H-3a, J_{3,4} 8.6, J_{2,3} 9.5); 5.066 (dd, 1H, H-2b, J_{1,2} 8.0, J_{2,3} 9.8); 5.350 (dd≈d, 1H, H-4c, J 3.2); 5.372 (dd≈d, 1H, H-4b, J 3.4); 5.399 (d, 1H, NHAc c, J_{2,NH} 7.8); 5.449 (dd, 1H, H-3c, J_{3,4} 3.4, J_{2,3} 11.3); 5.856 (d, 1H, NHAc a, J_{2,NH} 8.9); 7.361-7.466 (m, 1H, NHCOCF₃ sp). R_{f} 0.24 (EtOAc-iPrOH, 10:1). MS, m/z calculated for [C₄₃H₆₀N₃F₃O₂₅]H⁺: 1076.35, found 1076.

5: ¹H-NMR (700 MHz, D₂O): 1.924-2.002 (m, 2H, CH₂ sp); 2.060, 2.064 (2s, 2×3H, NCOCH₃); 3.102 (m≈2H, NCH₂ sp, J 6.8); 3.592-3.644 (m, 1H, H-5a); 3.655 (dd, 1H, H-2b, J_{1,2} 7.9, J_{2,3} 9.9); 3.702 (br. dd, 1H, H-5b, J_{5,6′} 3.8, J_{5,6″} 8.2, J_{4,5}≤1); 3.713-3.815 (m, 9H); 3.846 (dd, 1H, H-6′a, J_{6′,6″} 12.3, J_{5,6}′ 5.3); 3.984-4.062 (m, 4H, OCH sp, H-6″a, H-4b, H-3c); 4.123 (dd≈d, 1H, H-4c, J 2.9); 4.206 (br. t, 1H, H-5c, J 6.3); 4.248 (dd, 1H, H-2c, J_{1,2} 3.6, J_{2,3} 11.0); 4.542 (2d≈t, 2H, H-1a, H-1b, J 7.4); 5.100 (d, 1H, H-1c, J_{1,2} 3.5). R_{f} 0.55 (MeOH-1M aq. Py.AcOH, 5:1). MS, m/z calculated for [C₂₅H₄₅N₃O₁₆]H⁺: 644.28; found 644. [α]_{546 nm}+128 (c 0.3; MeCN-H₂O, 1:1).

5β: ¹H-NMR (700 MHz, D₂O): 1.938-1.991 (m, 2H, CH₂ sp); 2.055, 2.062 (2s, 2×3H, NCOCH₃); 3.100 (m≈t, 2H, NCH₂ sp, J 6.9); 3.610 (dd, 1H, H-2b, J_{1,2} 7.9, J_{2,3} 9.9); 3.603-3.636 (m, 1H, H-5a); 3.682 (br. dd, 1H, H-5b, J_{5,6′} 4.9, J_{5,6′} 7.8, J_{4,5}≤1); 3.693-3.826 (m, 11H); 3.842 (dd, 1H, H-6′a, J_{6′, 6″} 12.1, J_{5,6′} 5.2); 3.934-3.972 (m, 2H, H-4b, H-2c); 4.012 (dd, 1H, H-6″a, J_{6′, 6″} 12.2, J_{5,6″} 2.0); 4.023-4.057 (m, 1H, OCH sp); 4.175 (dd≈d, 1H, H-4c, J 2.9); 4.478 (d, 1H, H-1 b, J_{1,2} 7.9); 4.531 (d, 1H, H-1a, J_{1,2} 8.1); 4.638 (d, 1H, H-1c, J_{1,2} 8.4). R_{f} 0.48 (MeOH-1M aq. Py.AcOH, 5:1). MS, m/z calculated for [C₂₅H₄₅N₃O₁₆]H⁺: 644.28; found 644. [α]_{546 nm}+6 (c 0.3; MeCN-H₂O, 1:1).

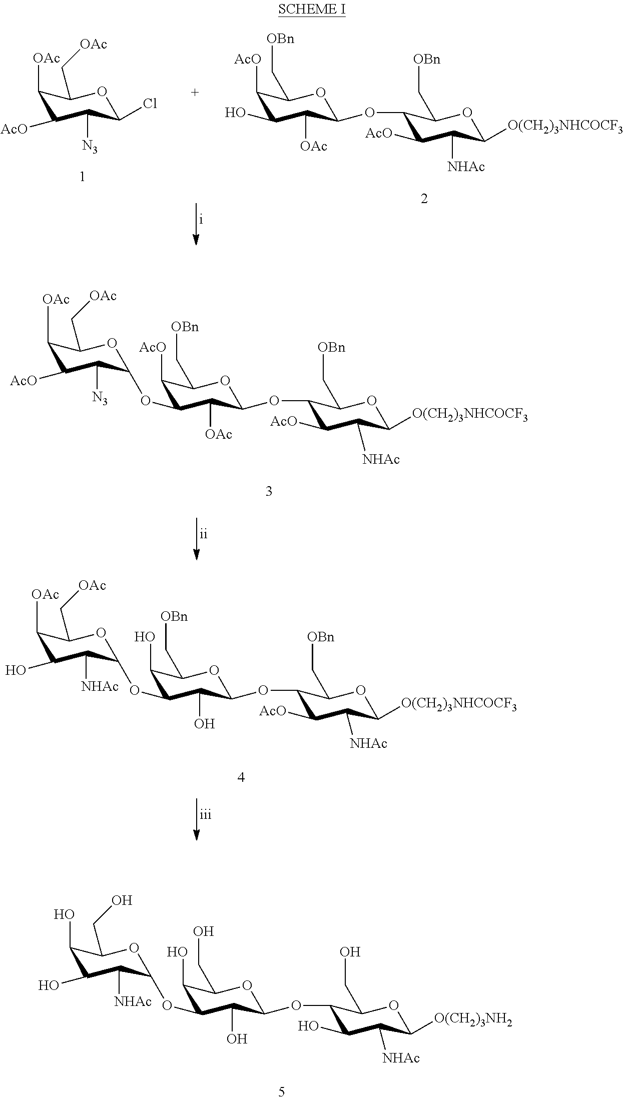

### Preparation of Activated 1,2-O-dioleoyl-sn-glycero-3-phosphatidylethanolamine (DOPE-Ad-ONSu)(8) (Scheme II)

To a solution of bis(N-hydroxysuccinimidyl) adipate (6) (70 mg, 205 μmol) in dry N,N-dimethylformamide (1.5 ml), 1,2-O-dioleoyl-sn-glycero-3-phosphatidylethanolamine (7) (40 μmol) in chloroform (1.5 ml) was added, followed by triethylamine (7 μl). The mixture was kept for 2 h at room temperature, then neutralized with acetic acid and partially concentrated under vacuum. Column chromatography (Sephadex LH-20, 1:1 chloroform-methanol, 0.2% acetic acid) of the residue yielded the product 8 (37 mg, 95%) as a colorless syrup.

¹H NMR spectra were acquired on a Bruker DRX-500 spectrometer. Chemical shifts are provided in ppm (δ) relative to CD₃OD. TLC was performed on silica gel 60 F₂₅₄ plates (Merck) with compounds detected by staining with 8% of phosphoric acid in water followed by heating at over 200° C.

8: ¹H NMR (CDCl₃/CD₃OD, 2:1) 5.5 (m, 4H, 2×(—CH═CH—), 5.39 (m, 1H, —OCH₂—C₁₂O—CH₂O—), 4.58 (dd, 1H, J=3.67, J=11.98, —CCOOHCH—CHO—CH₂O—), 4.34 (dd, 1H, J=6.61, J=11.98, —CCOOHCH—CHO—CH₂O—), 4.26 (m, 2H, PO—CH₂—CH₂—NH₂), 4.18 (m, 2H, —CH₂—OP), 3.62 (m, 2H, PO—CH₂—CH₂—NH₂), 3.00 (s, 4H, ONSuc), 2.8 (m, 2H, —CH₂—CO (Ad), 2.50 (m, 4H, 2×(—CH₂—CO), 2.42 (m, 2H, —CH₂—CO (Ad), 2.17 (m, 8H, 2×(—CH₂—CH═CH—CH₂—), 1.93 (m, 4H, COCH₂CH₂CH₂CH₂CO), 1.78 (m, 4H, 2×(COCH₂CH₂—), 1.43, 1.47 (2 bs, 40H, 20 CH₂), 1.04 (m, 6H, 2 CH₃). R_{f} 0.5 (chloroform-methanol-water, 6:3:0.5.

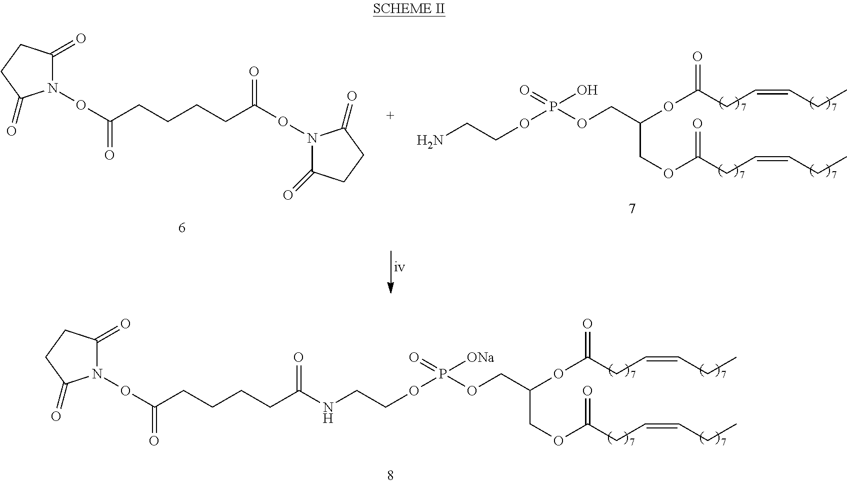

### Preparation of GalNAcα1-3Galβ1-4GlcNAc-Ad-DOPE (9) (Scheme III)

To a solution of the product 8 (33 μmol) in N,N-dimethylformamide (1 ml), 30 μmol of the 3-aminopropyltrisaccharide 5 and 5 μl of triethylamine (Et₃N) were added. The mixture was stirred for 2 h at room temperature. Column chromatography on silica gel (CH₂Cl₂-EtOH-H₂O; 6:5:1) provided an 81% yield of the construct 9.

9: ¹H NMR (700 MHz, CDCl₃-CD₃OD, 1:1 v/v, selected), δ, ppm: 1.05 (t, 6H, J 7.05, 2CH₃), 1.39-1.55 (m, 40H, 20CH₂), 1.75-1.84 (m, 8H, COCH₂CH₂CH₂CH₂CO and 2×COCH₂CH₂—), 1.84-1.96 (m, 2H, O—CH₂CH₂CH₂—NH), 2.15-2.22 (m, 14H, 2×(—CH₂—CH═CH—CH₂—), 2×NHC(O)CH₃), 2.34-2.46 (m, 4H, 2×-CH₂—CO), 2.36-2.44 (m, 4H, 2×-CH₂—CO), 3.29-3.34 (m, 1H, —CH₂—CHH—NH), 4.17-4.20 (m, 2H, —CHO—CH₂OP—), 4.34-4.39 (m, 2H, —CH₂OPO—CH₂—CH₂), 4.57 (d, 1H, J_{1,2} 8.39, H-1^{I}), 4.50 (dd, 1H, J 3.78, J 10.82, —C(O)OCHHCHOCH₂O—), 4.58-4.61 (m, 2H, H-1″, C(O)OCHHCHOCH₂O—), 5.15 (d, 1H, J_{1,2} 3.76, H-1^{III}), 5.38-5.42 (m, 1H, —OCH₂—CHO—CH₂O—), 5.47-5.53 (m, 4H, 2×-CH═CH—). R_{f} 0.5 (CH₂Cl₂EtOH-H₂O; 6:5:1).

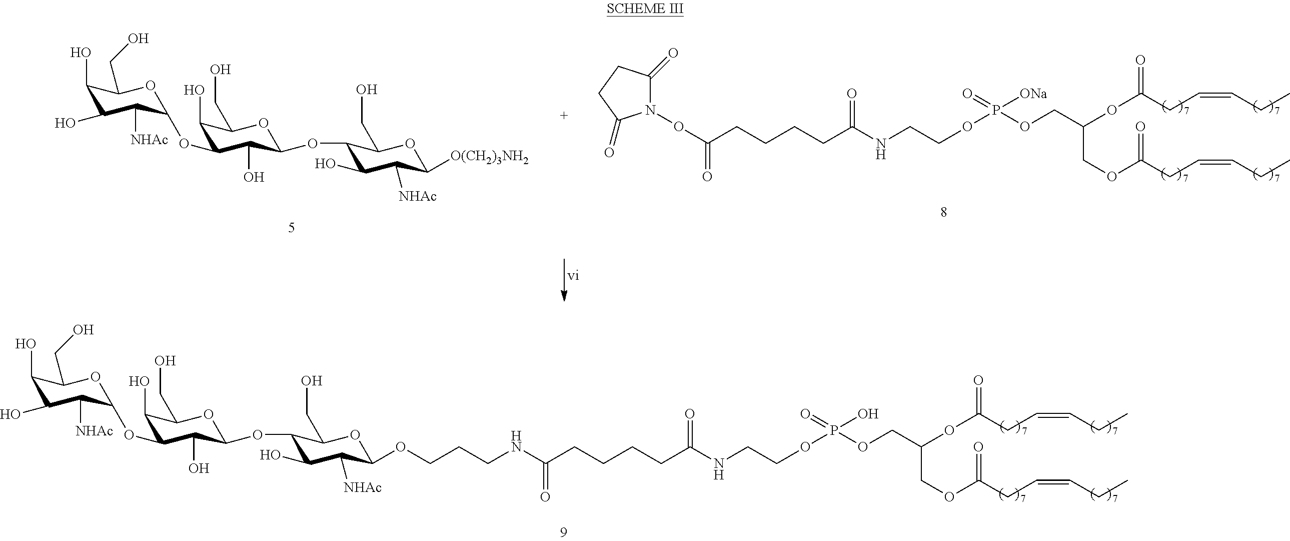

### Biological Data

### Anti-Gal Recruitment Assay

CHO-K1 cells were harvested from cell culture flasks, counted and resuspended in PBS to a cell density of 5×10⁶ cells/ml. Each glycolipid was serially diluted in PBS across nine 1.5 ml centrifuge tubes so that the final volume in the tubes was 100 μl. To each tube, 100 μl of the CHO-K1 cell suspension was added and the tubes incubated for 1 hour at 37° C. After an hour the cells were pelleted by centrifugation at 400 g for 3 minutes and resuspended in 500 μl of PBS+0.1% BSA. This was repeated twice more to wash the cells. After the final wash the cells were resuspended in 100 μl of monoclonal anti-Gal IgG1 diluted 1:8 in PBS+0.1% BSA. The tubes were incubated on ice for 30 minutes. After 30 minutes the cells were pelleted by centrifugation at 400 g for 3 minutes and resuspended in 500 μl of PBS+0.1% BSA. This was repeated twice more to wash the cells. After the final wash the cells were resuspended in 100 μl of FITC-conjugated mouse anti-human IgG (Biolegend) and the tubes incubated on ice for 30 minutes. After 30 minutes the cells were pelleted by centrifugation at 400 g for 3 minutes and resuspended in 500 μl of PBS+0.1% BSA. This was repeated twice more to wash the cells. After the final wash the cells were resuspended in 200 μl of PBS+0.1% BSA containing 2.5 μl of 7-AAD (Biolegend). After 5 minutes incubation on ice the cells were analysed on a Cytomics FC500 flow cytometer (Beckman Coulter). Dead cells were excluded from the analysis.

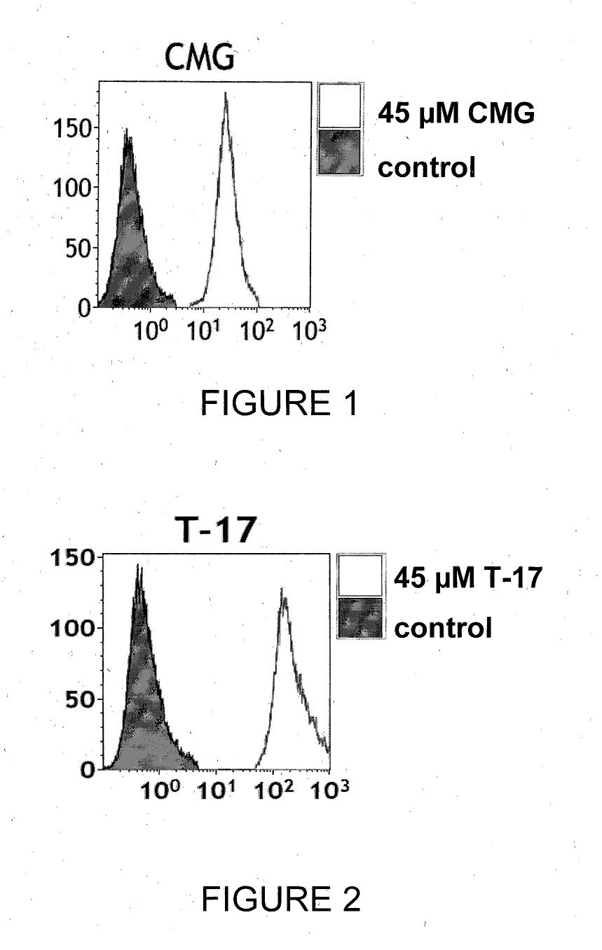

The compounds as prepared herein as Example 1 (Galili-CMG2-DOPE) and Example 2 (Galili-T17 DOPE) were tested in the anti-gal recruitment assay and the results may be seen in FIGS. 1 and 2. These results demonstrate that the compound as prepared herein as Example 1 (Galili-CMG2-DOPE) which is an alpha-Gal glycolipid having a CMG spacer between a single alpha-Gal sugar and a single lipid portion of the molecule incorporates into the plasma membrane of CHO-K1 cells and presents the alpha-Gal epitope for recognition by anti-Gal antibodies (see FIG. 1). The results also demonstrate that the compound as prepared herein as Example 2 (Galili-T17 DOPE) which is a mixture of glycolipids having a single lipid portion attached to two or three alpha-Gal sugars by branched CMG linkers incorporates into the plasma membrane of CHO-K1 cells and recruits more anti-Gal antibody than an equivalent concentration of the single alpha-Gal molecule of Example 1.

### Complement Dependent Cytotoxicity Assay

CHO-K1 cells were harvested from cell culture flasks, counted and resuspended in PBS to a cell density of 5×10⁶ cells/ml. Each glycolipid was serially diluted in PBS across nine 1.5 ml centrifuge tubes so that the final volume in the tubes was 100 μl. To each tube, 100 μl of the CHO-K1 cell suspension was added and the tubes incubated for 1 hour at 37° C. After an hour the tubes were placed on ice for 5 minutes and then the cells washed 3 times with 500 μl of ice-cold PBS. The cells were resuspended in a final volume of 250 μl of ice-cold PBS and 50 μl aliquots were transferred to duplicate wells of a 96 well plate. To each well containing cells 50 μl of 100% normal or heat-inactivated (30 minutes at 56° C.) human serum complement (Innovative Research) was added so that the final concentration of human serum was 50%. The plate was incubated at 37° C. for 1 hour, after which cell viability was measured using CellTiter-Glo reagent (Promega) read on a EnVision plate reader (Perkin Elmer).

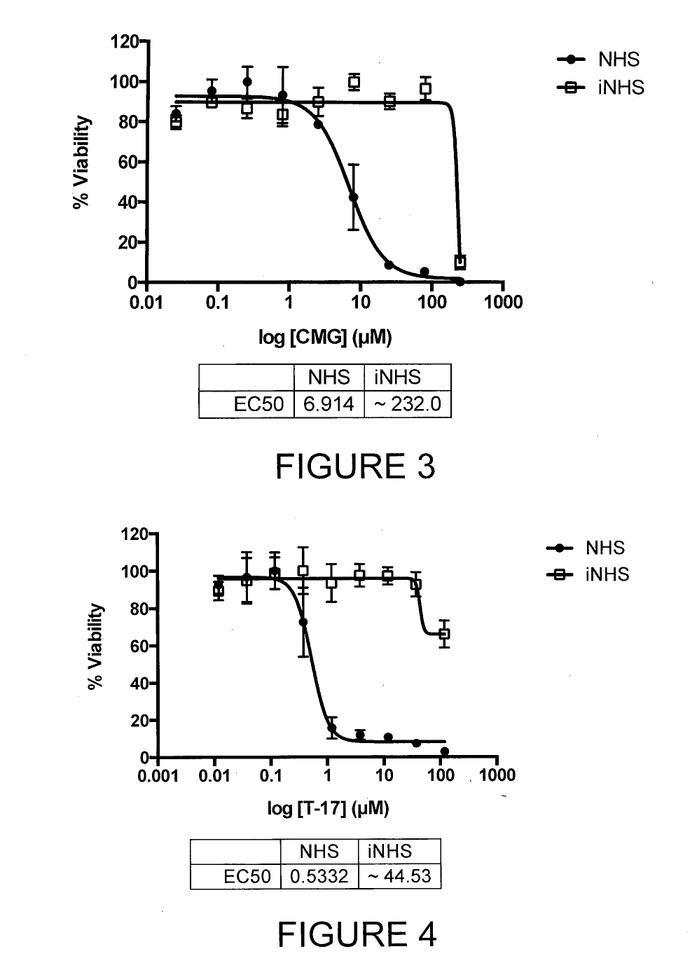

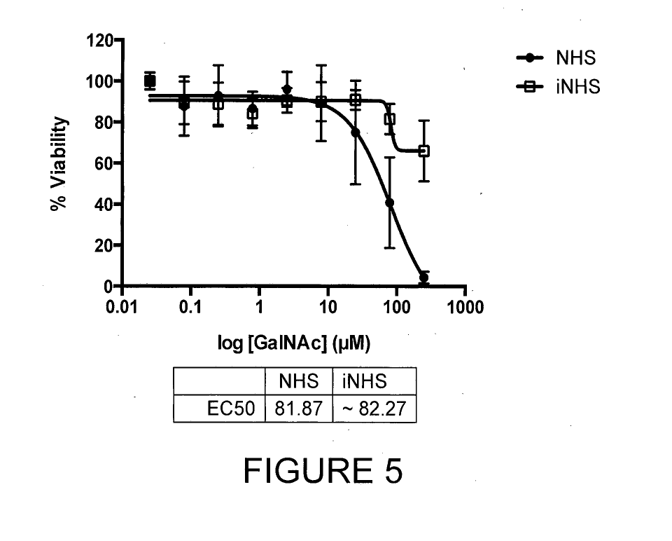

The compounds as prepared herein as Example 1 (Galili-CMG2-DOPE), Example 2 (Galili-T17 DOPE) and Example 3 (GalNAc-Gal-GlcNAc-Ad-DOPE) were tested in the complement dependent cytotoxicity assay and the results may be seen in Table 1 below and FIGS. 3 to 5.

TABLE 1
Results of Complement Dependent Cytotoxicity Assay

| | EC50 | 95% Confidence |
|---|---|---|
| Compound | (μM) | Interval |
| Example 1 | 7.02 | 3.2-10.9 |
| (Galili-CMG2-DOPE) | | |
| Example 2 | 0.539 | 0.4-0.7 |
| (Galili-T17 DOPE) | | |
| Example 3 | 86.8 | −14.6-188.2 |
| (GalNAc-Gal-GlcNAc-Ad-DOPE) | | |

These results demonstrate that CHO-K1 cells labelled with the compound as prepared herein as Example 1 (Galili-CMG2-DOPE; i.e. the single alpha-Gal CMG molecule) are lysed by human serum complement (see FIG. 3). The results also demonstrate that CHO-K1 cells labelled with the compound as prepared herein as Example 2 (Galili-T17 DOPE; i.e. the dimeric/trimeric alpha-Gal molecule) are more susceptible to lysis by human serum complement than cells incubated with the same concentration of single alpha-Gal molecule (i.e. the compound as prepared herein as Example 1 (Galili-CMG2-DOPE). The results also demonstrate that CHO-K1 cells labelled with the compound as prepared herein as Example 3 (GalNAc-Gal-GlcNAc-Ad-DOPE; i.e. the glycolipid molecule that has a GalNAc alpha sugar antigen) are lysed by human serum complement.

1. A glycolipid compound selected from a compound of formula (I), (II) and (III) or a pharmaceutically acceptable salt thereof:

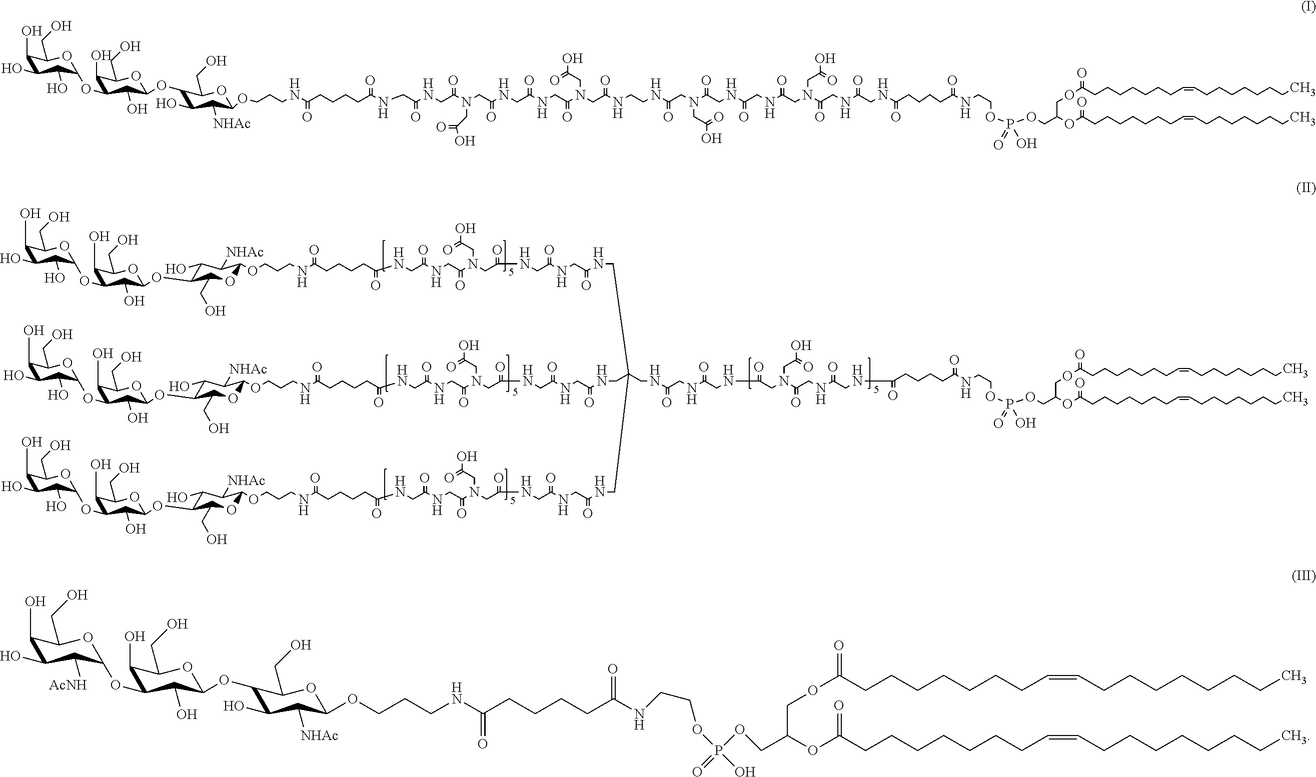

2. A pharmaceutical composition comprising the glycolipid compound or a pharmaceutically acceptable salt thereof as defined in claim 1.

3. A method for treating a tumour in a subject comprising administering the pharmaceutical composition as defined in claim 2.

4. The method as defined in claim 3, wherein the tumour is a solid tumour, myeloma, or a lymphoma.

5. The method as defined in claim 3, wherein the tumour is a tumour originating from an organ selected from peritoneum, liver, pancreas, lung, urinary bladder, prostate, uterus, cervix, vagina, bone marrow, breast, skin, brain, lymph node, head and neck, stomach, intestine, colon, kidney, testis, and ovaries.

6. The method as defined in claim 3, wherein the tumour comprises a primary tumour and/or a metastasis.

7. The method as defined in claim 3, wherein the tumour comprises melanoma, sarcoma, glioma, or carcinoma cells.

8. The method as defined in claim 3, wherein the administering is by injection.

9. The method as defined in claim 3, wherein the administering is by one dose or multiple doses.

10. The method as defined in claim 3, wherein the administering step comprises administration by topical application.

11. The method as defined in claim 3, wherein the pharmaceutical composition additionally comprises one or more pharmaceutically acceptable carrier(s), diluents(s) and/or excipient(s).

12. The method as defined in claim 3, wherein the pharmaceutical composition additionally comprises one or more additional therapeutic agents.

13. The method as defined in claim 12, wherein the one or more additional therapeutic agents comprise one or more systemic inhibitors of immune system down-regulation, such as anti-CTLA-4, anti-PD-1 and anti-PD-L1 antibodies, in particular anti-PD-1 antibodies.

14. A method of treating a tumour in a subject, comprising:
a) providing:
i) a subject comprising at least one tumour that comprises a plurality of cancer cells having a cell surface; and
ii) the pharmaceutical composition as defined in claim 2; and
b) introducing said glycolipid or composition into the tumour.

15. The method as defined in claim 14, wherein the subject is a human or a mouse.

16. The method as defined in claim 14, wherein the introducing step comprises a procedure selected from: injection, imaging guided injection, endoscopy, bronchoscopy, cystoscopy, colonoscopy, laparoscopy, and catheterization.

17. The method as defined in claim 14, which additionally comprises inducing an intratumoural inflammation.

18. The method as defined in claim 14, wherein the subject was treated previously to surgically remove the tumour.

19. The method as defined in claim 14, wherein the subject was not treated previously to remove the tumour.

20. The method as defined in claim 14, wherein the tumour undergoes regression or is destroyed.

21. The method as defined in claim 14, wherein the introducing step further comprises regression or destruction of a second tumour in the subject.

22. The method as defined in claim 10, wherein the composition is a topical ointment, topical lotion or topical solution.
